(19) United States
(12) Patent Application Publication
(10) Pub. No.: US 2017/0106017 A1
(43) Pub. Date: Apr. 20, 2017

(54) METHODS AND COMPOSITIONS FOR TREATMENT OF MACROPHAGE-RELATED DISORDERS

(71) Applicants: Neuraltus Pharmaceuticals, Inc., San Bruno, CA (US); The Regents of the University of California, Oakland, CA (US)

(72) Inventors: Michael S. McGRATH, Burlingame, CA (US); Gilbert BLOCK, San Bruno, CA (US)

(21) Appl. No.: 15/311,036

(22) PCT Filed: May 15, 2015
(86) PCT No.: PCT/US2015/031145

§ 371 Date: Nov. 14, 2016

Related U.S. Application Data
(60) Provisional application No. 61/994,736, filed on May 16, 2014.
(60) Provisional application No. 62/051,849, filed on Sep. 17, 2014.

(51) Int. Cl. A61K 33/20; G01N 33/68

(52) U.S. Cl. CPC A61K 33/20; G01N 33/6896; G01N 2800/52; G01N 2800/2835; G01N 2333/70535; G01N 2333/70539

(57) ABSTRACT

Provided herein are methods and composition for the treatment for macrophage-related disorders, for example through the use of biomarkers for selection of responders and treatment monitoring.

This application claims the benefit to U.S. Provisional Application No. 61/994,736, filed on May 16, 2014, and U.S. Provisional Application No. 62/051,849, filed on Sep. 17, 2014, each of which is incorporated herein by reference in its entirety.

### BACKGROUND OF THE INVENTION

Macrophages are white blood cells produced by the division of monocytes. Monocytes and macrophages are phagocytes, and play a role in innate immunity (non-specific immune defenses) as well as helping to initiate adaptive immunity (specific defense mechanisms). These cells phagocytose (engulf and then digest) cellular debris and pathogens either as stationary or as mobile cells. When activated by pathogens or by other mechanisms, macrophages stimulate and recruit lymphocytes and other immune cells to respond to the insult. Activated macrophages are involved in the progression of a number of diseases and disorders. Activated macrophages elicit massive leukocyte infiltration and flood the surrounding tissue with inflammatory mediators, pro-apoptotic factors, and matrix degrading proteases. These actions can result in inflammation that can dismantle tissues to the point of inflicting serious injury. Tissue destruction perpetrated by macrophage-induced inflammation has been associated with the development of degenerative diseases, tumors, autoimmune disorders, and other conditions.

Oxidative agents such as chlorite can return macrophages to their inactivated state. Chlorite has been used to treat various diseases or conditions. For example, chlorite has been used to treat macrophage-related diseases such as amyotrophic lateral sclerosis (ALS) and Alzheimer's disease (AD). However, the effectiveness of the chlorite treatment on all patients suffering from the diseases can vary. The present invention provides methods for treating sub-populations of patients suffering from macrophage-related diseases and related conditions with chlorite, as well as monitoring the treatment with chlorite.

### SUMMARY OF THE INVENTION

The present invention provides a method of treating a subject suffering from a macrophage-related disease. The method can comprise steps of: (a) selecting a subject suffering from a macrophage-related disease if said subject has an elevated plasma level of one or more inflammatory factors chosen from the group consisting of LPS, IL-6, IL-8, IL-18, IFN-g, and CRP; and (b) administering to the subject a therapeutically effective amount of a pharmaceutical composition comprising chlorite.

The present invention provides a method of treating a subject suffering from a macrophage-related disease. The method can comprise steps of: (a) selecting a subject suffering from a macrophage-related disease if said subject has an elevated plasma level of one or more inflammatory factors chosen from the group consisting of LPS, IL-6, IL-8, IL-18, IFN-g, and CRP; and (b) administering to the subject a therapeutically effective amount of a pharmaceutical composition comprising chlorite.

In one aspect, the one or more inflammatory factors is IL-18. The plasma level of IL-18 prior to said administering can be at least about 60 pg/ml. The plasma level of IL-18 in said subject can decrease after said administering.

In another aspect, the subject can further have an elevated plasma level of one or more inflammatory factors selected form the group consisting of: LPS, IL-6, IL-8, IFN-g, and CRP. In some cases, the one or more inflammatory factors is LPS. In another case, the subject can further have an elevated plasma level of one or more inflammatory factors selected from the group consisting of IL-18, IL-6, IL-8, IFN-g, and CRP.

In some cases, the plasma level of LPS prior to said administering is at least about 0.05, 0.1, 0.15, or 0.2 EU/ml. In some cases, the plasma level of LPS prior to said administering is at least about 0.05 EU/ml. In still yet another case, the plasma level of LPS can be higher than the normal level. The plasma level of LPS in said subject can decrease after said administering. In some cases, the plasma level of LPS in said subject can decrease to an undetectable level after said administering.

In some cases, the subject has elevated plasma levels of IL-6 and IFN-g. In practicing any of the methods as described herein, the plasma level of IL-6 can be at least about 6 pg/ml. The plasma level of IFN-g can be at least about 20 pg/ml. The plasma level of CRP can be at least about 1000 ng/ml. The subject can have an elevated plasma level of at least two inflammatory factors chosen from the group consisting of LPS, IL-6, IL-8, IL-18, IFN-g and CRP.

In another aspect, the macrophage-related disease can be selected from amyotrophic lateral sclerosis (ALS), Alzheimer's disease (AD), Parkinson's disease (PD) and HIV-associated neurocognitive disorder (HAND). The macrophage-related disease can be amyotrophic lateral sclerosis (ALS). In some cases, the subject was diagnosed as having the macrophage-related disease less than 3 years prior to said administering. In some cases, said subject does not show disease progression for at least 6 months after said administering.

In one aspect, said chlorite can be administered in an amount of at least about 1 mg or at least about 2 mg/kg body weight. Said composition can be administered intravenously. Said composition can be administered at least twice, three times or five times per month. Said composition can be administered for at least 2, 3, 4, 5 or 6 months.

In practicing any of the methods as described herein, the chlorite can be greater than 95%, 99% or 99.5% pure. The composition comprising chlorite can further comprise a pH adjusting agent. The composition can be a liquid that exhibits 25% less pH drift compared to an identical composition without said pH adjusting agent. The pH adjusting agent can be a phosphate buffer.

In some cases, said chlorite is sodium chlorite. In some cases, the chlorite is in a form of WF10.

Present invention also provides a method of monitoring the inflammation progress a macrophage-related disease in a subject. The method can comprise the steps of: (a) administering to the subject a pharmaceutical composition comprising chlorite; (b) measuring the plasma level of at least one monocyte activation marker selected from the group consisting of HLA-DR and CD 16; (c) comparing the measured plasma level of said monocyte activation marker to a plasma level of said monocyte activation marker in the subject prior to said administering step; and (d) continuing to administer the pharmaceutical composition to the patient if the plasma level of said monocyte activation marker has decreased as compared to the plasma level of said monocyte activation marker prior to said administering. In some cases, the plasma level of said monocyte activation marker is higher than normal level prior to said administering. In some cases, the plasma level of said monocyte activation marker decreases after said administering.

The plasma level of at least one monocyte activation marker can be measured 24 hours prior to said administering or 24 hours after said administering. The monocyte activation marker can be HLA-DR. In some cases, the subject has plasma level of HLA-DR higher than normal level prior to said administering. In some cases, said subject has decreased HLA-DR plasma level after said administering. Said method can further comprise measuring the plasma level of CD14. In some cases, the plasma level of CD14 in said subject can be higher than normal level prior to said administering. In some cases, the plasma level of CD14 decreases after said administering.

The monocyte activation marker can be CD16. In some cases, the plasma level of CD16 is higher than normal level prior to said administering. In some cases, the plasma level of CD16 decreases after said administering.

The plasma level of monocyte activation marker can be correlated with the rate of progression of said monocyte-related disease. In some cases, the elevated plasma level of HLA-DR and CD16 increase the rate of progression of said macrophage-related disease. Administering said composition can decrease the progression of said macrophage-related disease. In some cases, the administering said composition decreases the progression of said macrophage-related disease by at least 1.0 unit/month using the ALSFRS-R scoring scale. In some cases, the progression is decreased by at least 0.5 unit/month using the ALSFRS-R scoring scale. In some cases, the subject suffering from a macrophage-related disease has progression rate of at least 1.0 unit/month using the ALSFRS-R scoring scale.

In another aspect, the macrophage-related disease can be selected from amyotrophic lateral sclerosis (ALS), Alzheimer's disease (AD), Parkinson's disease (PD) and HIV-associated neurocognitive disorder (HAND). The macrophage-related disease can be amyotrophic lateral sclerosis (ALS).

### INCORPORATION BY REFERENCE

All publications, patents, and patent applications mentioned in this specification are herein incorporated by reference to the same extent as if each individual publication, patent or patent application was specifically and individually indicated to be incorporated by reference. In case of any inconsistency between the incorporated by reference publications and the instant specification, the instant specification will control.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows the overall design of the clinical trial.

FIG. 2 depicts a diagram of the clinical study flow and patient disposition to evaluate the effects of chlorite in treating ALS.

FIG. 3A shows the ALSFRS-R slope after six months of treatment without (left) and with (right) historical controls.

FIG. 3B shows the mean change from baseline in ALSFRS-R score at Week 25 without (left) and with (right) historical controls.

FIG. 3C shows the ALSFRS-R slope after six months of treatment in patients with baseline wrCRP greater than or equal to the baseline median wrCRP.

FIG. 4 shows the working mechanism of inflammation and ALS. LPS induces macrophage activation and production of NF-kB regulated factors. Plasma LPS would disappear after macrophage function turning back to normal.

FIG. 5 shows the working mechanism of chlorite in treating microphage-related diseases.

FIG. 6 shows the ALSFRS-R score over the course of 6 months of treatment in responders and non-responders. “Responders” are the sub-population of the subjects that respond positively to the sodium chlorite treatment. “Non-responders” are the sub-population of the subjects that do not respond positively in terms of the ALSFRS-R score to the sodium chlorite treatment.

FIG. 7 shows the percentage of patients who were stable or improved on change from baseline ALSFRS-R score after six months of treatment.

FIG. 8 is a chart showing the difference in the normalized baseline level of the inflammation plasma factors in the responders vs. non-responders. Responders have elevated plasma inflammation markers at baseline.

FIG. 9 is a chart showing the difference in the normalized baseline level of the inflammation plasma factors in the responder, placebo group and non-responders. Placebo group shows an intermediate level of inflammation consistent.

FIG. 10 is a ROC curve for comparing the area under the curve for each marker's ability to predict responders.

FIG. 11 is a table showing the baseline level of the inflammatory plasma factors in the responders vs. non-responders treated with 2 mg/kg of sodium chlorite.

FIG. 12 shows the plasma level some inflammatory factors at baseline and week 25 for responders, non-responders and placebo non-progressors. The “placebo non-progressor” refers to a sub-population of the placebo group that does not show disease progression in the duration of the study.

FIG. 13 shows the plasma level IL-18, CRP, IL-8, wrCRP, INF-g and IL-6 at baseline and Week 25 for responders and placebo non-progressors.

FIG. 14 shows mean plasma IL-18 levels in high dose “responders” vs. “non-responders” at baseline and following 6-month treatment period (Week 25). Error bars represent standard deviation.

FIG. 15 shows the IL-18 levels at baseline and Week 25 in responders, non-responders and placebos.

FIG. 16 is a box and whisker plot of the distribution of the log of IL-18, showing that the IL-18 levels at baseline can differentiate responders, and non-responders.

FIG. 17 is a table showing the baseline inflammation factor plasma baseline value interrelationships.

FIG. 18 shows mean plasma LPS in all patients treated with 1 mg/kg or 2 mg/kg chlorite/NP001 at baseline and following 6 month treatment period (Week 25). Error bars represent standard deviation. Limit of detection (LOD) for LPS=0.05.

FIG. 19 shows mean LPS in placebo “responders” and “non-responders” at baseline and following 6 month treatment period (Week 25). Error bars represent standard deviation. Limit of detection (LOD) for LPS=0.05.

FIG. 20 shows the plasma level IL-18 at baseline and Week 25 for each subject participating in the study.

FIG. 21 indicates a cut-off threshold value of the plasma level of IL-18 at baseline.

FIG. 22 shows LPS positive and negative patients at baseline and ALS disease progression rate.

FIG. 23 indicates ALS LPS negative patients have higher baseline ALSFRS-R scores.

FIG. 24 shows ALS LPS negative placebo patients become LPS positive within 6 months.

FIG. 25 shows decrease in ALSFRS-R score in ALS LPS negative patients within 6 months.

FIG. 26 shows the relationship between baseline monocyte inflammatory activation-related markers and the historic rate of ALS disease progression, assessed by average monthly change on ALSFRS-R the disease progression rate (ALSFRS-R Score loss per month) in ALS. FIG. 26A shows levels of baseline monocyte activation defined by CD14 co-expression of HLA-DR was directly related to the rate of ALS disease progression (r=0.4310, p=0.0138; n=32). FIG. 26B depicts positive correlation was observed between baseline levels of CD16 expression on the CD16 bright subset of monocytes and disease progression rate in ALS (r=0.4499, p=0.0098; n=32).

FIG. 27 shows NP001 treatment changes CD14 monocyte expression of HLA-DR as a function of the degree of monocyte HLA-DR expression at baseline.

FIG. 28 shows the comparison of NP001 treatment response between ALS patients with elevated levels of baseline monocyte HLA-DR and those with lower range of baseline monocyte HLA-DR.

FIG. 29 illustrates the greatest change in monocyte levels of HLA-DR in ALS patients with the highest rate of disease progression.

FIG. 30 shows NP001 induced changes from baseline on CD16 levels expressed on a CD16 bright subset of monocytes in a dose-dependent manner.

FIG. 31 shows the comparison of CD16 expression on monocyte CD16 bright subset in patients receiving 1.6 mg/kg dose NP001 relative to healthy controls.

### DETAILED DESCRIPTION OF THE INVENTION

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

“Treatment”, “treating”, “palliating” and “ameliorating”, as used herein, are used interchangeably. These terms refer to an approach for obtaining beneficial or desired results including but not limited to therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

As used herein, “agent” refers to a biological, pharmaceutical, or chemical compound or other moiety. Non-limiting examples include simple or complex organic or inorganic molecule, a peptide, a protein, an oligonucleotide, an antibody, an antibody derivative, antibody fragment, a vitamin derivative, a carbohydrate, a toxin, or a chemotherapeutic compound. Various compounds can be synthesized, for example, small molecules and oligomers (e.g., oligopeptides and oligonucleotides), and synthetic organic compounds based on various core structures. In addition, various natural sources can provide compounds for screening, such as plant or animal extracts, and the like. A skilled artisan can readily recognize that there is no limit as to the structural nature of the agents of the present invention.

Generally, the term “concurrent administration”, “co-administration”, or “administration in conjunction with” in reference to two or more subjects of administration for administration to a subject body, such as components, agents, substances, materials, compositions, and/or the like, refers to administration performed using dose(s) and time interval(s) such that the subjects of administration are present together within the subject body, or at a site of action in the subject body, over a time interval in less than de minimus quantities. The time interval may be any suitable time interval, such as an appropriate interval of minutes, hours, days, or weeks, for example. The subjects of administration may be administered together, such as parts of a single composition, for example, or otherwise. The subjects of administration may be administered substantially simultaneously (such as within less than or equal to about 5 minutes, about 3 minutes, or about 1 minute, of one another, for example) or within a short time of one another (such as within less than or equal to about 1 hour, 30 minutes, or 10 minutes, or within more than about 5 minutes up to about 1 hour, of one another, for example). The subjects of administration so administered may be considered to have been administered at substantially the same time. One of ordinary skill in the art will be able to determine appropriate dose(s) and time interval(s) for administration of subjects of administration to a subject body so that same will be present at more than de minimus levels within the subject body and/or at effective concentrations within the subject body. When the subjects of administration are concurrently administered to a subject body, any such subject of administration may be in an effective amount that is less than an effective amount that might be used were it administered alone.

The term “effective amount”, “therapeutic amount” or “therapeutic effective amount” which is further described herein, encompasses both this lesser effective amount and the usual effective amount, and indeed, any amount that is effective to elicit a particular condition, effect, and/or response. As such, a dose of any such subject of concurrent administration may be less than that which might be used were it administered alone. One or more effect (s) of any such subject (s) of administration may be additive or synergistic. Any such subject(s) of administration may be administered more than one time. The effective amount may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of proliferation or down-regulation of activity of a target protein. The specific dose will vary depending on the particular compounds chosen, the dosing regimen to be followed, whether it is administered in combination with other compounds, timing of administration, the tissue to which it is administered, and the physical delivery system in which it is carried.

A “therapeutic effect,” as used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The term “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

“Pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions of the invention is contemplated. Supplementary active ingredients can also be incorporated into the compositions.

“Subject” refers to an animal, such as a mammal, for example a human. The methods described herein can be useful in both human therapeutics, pre-clinical, and veterinary applications. In some embodiments, the subject is a mammal, and in some embodiments, the subject is human.

The term “in vivo” refers to an event that takes place in a subject's body.

A. Oxidative Agents

In one aspect, the present invention provides a method of treating a subject suffering a macrophage-related disease, said method comprising administering to a subject in need thereof an effective amount of an oxidative agent. In another aspect, the present invention provides a method of monitoring a treatment with an oxidative agent to a subject suffering from a macrophage related disease. The oxidative agent can be chlorite or compositions comprising chlorite.

I. Chlorite and Other Oxidative Agents

Substances that have the ability to oxidize other substances are typically referred to as oxidative and are known as oxidizing agents, oxidants, or oxidizers, which are used interchangeably herein. An oxidizing agent (also called an oxidant, oxidizer) can be defined as either: a chemical compound that readily transfers oxygen atoms, or a substance that gains electrons in a redox chemical reaction. In both cases, the oxidizing agent becomes reduced in the process. Various common oxidizers contain oxygen (e.g., KClO₄) and can be considered as storage forms of oxygen. Alternatively, the term “oxidizing agent” also includes any time where formal charge is increased (losing electrons), and applies to substances that contain no oxygen, typically halogens comprising fluorine, (F); chlorine, (Cl); bromine, (Br); iodine, (I); and astatine, (At), and substances rich in these elements.

Common oxidizing or oxidative agents that can be used in the methods of the present invention include but are not limited to potassium nitrate (KNO₃), hypochlorite and other hypohalite compounds, iodine and other halogens, chlorite, chlorate, perchlorate, and other analogous halogen compounds, permanganate salts, ammonium cerium(IV) nitrate and related cerium(IV) compounds, hexavalent chromium compounds such as chromic and dichromic acids and chromium trioxide, pyridinium chlorochromate (PCC), and chromate/dichromate compounds; peroxide compounds, Tollens' reagent, sulfoxides, persulfuric acid, ozone, osmium tetroxide (OsO₄), nitric acid, and nitrous oxide (N₂O). The oxidative agent can be non-toxic to monocytes or macrophages at physiologically effective concentrations.

The oxidative agents of the current invention can be compounds that contain both readily-transferable oxygen and halogen atoms, including but not limited to hypochlorite and other hypohalite compounds, chlorite, chlorate, perchlorate and other analogous halogen compounds, and pyridinium chlorochromate (PCC). As used herein, such compounds are referred to as activated-oxygen activated-halogen compounds.

Alternatively, the oxidative agent may be a substance that contains no oxygen, typically halogens comprising fluorine, (F); chlorine, (Cl); bromine, (Br); iodine, (I); and astatine, (At). As used herein, such compounds are referred to non-oxygen activated-halogen compounds.

Many oxidative compounds have demonstrated protective and anti-inflammatory activities, likely due to induction of endogenous defense pathways. For example, metabolites of the stress induced enzyme heme oxygenase 1 (HO-1) such as carbon monoxide (CO) and biliverdin exert potent anti-inflammatory effects (Otterbein L E et al. Nat. Med. 6 (2000) 422-428). The catalytic products of HO-1 including the oxidants CO, Fe2+, and biliverdin are capable of down-regulating inflammatory reactions. Similar cell-protective properties have been described for the redox-active molecule thioredoxin (Hirota K. et al. J. Biol. Chem. 274 (1999) 27891-27897). The use of chlorite to treat various diseases and conditions is described in U.S. Pat. No. 4,725,437; U.S. Pat. No. 4,851,222; McGrath et al., Development of WF10, a novel macrophage-regulating agent, Curr Opin Investig Drugs, 3(3):365-73 (March 2002); U.S. Pat. No. 6,086,922; U.S. Pat. No. 7,105,183; U.S. Pat. No. 8,029,826; U.S. Pat. No. 8,501,244; U.S. Pat. No. 8,231,856; U.S. Pat. No. 8,252,789; U.S. Pat. No. 8,067,035; and U.S. patent application Ser. No. 13/388,411, all of which are incorporated herein by reference in their entirety.

Disclosed herein are compositions and methods for treatment of a subject suffering from a macrophage related disease using chlorite. The chlorite ion is ClO₂⁻. A chlorite (compound) is a compound that contains this group, with chlorine in oxidation state +3. Chlorites are also known as salts of chlorous acid. Chlorine can assume oxidation states of −1, +1, +3, +5, or +7 within the corresponding anions Cl⁻, ClO⁻, ClO₂⁻, ClO₃⁻ or ClO₄⁻ known commonly and respectively as chloride, hypochlorite, chlorite, chlorate, and perchlorate.

II. Tetrachlorodecaoxide (TCDO) and WF10

The present invention also provides methods using one or more chlorite containing agents. The source of chlorite ions for administration of chlorite according to the present invention can be provided in a variety of forms. For example, chlorite can be administered as a chlorite salt, for example, alkali metal salt, e.g. sodium chlorite, potassium chlorite, and the like, or a mixture of chlorite salts, where the chlorite salts are preferably pharmaceutically acceptable. In addition or alternatively, chlorite can be administered as a matrix of chlorite ions, e.g., described in U.S. Pat. No. 4,507,285. In one embodiment, the chlorite ions as provided in a composition having the general formula:

ClO₂⁻x nO₂

wherein “n” can be a value of about 0.1-0.25. Such agents can have an O₂ band at 1562 cm⁻¹ in the Raman spectrum and an O—O interval of 123 pm. Production of such agents is known in the art, see e.g., U.S. Pat. No. 4,507,285.

In one embodiment, the method of treatment involves administration of a liquid composition comprising an aqueous solution of a product known as “tetrachlorodecaoxygen anion complex”, commonly known as TCDO. Production of TCDO is well known, see e.g., Example 1 of U.S. Pat. No. 4,507,285. In some embodiments, the chlorite containing agents that can be used in the methods of the present invention for treating diabetes or related disorders include but are not limited to chlorite salt, such as alkali metal salt, sodium chlorite, potassium chlorite, and the like, a matrix of chlorite salts, a matrix of chlorite ions, e.g., compositions having the general formula ClO₂xnO₂, where “n” can be a value or about 0.1-0.25. One example is TCDO. One of the aqueous TCDO formulations is WF10. WF10 is an aqueous formulation of the drug OXO-K993. Oxoferin is a topical formulation of the same drug and is registered and marketed as a wound healing agent in Europe and Asia. WF10 is a sterile, pyrogen-free, aqueous 10% (w/v) solution of OXO-K993 with no additional inactive ingredients and is intended for intravenous infusion. TCDO is analytically characterized as a solution containing 4.25% chlorite, 1.9% chloride, 1.5% chlorate, 0.7% sulfate, and sodium as the cation. The active principle is defined by the chlorite ion content. In one embodiment, WF10 solution contains about 63 mmol/1 of chlorite.

Tetrachlorodecaoxide (TCDO) is a chlorite-containing drug used for the dressing of wounds, immunomodulation and as radiation protective agent. Due to its oxidizing properties, TCDO can destroy most pathogens although it is not regarded as antibiotic. But the main reason for its use for dressing of wounds is not its bactericidal activity. This drug is regarded as immunomodulating, that is, it acts by stimulating the immune system of the body. Tetrachlorodecaoxide combines with the heme part of hemoglobin, myoglobin and peroxidase, forming a TCDO-hemo complex. This in turn activates the macrophages and accelerates the process of phagocytosis which engulfs most of the pathogens and cell debris present on the surface of the wound, thus cleaning the wound surface and helping in the regenerative process. Tetrachlorodecaoxide is also mitogenic and chemotactic. The mitogenic impulse gives rise to two factors, MDGF (Macrophage derived growth factor) and WAF (Wound angiogenesis factor). The MDGF deposits fibroblasts and synthesizes collagen fibers, which fill the gap in the wounds, the WAF helps in the formation of new capillaries which further enhances the healing process. The chemotactic impulse acts on the myocyte (muscle cell) and causes it to contract, thereby bringing the wound edges closer and reducing the wound surface. Simultaneous influence of all these factors accelerates the wound healing with minimal scarring.

WF10 is a 1:10 dilution of tetrachlorodecaoxide (TCDO) formulated for intravenous injection. WF10 specifically targets macrophages. WF10 potentially modulates disease-related up-regulation of immune responses both in vitro and in vivo. Thus immune response is influenced in a way that inappropriate inflammatory reactions are downregulated (Arzneimittelforschung. 2001; 51(7):554-62. Schempp H, et al). WF10 is currently being studied for treatment of late-stage HIV disease, as well as recurrent prostate cancer, late post-radiation cystitis, autoimmune disease and chronic active hepatitis C disease. WF10 is approved for use in Thailand under the name IMMUNOKINE in patients with post-radiation chronic inflammatory disease including cystitis, proctitis and mucositis.

In vivo studies have investigated the effects of WF10 on monocytes, macrophages and lymphocytes, on humoral and cellular immunity, and on response to local or total body irradiation (reviewed by McGrath M S et al. Current Opinion in Investigational Drugs 2002 3(3)). WF10 increased the number of macrophages infiltrating a skin blister in a human wound healing model (Hansel M et al. Skin Pharmacol 1988 1:64). In rats, WF10 increased the proportion of granulocytes, peripheral blood monocytes (PBMCs) and large granular lymphocytes (LGLs), and stimulated erythropoiesis after total body X-irradiation (Ivankovic S et al. OXO Study Report 1988 March; Ivankovic S et al. Radiat Res 1988 115: 115-123). In mice, WF10 stimulated regeneration of hematopoietic stem cells receiving sublethal doses of J-irradiation (Mason K A et al. Radiat Res 1993 136: 229-235). In other studies, WF10 displayed direct antitumor effects against radiation-induced, hemical-induced and metastatic malignant and benign tumors (Kempf S R et al. International Symposium on Tissue Repair 1990 Thailand; Milas L. OXO Study Report 1991 September; Kempf S R et al. Radiat Res 1994 139: 226-231). WF10 altered proportions of T-helper and suppressor/cytotoxic cells in spleen and thymus and increased both the humoral and cellular immune responses (Gillissen G et al. OXO Study Report 1993).

Without being bound by theory, it has been suggested that WF10 causes marked inhibition of inducible genes related to T-cell proliferation and cause reproducible up-regulation of inflammatory gene expression in macrophages in vitro, which is thought to contribute to the higher rate of apoptosis in activated macrophages. These data, coupled with an earlier report of WF10 inhibition of T-cell activation (McGrath M S et al. Transplant Proc. 1998 30: 4200-4202), show that WF10 causes profound changes in T-cell function through regulation of macrophage activation. The WF10 oxygen/chlorite matrix is stable until interaction with heme-associated iron, whereupon it is converted to an active chlorite molecule through a Michealis-Menten reaction and intermediate production of a reactive compound I. Chlorite is the active form of the drug thought to mediate the immunological effects in macrophages.

A dose-ranging clinical study was conducted from 1993 to 1994 in 44 HIV-positive patients with <500 CD4+ T cells/mm (Raffanti S P et al. Infection 1998 26: 201-206). The study established the maximum tolerated dose as 0.5 ml/kg/day of WF10, when administered in four 5-day cycles, with each cycle followed by 16 days of without treatment. No significant adverse events or clinical laboratory toxicity were observed at this dosage. Plasma CD8+ T-cell counts increased in a dose-dependent manner over four cycles of WF10 administration. This study demonstrated that WF10 at a dose of 0.5 ml/kg was associated with a sustained immunological response, i.e., sustained elevation of CD8+ T cell numbers, consistent with the proposed mechanism of action. Furthermore, a single-center, phase I/II study, was conducted in 1997 to evaluate safety and the effects of WF10 on the kinetics of red blood cell (RBC) survival, selective immunological markers of HIV disease, macrophage activation and viral kinetics (Hemdier B et al. Keystone Symposia on Molecular and Cellular Biology. 1998). Changes in immunological parameters of cells from HIV+ patients in response to WF10 treatment are summarized in Table 1 in McGrath M S et al. Current Opinion in Investigational Drugs 2002 3(3), including an increase in CD3+CD4+ cells, an increase in CD3+ CD8+ cells, an increase in CD3+ CD4+ CD38− cells, an increase in CD3+ CD8+ CD38− cells, an increase in CD3+ CD8+ CD28− cells, a decrease in CD3+ CD8+ CD28+ cells, a decrease in CD3+ CD4+ CD38+ cells, a decrease in all CD14+ cells, and a decrease in CD20+ HLR-DR+ cells. The results suggested that WF10 reduced antigen presentation while concurrently inducing phagocytosis in macrophages with impaired function. WF10 had no effect on HIV load over the course of the trial. No significant differences were detected between the WF10 and placebo group in hematological and blood chemistry values, including parameters specifically associated with hemolysis.

As appropriate, agents that provide a source of chlorite ions can be administered in a free base or free acid form, i.e., as the free compound and not as a salt. In some embodiments, the chlorite formulation contains about 150 μM chlorite.

Additionally, any pharmaceutically acceptable salt(s) of the compound(s) can also be used. Pharmaceutically acceptable salts are those salts which retain the biological activity of the free compounds and which are not biologically or otherwise undesirable. As appropriate, stereoisomers of the compounds disclosed can also be used in the invention, including diastereomers and enantiomers, as well as mixtures of stereoisomers, including but not limited to racemic mixtures. Unless stereochemistry is explicitly indicated in a structure, the structure is intended to embrace all possible stereoisomers of the compound depicted.

The oxidative compound or chlorite as described herein can be WF10. WF10 is a chlorite-based compound. After interaction with heme proteins, the chlorite matrix of WF10 acquires oxidizing and chlorinating properties (Schempp H. et al. 1999). It has been suggested that WF10 exerts potent immunomodulatory effects most likely through generating physiologic oxidative compounds namely chloramines. Chloramines have been reported to exert cell-protective and anti-inflammatory activities (Choray M. et al. Amino Acids 23 (2002) 407-413).

Pro-oxidative substances can also have a direct effect on transcriptional activities of the NFAT species of transcription factors. The nuclear translocation of NFAT requires their dephosphorylation by the calcium/calmodulin dependent serine/threonine phosphatase calcineurin. The phosphatase activity of calcineurin is redox sensitive. WF10 is able to inhibit antigen receptor driven lymphocyte proliferation. Expression of NFAT regulated genes is strongly suppressed by WF10, and the nuclear translocation of NFATc is inhibited. The WF10 associated inhibition of NFAT regulated genes in activated T cells, in concert with the induction of several monocyte associated pro-inflammatory genes, suggest activation of the innate myeloid functions concomitant with the inactivation of adaptive proliferative lymphocyte response. This approach represents a novel method of targeting redox-regulation for the treatment of inflammatory disorders. In some embodiments, the macrophage related diseases that can be treated using the methods of the present invention are inflammatory diseases.

III. Chlorite Purity and pH

Methods of formulating chlorite have been described in US Patent Pub. No. 20070145328, filed Dec. 21, 2006 and entitled “Chlorite Formulations, and Methods of Preparation and Use Thereof,” which is incorporated herein by reference in its entirety. Such formulations are suitable for various modes of administration, including but not limited to non-topical, parenteral, systemic, or intravenous administration.

Described in present invention are compositions and methods using chlorite formulated in aqueous solution in which the chlorite is greater than 95% pure. In some cases, the chlorite can be greater than 97%, 99%, 99.5% or 99.9% pure. In some cases, the chlorite can be at least 95%, 97%, 99%, 99.5% or 99.9% pure. As used herein, the “purity” of chlorite in a sample is calculated as the percent weight of chlorite salt to the total weight of the sample. In determining the purity of chlorite in a solution, the weight of the solvent (e.g., water in an aqueous solution) is not included. Purity may be evaluated using ion chromatography and an ion detector, by calibrated integration of the respective peaks; for example, chlorite, chloride, chlorate, phosphate and sulfate in the compound or formulation. For example, chlorite is commercially available as sodium chlorite, technical grade, at a purity of 80% (catalog No. 244155 Sigma-Aldrich).

Alternatively, crystalline sodium chlorite is provided in a purity greater than 95%, greater than 96%, greater than 97%, greater than 98%, greater than 99%, greater than 99.5% or greater than 99.9%. Solid pharmaceutical formulations comprising crystalline sodium chlorite in a purity greater than 95%, greater than 96%, greater than 97%, greater than 98%, greater than 99%, greater than 99.5% or greater than 99.9% in addition to one or more pharmaceutical excipients are also encompassed.

The chlorite formulations for use with the present invention can comprise low amounts of chlorate, sulfate or chloride. As used herein, a formulation is “substantially free” of a molecule if the molecule comprises no more than 1 part in 1000 per weight of non-solvent molecules in the formulation. In certain embodiments, the weight ratio of chlorite to chlorate is greater than 100:1.5, greater than 100:0.5, greater than 100:1, or greater than 100:0.1. In one embodiment, the composition is substantially free of chlorate. In another embodiment, the weight ratio of chlorite to chloride is greater than 100:45.5 or greater than 100:8.5. In one embodiment the composition is substantially free of chloride. In a further embodiment, the weight ratio of chlorite to sulfate is greater than 100:16.4 or greater than 100:1.6. In one embodiment the composition is substantially free of sulfate.

The pH of a chlorite formulation for use with the present invention can be adjusted to between about 7 and about 11.5. In some embodiments, the pH of a chlorite formulation is lowered to between about 7 and about 11.5 using a pH adjusting compound that does not expose the formulation to high local acidity. In some embodiments, the pH adjusting compound is any one or more of monosodium phosphate, disodium phosphate, or acetic acid.

Also described herein are methods of preparing chlorite formulations and pharmaceutical formulations, including but not limited to the chlorite formulations specifically described herein. Also described herein are kits and methods of administration of the formulations and pharmaceutical formulations described herein. Various exemplary aspects and variations of the invention are described in the “Brief Summary of the Invention,” as well as elsewhere herein, including but not limited to the Examples. It is also understood that the invention includes embodiments comprising, consisting essentially of, and/or consisting of one or more elements as described herein.

In some embodiments, the invention makes use of aqueous formulations comprising chlorite. In some embodiments, the chlorite formulation comprises an aqueous solvent, and optionally one or more other solvents for chlorite. In some embodiments, the formulations comprise chlorite and an aqueous solvent for chlorite, and have a pH of about 7 to about 11.5.

Solvents or combinations of solvents for use in the formulations described herein can be determined by a variety of methods known in the art. One non-limiting example includes (1) theoretically estimating solvent solubility parameter value(s) and choosing the one(s) that match with chlorite, using standard equations in the field; and (2) experimentally determining the saturation solubility of chlorite in the solvent(s), and (3) choosing one or more that exhibits the desired solubility, and (4) selecting a solvent or solvents that do not diminish the activity of chlorite, or that do not or only minimally react with chlorite. In some embodiments, the liquid formulations described herein comprise a plurality of solvents.

In some embodiments, the chlorite formulations comprise an aqueous solvent. In some variations, water is the principal solvent in the aqueous formulations. In some variations, water is at least about 50% by volume of the solvent component of an aqueous formulation. In some variations, water is at least about 50% by volume of the aqueous formulation. In some variations, water is any of between about 50 to about 60, between about 60 to about 70, between about 70 to about 80, between about 80 to about 90, between about 90 to about 99, at least about 50, at least about 60, at least about 70, at least about 80, at least about 90, or at least about 95, about 50, about 60, about 70, about 80, about 90, or about 95 percent by volume of the solvent component. In some variations, water is any of between about 50 to about 60, between about 60 to about 70, between about 70 to about 80, between about 80 to about 90, between about 90 to about 99, at least about 50, at least about 60, at least about 70, at least about 80, at least about 90, or at least about 95, percent by volume of the aqueous formulation. In some variations, water is at least about 95% by volume of the aqueous formulation. In some variations, water is between about 80 to about 90% by volume of the aqueous formulation. In some variations, water is between about 90 to about 99% by volume of the aqueous formulation.

The formulations may have differing concentration of chlorite. In some embodiments, the concentration of chlorite in the formulation is high, and then is diluted to a less concentrated form prior to administration. In some embodiments, a formulation described herein is diluted about, at least about or less than about 2.5×, about 5×, about 7.5×, about 10×, about 20×, about 25×, about 50×, about 100×, about 200×, about 250×, about 300×, about 500×, or about 1000×. In some embodiments, a formulation described herein is diluted between about 2× and about 10×, between about 10× and about 50×, between about 50× and about 100×, between about 100× and about 500×, or between about 500× and about 1000×. In some embodiments, a formulation as described herein is diluted between about 2× and about 10×. In some embodiments, a formulation as described herein is diluted between about 10× and about 50×. In some embodiments, a formulation as described herein is diluted about 7.5×. In some embodiments, a formulation as described herein is diluted about 25×. In some embodiments, a formulation as described herein is diluted about 200×.

In some embodiments, the concentration of chlorite in the formulations described herein is between about 1 μM and about 1.5 M. In another embodiments, the concentration of chlorite in the formulations described herein is between any of about 1 M and about 1.5 M; between about 1 μM and about 100 mM; between about between about 10 μM and about 100 mM; between about 0.1 mM and about 10 mM; between about 0.1 mM and about 500 mM; between about 0.1 mM and about 200 mM; between about 1 mM and about 100 mM; between about 0.1 mM and about 5 mM; between about 50 mM and about 100 mM; between about 55 mM and about 70 mM; between about 60 mM and about 65 mM; between about 100 mM and about 500 mM; between about 200 mM and about 400 mM; between about 300 mM and about 700 mM; about 1 mM; about 1.5 mM; about 2 mM; about 2.5 mM; about 3 mM; about 3.5 mM; about 4 mM; about 5 mM; about 10 mM; about 20 mM; about 30 mM; about 40 mM; about 50 mM; about 60 mM; about 62 mM; about 65 mM; about 70 mM; about 80 mM; about 90 mM; about 100 mM; at least about 0.1 mM; at least about 1 mM; at least about 2 mM; at least about 5 mM; at least about 10 mM; at least about 20 mM; at least about 30 mM; at least about 40 mM; at least about 50 mM; at least about 60 mM; at least about 70 mM; at least about 80 mM; at least about 90 mM; or at least about 100 mM. In preferred embodiments, the concentration of chlorite in the formulations described herein is about or at least about 60 mM.

In some embodiments, the concentration of chlorate in the formulations described herein is between about 50 mM and about 100 mM. In some embodiments, the concentration of chlorate in the formulations described herein is between about 55 mM and about 75 mM. In some embodiments, the concentration of chlorate in the formulations described herein is between about 0.1 mM and about 10 mM. In some embodiments, the concentration of chlorate in the formulations described herein is between about 1 mM and about 5 mM.

In some embodiments, the chlorite formulation has a pH no greater than about 12.0. In some embodiments, the pH of the formulation is any of no greater than about 11.5, about 11.0, about 10.5, about 10.0, about 9.5, about 9.0, about 8.5, about 8.0, about 7.5, about 7.0, about 6.5, or about 6.0. In some embodiments, the pH of the formulation is no greater than about 11.5. In some embodiments, the pH of the formulation is no greater than about 10.5. In some embodiments, the pH of the formulation is no greater than about 8.5. In some embodiments, the pH of the formulation is no greater than about 7.5. In some embodiments, the pH of the formulation is between any one or more of about 7 and about 12; between about 7 and about 11.5; between about 7 and about 10.5; between about 7 and about 10; between about 7 and about 9.5; between about 7 and about 9.0; between about 7 and about 8.5; between about 7 and about 8.0; between about 7 and about 7.5; between about 7.5 and about 8; between about 7.5 and about 8.5; between about 7 and about 8; between about 8 and about 9; between about 7.0 and about 8.5; between about 8 and about 8.5; between about 8.5 and about 9; between about 7.1 and about 7.7; between about 7.2 and about 7.6; between about 7.3 and about 7.4; about 7.0; about 7.1; about 7.2; about 7.3; about 7.4; about 7.5; about 7.6; about 7.7; about 7.8; about 7.9; about 8.0; about 8.1; about 8.2; about 8.3; about 8.4; about 8.5; about 8.6; about 8.7; about 8.8; or about 8.9. In some embodiments, the chlorite formulation has a pH of about 7.0 to about 9.0. In some embodiments, the chlorite formulation has a pH of about 7.0 to about 8.5. In some embodiments, the chlorite formulation has a pH of about 6.0 to about 8.5. In some embodiments, the chlorite formulation has a pH of about 7.0 to about 8.0. In some embodiments, the chlorite formulation has a pH of about 7.4. The chlorite formulation can have a pH that is at a physiological level.

In some embodiments, the chlorite formulations have a pH as described above, and are formulated for any one or more of parenteral, systemic, or intravenous administration. In some embodiments, the chlorite formulations have a pH as described above, and have a percentage chlorite purity as described herein.

In some embodiments, the formulations described herein have a pH as described above, and have a concentration of chlorite as described herein. In some embodiments, the aqueous formulations described herein have a pH between about 7 and about 11.5, or between about 7.0 and about 10, or between about 7.0 and about 9.0, or between about 7.0 and about 8.5, or between about 7.1 and about 7.7, and have a concentration of chlorite between about 1 and about 100 mM. In some embodiments, the aqueous formulations described herein have a pH between about 7 and about 11.5, or between about 7.0 and about 10, or between about 7.0 and about 9.0, or between about 7.0 and about 8.5, or between about 7.1 and about 7.7, and have a concentration of chlorite between about 1 and about 5 mM. In some embodiments, the aqueous formulations described herein have a pH between about 7 and about 11.5, or between about 7.0 and about 10, or between about 7.0 and about 9.0, or between about 7.0 and about 8.5, or between about 7.1 and about 7.7, and have a concentration of chlorite between about 50 and about 80 mM.

In some embodiments, the aqueous formulations described herein have a pH between about 7 and about 11.5, or between about 7.0 and about 10, or between about 7.0 and about 9.0, or between about 7.0 and about 8.5, or between about 7.1 and about 7.7, wherein the pH was adjusted with a pH adjusting agent that is any one or more of a phosphate, or acetic acid.

In some embodiments, the formulations described herein are stable with respect to one or more of pH or chlorite degradation over a period of any of at least about 1 day, at least about 2 days, at least about 3 days, at least about 4 days, at least about 5 days, at least about 6 days, at least about 1 week, at least about 2 weeks, at least about 3 weeks, at least about 4 weeks, at least about 5 weeks, at least about 6 weeks, at least about 7 weeks, at least about 8 weeks, at least about 1 month, at least about 2 months, at least about 3 months, at least about 4 months, at least about 5 months, or at least about 6 months. In some embodiments, the formulations described herein are stable with respect to one or more of pH or chlorite degradation over a period of any of at least about 1 week. In some embodiments, the formulations are stable with respect to one or more of pH or chlorite degradation over a period of any of at least about 1 month. In some embodiments, the formulations described herein are stable with respect to one or more of pH or chlorite degradation at one or more of room temperature, refrigerated conditions, or approximately 4 degree C. In some embodiments, the formulations described herein are stable with respect to one or more of pH or chlorite degradation under conditions of diminished light or storage in a container that limits the amount of light to which the formulation is subjected. In some embodiments, the formulations described herein are stable with respect to one or more of pH or chlorite degradation when stored in the dark. Examples of stable pH, as used herein, means that the pH of the formulation changes by less than any of about 0.1, about 0.2, about 0.3, about 0.4, about 0.5, about 0.6, about 0.7, about 0.8, about 0.9, or about 1 relative to the pH of the formulation as initially prepared. In some embodiments, the pH of the formulation changes by less than about 0.2 relative to the pH of the formulation as initially prepared. The pH may be measured using, for example, a pH meter. Examples of stable chlorite formulations include those in which less than any of about 0.1%, less than about 0.2%, less than about 0.3%, less than about 0.4%, less than about 0.5%, less than about 0.6%, less than about 0.7%, less than about 0.8%, less than about 0.9%, less than about 1%, less than about 2%, less than about 3%, less than about 4%, less than about 5%, less than about 6%, less than about 7%, less than about 8%, less than about 9%, or less than about 10% of the chlorite degrades into a non-chlorite ion relative to the amount of chlorite present in the formulation as initially prepared. In some embodiments, less than about 2% of the chlorite degrades into a non-chlorite compound relative to the amount of chlorite present in the formulation as initially prepared. In some embodiments, less than about 0.5% of the chlorite degrades into a non-chlorite compound relative to the amount of chlorite present in the formulation as initially prepared. The presence of non-chlorite elements may be measured, for example, using gas chromatography (GC), mass spectrometry, or other methods known by those of skill in the art.

In some embodiments, the chlorite formulations described herein comprise no greater than about 5% by weight of deleterious non-chlorite elements of other commercially available formulations. In some embodiments, the chlorite formulations described herein comprise any of no greater than about 4%, about 3%, about 2%, about 1%, about 0.5%, about 0.3%, about 0.25%, about 0.2%, about 0.1%, about 0.05%, or about 0.02%, by weight of deleterious non-chlorite elements of other commercially available formulations. In some embodiments, the chlorite formulations described herein comprise any of no greater than about 4% by weight of deleterious non-chlorite elements of other commercially available formulations. In some embodiments, the chlorite formulations described herein comprise any of no greater than about 2% by weight of deleterious non-chlorite elements of other commercially available formulations. In some embodiments, the chlorite formulations described herein comprise any of no greater than about 0.5% by weight of deleterious non-chlorite elements of other commercially available formulations. In some embodiments, the chlorite formulations described herein comprise any of no greater than about 0.05% by weight of deleterious non-chlorite elements of other commercially available formulations. In some embodiments, the chlorite formulations described herein are substantially free of the deleterious non-chlorite elements of other commercially available formulations. Non-limiting examples of methods of detection of non-chlorite components include HPLC; SPCS, for example using a Novosep A2 column with 3.6 mM Sodium Carbonate as a mobile phase, 5μ, 250×4.0 mm, flow rate 0.8 mL/min; DS-Plus Suppressor, for example using a Novosep A2 column with 3.6 mM Sodium Carbonate as a mobile phase, 5μ, 250×4.0 mm, flow rate 0 8 mL/min; an Allsep A-2 Anion column using 2.1 mM NaHCO.sub.3/1.6 mM Na.sub.2CO₃ as a mobile phase, 100×4.6 mm, flow rate 2.0 mL/min; an anion HC column using 2.8 mM NaHCO.sub.3:2.2 mM Na₂CO₃ in 10% Methanol as a mobile phase, 150×4.6 mm, flow rate 1.4 mL/min; or an Allsep A-2 Anion column using 2.1 mM NaHCO₃/1.6 mM Na₂CO₃ as a mobile phase, 5μ, 100×4.6 mm, flow rate 1.0 mL/min. See, for example, the Alltech Associates, Inc. Grace Davison line of products and product information for details. In some embodiments, formulations described herein comprise no greater than about 10%, about 20%, about 30%, about 40%, about 50%, about 60%, about 70%, about 75%, about 80%, about 85%, about 90%, or about 95% of the amount of a member of the group consisting of, or alternatively any one or more of, chlorate ions and sulfate ions present in an equal weight/volume percent of chlorite formulated as WF10 or a dilution thereof. That is, in some embodiments, when a non-WF10 formulation as described herein comprises a certain percent w/v of chlorite, such formulation has no greater than about the stated percentage of the amount of one or more of the specified non-chlorite components in WF10 or a dilution thereof, wherein the WF10 or dilution thereof comprises the same percent w/v of chlorite as is found in the non-WF10 formulation with which it is being compared. In some embodiments, the formulations described herein comprise no greater than about 75% of the amount of a member of the group consisting of, or alternatively any one or more of, chlorate ions and sulfate ions present in an equal weight/volume percent of chlorite formulated as WF10. In some embodiments, the formulations described herein comprise no greater than about 85% of the amount of a member of the group consisting of, or alternatively any one or more of, chlorate ions and sulfate ions present in an equal weight/volume percent of chlorite formulated as WF10. In some embodiments, the formulations described herein comprise no greater than about 50% of the amount of a member of the group consisting of, or alternatively any one or more of, chlorate ions and sulfate ions present in an equal weight/volume percent of chlorite formulated as WF10.

It can be understood from the product insert of WF10 that WF10 reportedly includes a ratio of chlorite to chlorate of 100:35.7 (4.25% to 1.5%), a ratio of chlorite to chloride of 100:45.5 (4.25% to 1.9%) and a chlorite to sulfate ratio of 100:16.4 (4.25% to 0.7%).

Examples of deleterious non-chlorite components include non-chlorite components that cause an adverse reaction when administered to physiological systems. In some variations, a deleterious non chlorite component is associated with one or more indicia of toxicity in one or more of in vitro or in vivo assays known in the art, or are associated with one or indicia of toxicity when administered to a physiological system, including but not limited to a subject, including but not limited to a human subject. Deleterious non chlorite components include but are not limited to sulfate, chlorine dioxide, chlorate, and borate. In some embodiments, the chlorite formulations described herein are substantially free of the deleterious non-chlorite elements of WF10. In some variations, the chlorite formulations described herein are substantially free of sulfate and chlorate ions.

In some embodiments, the chlorite formulations described herein contain less than about 1.9% of chloride ions. In some embodiments, the chlorite formulation contains any of less than about 1.9%, less than about 1.8%; less than about 1.5%; less than about 1.0%; less than about 0.5%; less than about 0.3%; less than about 0.1%; less than about 0.05%; less than about 0.01%; less than about 0.001%; between about 0.001 to about 0.1%; between about 0.1 to about 0.5%; between about 0.5 to about 1.0%; between about 1.0 to about 1.5%; or between about 1.5 to about 1.8% by weight of chloride ions. In some embodiments, the chlorite formulation contains less than about 0.5% by weight of chloride ions. In some embodiments, the chlorite formulation contains less than about 0.24% by weight of chloride ions. In some embodiments, the chlorite formulation contains less than about 0.2% by weight of chloride ions. In some embodiments, the chlorite formulation contains less than about 0.1% by weight of chloride ions. In some embodiments, the chlorite formulation is substantially free of chloride ions. In some embodiments, the level of chloride ions is below the level of detection using HPLC.

In some embodiments, the chlorite formulation contains less than about 1.5% of chlorate ions. In some embodiments, the chlorite formulation contains any of less than about 1.4%, less than about 1.3%; less than about 1.0%; less than about 0.5%; less than about 0.3%; less than about 0.1%; less than about 0.01%; less than about 0.001%; between about 0.001 to about 0.1%; between about 0.001 to about 0.01%; between about 0.01 to about 0.1%; between about 0.1 to about 0.5%; between about 0.5 to about 1.0%; or between about 1.0 to about 1.4% of chlorate ions. In some embodiments, the chlorite formulation is substantially free of chlorate ions. In some embodiments, the chlorite formulation contains less than about 0.5% by weight of chlorate ions. In some variations, the chlorite formulation is substantially free of chlorate ions. In some embodiments, the chlorite formulation contains less than about 0.19% by weight of chlorate ions. In some embodiments, the chlorite formulation contains less than about 0.1% by weight of chlorate ions. In some embodiments, the level of chlorate ions is below the level of detection using HPLC.

In some embodiments, the chlorite formulation contains less than about 0.7% of sulfate ions. In some embodiments, the chlorite formulation contains any of less than about 0.65%; less than about 0.6%; less than about 0.5%; less than about 0.4%; less than about 0.3%; less than about 0.2%; less than about 0.1%; less than about 0.08%; less than about 0.07%; less than about 0.06%; less than about 0.05%; less than about 0.005%; less than about 0.0005%; between about 0.001 to about 0.1%; between about 0.01 to about 0.1%; between about 0.01 to about 0.5%; between about 0.06 to about 0.08%; or between about 0.5 to about 0.65% of sulfate ions. In some embodiments, the chlorite formulation contains between about 0.5 to about 0.65% of sulfate ions. In some embodiments, the chlorite formulation is substantially free of sulfate ions. In some embodiments, the chlorite formulation contains less than about 0.5% by weight of sulfate ions. In some embodiments, the chlorite formulation is substantially free of sulfate ions. In some embodiments, the chlorite formulation contains less than about 0.08% by weight of sulfate ions. In some embodiments, the level of sulfate ions is below the level of detection using HPLC.

In some embodiments, the chlorite formulations described herein comprise phosphate ions. In some embodiments, the chlorite formulations described herein comprise sodium ions. In some embodiments, a chlorite formulation comprises chlorite, an aqueous solvent, sodium, and phosphate ions. In some variations, the aqueous solvent consists essentially of water. In some embodiments, a chlorite formulation consists essentially of chlorite, water, sodium, and phosphate, and is substantially free of chlorate. In some embodiments, a chlorite formulation consists essentially of chlorite, water, sodium, and phosphate, and is substantially free of chlorate, and further comprises a pharmaceutically acceptable diluent. In some embodiments, sodium and phosphate are provided in whole or in part as monosodium phosphate or disodium phosphate. In some embodiments, the pharmaceutically acceptable diluent is a saline solution.

In some embodiments, the chlorite formulations described herein comprise no greater than about 10% by weight of by products or impurities present in commercially available technical grade chlorite. Non-limiting examples of by-products or impurities present in commercially available technical grade chlorite include chlorate, sulfate, chlorine dioxide, chloride, sodium bicarbonate, and sodium carbonate. In some embodiments, the chlorite formulations described herein comprise no greater than about any of 15%, about 12%, about 10%, about 9%, about 8%, about 7%, about 6%, about 5%, about 4%, about 3%, about 2%, about 1%, about 0.5%, about 0.3%, about 0.1%, between about 0.1 to about 5%; between about 5 to about 10%; or between about 10 to about 15% by weight of one or more degradation products or impurities present in commercially available technical grade chlorite, including but not limited to one or more of chlorate or sulfate. In some embodiments, the chlorite formulations described herein comprise no greater than about 0.5% by weight of degradation products or impurities present in commercially available technical grade chlorite, including but not limited to one or more of chlorate or sulfate. In some embodiments, the chlorite formulations described herein comprise no greater than about 5% by weight of degradation products or impurities present in commercially available technical grade chlorite, including but not limited to one or more of chlorate or sulfate. In some embodiments, the chlorite formulations described herein are substantially free of the degradation products or impurities present in commercially available technical grade chlorite, including but not limited to chlorate or sulfate.

In some embodiments, the formulations described herein are less toxic to a subject than previously reported chlorite formulations at the same concentration of chlorite, when administered by at least one of the routes of administration described herein, including but not limited to by non-topical, systemic, parenteral, or intravenous administration. In some embodiments, the toxicity of a chlorite formulation is analyzed for toxicity using an in vivo or in vitro toxicity assay, including well-known toxicity assays. In some embodiments, the chlorite formulation is analyzed for toxicity using a non-specific in vitro toxicity assay.

In another variation, toxicity is measured according to various response indicia of toxicity in a subject after administration of the chlorite formulations described herein, as compared to administration of other commercially available chlorite formulations. In some variations, toxicity is measured relative to systemic administration of chlorite formulated as WF10. In another variation, toxicity is measured relative to intravenous administration of chlorite formulated as WF10 to a subject. In some variations, toxicity is measured after administration to a mammalian subject, including but not limited to a human subject. In some variations, toxicity is measured as one or more of irritation to the surface to which the chlorite formulation is exposed, including but not limited to the gastrointestinal tract, nausea, vomiting, diarrhea, abdominal pain, hemolysis, methemoglobinemia, cyanosis, anuria, coma, convulsions, liver damage, kidney damage, loss of appetite, or weight loss. In some variations, toxicity is measured as one or more of asthenia, injection site pain, headache, rhinitis, or diarrhea. See, e.g., McGrath M S, “Development of WF10, A Novel Macrophage-Regulating Agent,” Curr Opin Investig Drugs, 3(3):365-73 (March 2002), which is incorporated by reference in its entirety. In another variation, toxicity is measured as anemia. See, e.g., Kempf et al., “Comparative Study on the Effects of Chlorite Oxygen Reaction Product TCDO (Tetrachlorodecaoxygen) and Sodium Chlorite Solution (NaClO2) With Equimolar Chlorite Content on Bone Marrow and Peripheral Blood of BDIX Rats,” Drugs Under Experimental and Clinical Research, 19(4):165-1 (1993). In some variations, toxicity is measured as asthenia. In some variations, toxicity is measured as injection site reaction. In some variations, toxicity is measured as injection site pain.

IV. Methods of Adjusting the pH of Formulations Sensitive to pH

Various methods can be used to adjust the pH of formulations and pharmaceutical formulations comprising chlorite. It is intended that the methods described herein can be used to produce the formulations or pharmaceutical formulations described herein for use with the present invention. However, the formulations and pharmaceutical formulations described herein may also be produced by other methods, and the formulations and pharmaceutical formulations described herein are not limited to those produced by the methods described herein.

Some compounds or formulations are sensitive to high local acidity or alkalinity, requiring proper methods to adjust the pH of such compounds or formulations. Preferred pH adjusting agent(s) or pH adjusting compound(s) are weak acids or weak bases having a pKa of about 4 to about 9, a pKa of about 5 to about 9, or a pKa of about 5 to about 8, or a pKa of about 6 to about 7.5. Examples include, but are not limited to a phosphate buffer having a pKa of about 4 to about 9 as well known in the field, for example, monobasic phosphates, or monosodium phosphate and/or disodium phosphate and lower alkanoic acids, for example, acetic acid or propionic acid. In some embodiments, the pH of a formulation sensitive to acidity is lowered to between about 7 and about 11.5 using a pH adjusting compound that does not expose the formulation to acidity, including but not limited to a high local acidity in the area around the pH adjusting compound. In some embodiments, the pH of a formulation sensitive to acidity is lowered to between about 7 and about 10 using a pH adjusting compound that does not expose the formulation to acidity, including but not limited to a high local acidity in the area around the pH adjusting compound. In some embodiments, the pH of a formulation sensitive to acidity is lowered to between about 7 and about 9.5 using a pH adjusting compound that does not expose the formulation to acidity, including but not limited to a high local acidity in the area around the pH adjusting compound. In some embodiments, the pH of a formulation sensitive to acidity is lowered to between about 7 and about 9.0 using a pH adjusting compound that does not expose the formulation to acidity, including but not limited to a high local acidity in the area around the pH adjusting compound. In some embodiments, the pH of a formulation sensitive to acidity is lowered to between about 7 and about 8.5 using a pH adjusting compound that does not expose the formulation to acidity, including but not limited to a high local acidity in the area around the pH adjusting compound. In some embodiments, the pH of a formulation sensitive to acidity is lowered to between about 7.1 and about 7.7 using a pH adjusting compound that does not expose the formulation to acidity, including but not limited to a high local acidity in the area around the pH adjusting compound.

“High local acidity,” as used herein, refers to the pKa of one or more molecules local to a chlorite molecule, as opposed to the overall acidity of a solution as would be measured, for example, using a pH meter. To determine whether a pH-adjusting agent will subject chlorite to high local acidity, the pKa of the pH adjusting agent can be identified using, for example, the CRC Handbook of Chemistry and Physics (86th Edition, David R. Lide ed., CRC Press, 2005).

Lowering the pH of chlorite formulations has been challenging because many pH adjusting agents expose compounds or formulations to high acidity in the local area of the molecules of the pH-adjusting compound. In the presence of high local acidity, some amount of non-chlorite compounds are generated, e.g., chlorate and/or chlorine dioxide. See, e.g., Ullmann's Encyclopedia of Industrial Chemistry, Vol. A6, Ed. Wolfgang Gerhartz, 5th Ed. (1986), which is incorporated herein by reference in its entirety. Such degradation products may not be desired in formulations for parenteral or systemic administration to physiological systems, e.g., because they are not inactive in physiological systems. Some such degradation products result in toxicity, including but not limited to the toxicities, including but not limited to non-specific toxicity, described herein.

Unless the context makes clear, the pH of any of the formulations or pharmaceutical formulations described herein may be adjusted using the methods described herein.

In some variations, the activity of a therapeutic agent, including but not limited to chlorite, is diminished by exposure to high local acidity. “Diminished activity,” as used herein, refers to an activity of a therapeutic agent that is qualitatively or quantitatively inferior to that of the therapeutic agent prior to the exposure to high local acidity. As one example, a changed activity that is qualitatively or quantitatively inferior to that of the therapeutic agent prior to the exposure to high local acidity would be a lesser efficacy of wound healing, or a lesser efficacy in treating one or more of the diseases or conditions described herein. In some variations, the changed activity is any of at least about 3%, at least about 5%, at least about 10%, at least about 15%, at least about 20%, or at least about 25% lower than the activity of the therapeutic agent prior to the exposure to high local acidity. In some variations, the changed activity is at least about 5% lower than the activity of the therapeutic agent prior to the exposure to high local acidity.

In some embodiments, the pH of a chlorite formulation is adjusted to any one or more of the pH levels described in the formulations section or elsewhere herein. In some embodiments, the pH of a chlorite formulation described between about 7 and about 11.5. In some embodiments, the method comprises lowering the pH of a formulation comprising chlorite to any of between about between about 7 and about 11; between about 7 and about 10.5; between about 7 and about 10; between about 7 and about 9.5; between about 7 and about 9; between about 7 and about 8.5; between about 7 and about 8.0; between about 7 and about 7.5; between about 7.5 and about 8; between about 7.5 and about 8.5; between about 7 and about 8; between about 7.1 and about 7.7; between about 7.2 and about 7.6; between about 7.3 and about 7.5; between about 8 and about 9; between about 8 and about 8.5; between about 8.5 and about 9; about 7.0; about 7.1; about 7.2; about 7.3; about 7.4; about 7.5; about 7.6; about 7.7; about 7.8; about 7.9; about 8.0; about 8.1; about 8.2; about 8.3; about 8.4; about 8.5; about 8.6; about 8.7; about 8.8; or about 8.9 using a pH adjusting agent that does not expose the chlorite to a high local acidity. In some embodiments, the method comprises lowering the pH of a formulation comprising chlorite to between about 7 and about 8.5. In some embodiments, the method comprises lowering the pH of a formulation comprising chlorite to between about 7 and about 8.0. In some embodiments, the method comprises lowering the pH of a formulation comprising chlorite to between about 7.1 and about 7.7. In some embodiments, the method comprises lowering the pH of a formulation comprising chlorite to about 7.4.

In one non-limiting example, the pH of a mixture comprising chlorite is adjusted using a pH adjusting agent that does not subject the chlorite to a local pH of below 7 when exposed to the mixture comprising chlorite. In some embodiments, the pH adjusting agent is monosodium phosphate, disodium phosphate, or a mixture thereof. In some embodiments, monosodium phosphate and/or disodium phosphate is used as a solid or in solution. In some embodiments, the pH adjusting agent is acetic acid.

In some embodiments, the pH of chlorite is adjusted by adding chlorite or an aqueous mixture comprising chlorite to a solution containing buffer. In some embodiments, the pH of chlorite is adjusted by adding chlorite or an aqueous mixture comprising chlorite to a solution of a phosphate buffer.

In some variations, one or more pH-adjusting agents are used to adjust the pH of a chlorite solution or mixture, and the resulting solution or mixture is analyzed for the presence of degradation products of chlorite, including but not limited to degradation products generated by high local acidity. In some variations, pH-adjusting agents such as acetic acid, monosodium phosphate, and/or disodium phosphate are used to adjust the pH of a chlorite solution or mixture, and the resulting solution or mixture is analyzed for the presence of chlorate or chlorine dioxide.

In some embodiments, the resulting solution or mixture is analyzed for degradation products using well known analytical methods such as HPLC, mass spectrometry, etc. In some embodiments, the resulting solution or mixture is analyzed for degradation products using a toxicity assay, including well-known toxicity assays. In some embodiments, the resulting solution or mixture is analyzed for impurities using a non-specific toxicity assay.

In some embodiments, the pH of a chlorite formulation is adjusted after a chlorite purification step. In some embodiments, the pH of a chlorite formulation is adjusted to between about 7 and about 11.5 without the generation of chlorite degradation products that are a result of high local acidity. In some embodiments, the pH of a chlorite formulation is adjusted to between about 7 and about 8.0 without the generation of chlorite degradation products that are a result of high local acidity. In some embodiments, the pH of the chlorite formulation is adjusted to any of between about 7 and about 11; between about 7 and about 10.5; between about 7 and about 10; between about 7 and about 9.5; between about 7 and about 9; between about 7 and about 8.5; between about 7 and about 8; between about 7 and about 7.5; between about 7.5 and about 8; between about 7.5 and about 8.5; between about 7 and about 8; between about 8 and about 9; between about 8 and about 8.5; or between about 8.5 and about 9 without the generation of chlorite degradation products that are a result of high local acidity.

V. Pharmaceutical Formulations

Unless the context clearly indicates otherwise, any of the formulations described herein may be used in any of the pharmaceutical formulations described herein. In a preferred embodiment, the pharmaceutical composition can comprise: (a) chlorite; and (b) a pharmaceutically acceptable excipient. The pharmaceutical composition can further comprise a pH adjusting agent. In some embodiments, the pH adjusting agent comprises monosodium phosphate and/or disodium phosphate. The pH adjusting agent can comprise a phosphate buffer. The pH of the composition can be between about 7.1 and about 7.7, e.g., 7.4. The formulations can have low levels of harmful chlorate, e.g., the weight ratio of chlorite:chlorate can be greater than 100:1.5, or substantially free of chlorate. Such formulations can be formulated to be administered intravenously.

The pharmaceutical formulations described herein can be suitable for administration to a subject. By “suitable for administration to a subject” is meant that the pharmaceutical formulation, when obtained from a newly opened bottle and administered via the desired route, causes no greater than a clinically acceptable level of deleterious side effects.

The formulations or pharmaceutical formulations described herein can further comprise a saline solution. A saline solution, as used herein, refers to a physiologically acceptable solution with a physiologically acceptable level of sodium chloride. In some embodiments, the saline solution is isotonic.

The chlorite formulations for use with the present invention can be pharmaceutically acceptable chlorite formulations comprising one or more pharmaceutically acceptable excipients. Excipients, as used herein, refer to any non-chlorite, non-water, or non-saline element of a pharmaceutical formulation. Excipients include but are not limited to carriers, adjuvants, diluents, stabilizers, wetting agents, emulsifiers, buffers, preservatives, flavorings, inactive ingredients, gel formulations, erodible and non-erodible polymers, microspheres, liposomes, etc., including combinations of the foregoing, known to skilled artisans and described further herein. In some embodiments, the percent by weight of the excipient per the total volume of the formulation or pharmaceutical formulation is no greater than any of about 10%, about 9%, about 8%, about 7%, about 6%, about 5%, about 4%, about 3%, about 2%, about 1%, about 0.5%, about 0.4%, about 0.3%, about 0.2%, about 0.1%, or about 0.05%. In some embodiments, the percent by weight of the excipient per the total volume of the formulation or pharmaceutical formulation is no greater than about 1%. In some embodiments, the percent by weight of the excipient per the total volume of the formulation or pharmaceutical formulation is no greater than about 3%.

Below is a non-limiting and non-exhaustive list of excipients that are commonly used in the pharmaceutical arts. These excipients are commonly used in various types of formulations, including those formulated for intravenous, oral, intramuscular, or parenteral administration. Given the reactivity of chlorite, it is likely that some of the excipients listed below are inappropriate for a given pharmaceutical formulation. Whether or not a particular excipient is inappropriate for a given pharmaceutical formulation may depend upon the amount of the excipient being added to the pharmaceutical formulation. Before adding one or more of any excipient, including but not limited to the excipients described herein, to a pharmaceutical formulation of chlorite, it is important to consider the reactivity of the excipient with chlorite. Some organic molecules that are commonly used as excipients react with chlorite in such a way that the excipient is changed, including but not limited to a change that results in increased toxicity of the pharmaceutical formulation prior to exposure of the excipient to chlorite. In some embodiments, the pharmaceutical formulations described herein comprise one or more pharmaceutically acceptable excipients that do not react with chlorite. Preferably, the pharmaceutical formulations described herein comprise one or more pharmaceutically acceptable excipients that do not diminish the therapeutic effect of the pharmaceutical formulation relative to prior to exposure to the excipient.

The chlorite formulations described herein can comprise one or more pharmaceutically acceptable excipients that do not generate one or more of the deleterious non-chlorite elements of other commercially available chlorite formulations. In some embodiments, the chlorite formulations described herein comprise an excipient, and are substantially free of one or more of the deleterious non-chlorite elements of other commercially available chlorite formulations. The chlorite formulations described herein can comprise an excipient, and can be substantially free of one or more of the degradation products or impurities of other commercially available chlorite formulations as described herein.

The chlorite formulation can comprise a stabilizer. Stabilizers include but are not limited to agents that will do any of (1) improve the compatibility of excipients with a container, including a glass bottle or an encapsulating materials such as gelatin, (2) improve the stability of chlorite (e.g., prevent degradation), (3) improve formulation stability, or combinations thereof. Stabilizers may be selected from, for example, fatty acids, fatty alcohols, alcohols, long chain fatty acid esters, long chain ethers, hydrophilic derivatives of fatty acids, polyvinyl pyrrolidones, polyvinyl ethers, polyvinyl alcohols, hydrocarbons, hydrophobic polymers, moisture-absorbing polymers, and combinations thereof. Amide analogues of stabilizers can also be used. The chosen stabilizer may change the hydrophobicity of the formulation (e.g., oleic acid, waxes), or improve the mixing of various components in the formulation (e.g., ethanol), control the moisture level in the formula (e.g., PVP or polyvinyl pyrrolidone), control the mobility of the phase (substances with melting points higher than room temperature such as long chain fatty acids, alcohols, esters, ethers, amides etc. or mixtures thereof; waxes), and/or improve the compatibility of the formula with encapsulating materials (e.g., oleic acid or wax). Some of these stabilizers may be used as solvents/co-solvents (e.g., ethanol). Stabilizers may be present in sufficient amount to inhibit chlorite's degradation.

The formulations described herein may contain one or more of a gelling agent or a release modifying agent.

The formulations described herein may contain one or more adjuvants appropriate for the indicated route of administration. Again, prior to the addition of any excipient to the formulations described herein, the reactivity of chlorite should be considered with respect to whether the resulting pharmaceutical formulation will be appropriate for administration via the desired route of administration. Adjuvants with which the therapeutic agent may be admixed with include but are not limited to lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol. When a solubilized formulation is required the therapeutic agent may be in a solvent including but not limited to polyethylene glycol of various molecular weights, propylene glycol of various molecular weights, carboxymethyl cellulose colloidal solutions, methanol, ethanol, DMSO, corn oil, peanut oil, cottonseed oil, sesame oil, tragacanth gum, and/or various buffers. Other adjuvants and modes of administration are well known in the pharmaceutical art and may be used in the practice of the methods and formulations described herein. The carrier or diluent may include time delay material, such as glyceryl monostearate or glyceryl distearate alone or with a wax, or other materials well known in the art. The formulations for use as described herein may also include gel formulations, erodible and non-erodible polymers, microspheres, and liposomes.

Additives and diluents normally utilized in the pharmaceutical arts can optionally be added to the pharmaceutical composition and the liquid formulation. These include thickening, granulating, dispersing, flavoring, sweetening, coloring, and stabilizing agents, including pH stabilizers, other excipients, anti-oxidants (e.g., tocopherol, BHA, BHT, TBHQ, tocopherol acetate, ascorbyl palmitate, ascorbic acid propyl gallate, and the like), preservatives (e.g., parabens), and the like. Exemplary preservatives include, but are not limited to, benzylalcohol, ethylalcohol, benzalkonium chloride, phenol, chlorobutanol, and the like. Some antioxidants provide oxygen or peroxide inhibiting agents and may be used in the formulations described herein, including but not limited to, butylated hydroxytoluene, butylhydroxyanisole, propyl gallate, ascorbic acid palmitate, a-tocopherol, and the like. Thickening agents, such as lecithin, hydroxypropylcellulose, aluminum stearate, and the like, may be used if desired, for example to improve one or more qualities of the formulation, such as the texture.

In some variations, the chlorite formulations for use with the invention are sterile. Sterilization can be by any method that is compatible with chlorite. In some embodiments, sterilization is via a method that does not generate a substantial amount of a degradation product of chlorite. In some embodiments, sterilization is via a method that does not cause a structural change in chlorite. In some embodiments, the formulations described herein are sterile pharmaceutical formulations for parenteral or intravenous administration. In some embodiments, the chlorite formulations described herein are sterile filtered, for example, through a sterile 0.22 micron filter.

The formulations or pharmaceutical formulations can be sterile-filterable. In some embodiments, the chlorite formulations described herein are formulated for administration by one or more of the routes of administration described herein. A formulation that is “formulated for administration” by a specified route of administration, as used herein, is a formulation that does not include pharmaceutical excipients that are considered inappropriate for the route of administration by those of skill in the relevant art. As one example, a formulation that is suitable for intravenous administration would not include a toothpaste excipient or carrier intended for topical administration, where the excipient or carrier is considered inappropriate for the specified route of administration by those of skill in the relevant art.

Chlorite-containing agent in any form disclosed herein can be provided in any suitable formulation, which can be selected according to the desired route of administration as disclosed herein. In one embodiment, the formulation of the drug product comprises purified sodium chlorite which may include a certain amount of water content, buffer such as sodium phosphate dibasic, and sterile water for injection (USP) as a vehicle. In one embodiment, the amount of purified sodium chlorite is about 5.6 mg/mL (including a batch factor to reflect the water content of the batch), the amount of sodium phosphate dibasic is about 0.107 mg/mL, and sterile water to bring the volume up to 1 mL. In certain embodiments, a formulation according to the invention consists essentially of purified sodium chlorite, buffer, and sterile water for injection (USP) as the vehicle. In certain embodiments, the formulated drug product is stable for up to 3 months at 25.degree. C./60% relative humidity and/or 40.degree. C./75% relative humidity conditions.

U.S. Pat. No. 4,725,437 describes an aqueous solution of a chemically stabilized chlorite matrix suitable for intravenous administration in a dosed amount of about 6.2×10⁻⁶ mole of ClO₂ to 9.3×10⁻⁵ mole of ClO₂ per kg of body weight in humans and non-human animals. The solution contains the chlorite matrix in a concentration of about 12 to 72 micromol of ClO₂ per ml. Further chlorite formulations are described in U.S. Pat. Nos. 4,507,825, and 4,725,437, which are herein incorporated by reference in their entireties.

The present invention also provides methods of treating diseases or complications comprising administering an effective amount of TCDO in a subject. Formulations of TCDO are provided in this application. In one example, the TCDO formulation is WF10. WF10 is also known as Oxoferin® and is available commercially. In another example, the chlorite formulation contains chlorite. Other formulations of TCDO or chlorite are encompassed within the scope of the present invention. Alternatively, in some embodiments TCDO and/or WF10 can be excluded in part or in whole.

Chlorite-containing compositions, such as TCDO, can be formulated for parenteral or enteral administration, generally parenteral administration. Accordingly, formulations of chlorite, or chlorite-containing agents such as TCDO and WF10, are suitable for parenteral, topical or transdermal administration, usually intravenous, intramuscular, or subcutaneous administration, and may be suitable for administration by bolus injection, sustained release (including controlled release), infusion, and the like. More details on the route of administration are disclosed herein below. In some embodiments, the administration of the chlorite containing agents is by infusion e.g., by subcutaneous or intravenous infusion, or in the form of suppositories.

VI. Administration and Dosing of Chlorite or Chlorite Containing Agents

Unless the context indicates otherwise, all of the formulations and pharmaceutical formulations described herein may be administered by any of systemic, parenteral (e.g., intramuscular, intraperitoneal, intravenous, ICV, intracisternal injection or infusion, subcutaneous injection, or implant), by inhalation spray, nebulized or aerosolized using aerosol propellants, nasal, vaginal, rectal, sublingual, urethral (e.g., urethral suppository), by infusion, intraarterial, intrathecal, intrabronchial, subcutaneous, intradermal, intravenous, intracervical, intraabdominal, intracranial, intrapulmonary, intrathoracic, intratracheal, nasal routes, oral administration that delivers the therapeutic agent systemically, drug delivery device, or by a dermal patch that delivers the therapeutic agent systemically, transdermally or transbuccally. In some variations, the formulation is formulated for other than oral or transbuccal administration.

In some variations, the formulations described herein are not administered topically.

In some embodiments, the formulations, pharmaceutical formulations, and methods of administration and treatment described herein are suitable for use in any vertebrate, such as warm- or cold-blooded animal. In some embodiments, the formulations, pharmaceutical formulations, and methods of administration and treatment described herein are suitable for use in a mammal, including in the veterinary context, including domestic pets (such as cats, dogs, rabbits, birds, horses, etc.) and agricultural animals (such as bovine, ovine, fowl, etc.). In some variations, the formulations, pharmaceutical formulations, and methods of administration and treatment described herein are suitable for use in primates, including but not limited to humans.

Chlorite formulations are generally dosed in vivo corresponding to the body weight of the subject. Due to the continuous breakdown of the active agent in the blood, the agent is normally administered at regular intervals. Those of skill in the art will readily appreciate that actual dosages and regimen will vary as a function of the agent, formulation, the severity of the symptoms, the susceptibility of the subject to treatment and/or side effects, and the like. Dosages are readily and routinely determined by those of skill in the art by a variety of means.

Exemplary doses of chlorite-containing formulations can vary between about 0.1 ml/kg of body weight to about 1.5 ml/kg of body weight, and at a concentration of about 40 to about 80 mmol ClO₂⁻ per liter, respectively. For example, the dose of chlorite-containing formulation can comprise about 0.5 ml/kg of body weight and usually about 60 mMol ClO₂ per liter, respectively. In the case of TCDO, for example, WF10 is administered intravenously to patients with diabetes or a diabetes related disease or complication at a maximum dose of approximately 0.5 ml/kg of body weight. Other suitable doses may be approximately 0.25 ml/kg of body weight.

The regimen of administration e.g. dose combined with frequency of administration will generally involve administration in an amount and at a frequency to provide a desired effect, e.g. administration of an amount effective to provide for improvement in one or more symptoms of a patient suffering from diabetes or a diabetes related disease or complication, such as a cardiovascular disease, a metabolic disease such as metabolic syndrome, and macular degeneration symptoms. For example, chlorite or a chlorite-containing agent can be administered for 2, 3, 4, 5, 6, 7, 8, 9, 10 or more consecutive days, which administration period may be reinitiated after 1, 2, 3 or more weeks following the last dose.

The regimen of administration e.g. dose combined with frequency of administration will generally involve administration in an amount and at a frequency to provide a desired effect, e.g. administration of an amount effective to provide for improvement in one or more symptoms of a patient suffering from a macrophage-related disease or complication, such as inflammation, lesion, muscle degeneration, and obesity. Some examples of macrophage-related disease can include but is not limited to Alzheimer's disease (AD), Parkinson's disease (PD), Huntington's disease (HD), Multiple sclerosis, and Amyotrophic lateral sclerosis (ALS), cancer, and chronic granulomatous disease. For example, chlorite or a chlorite-containing agent can be administered for 2, 3, 4, 5, 6, 7, 8, 9, 10 or more consecutive days, which administration period may be reinitiated after 1, 2, 3 or more weeks following the last dose.

Chlorite according to the invention can be administered on a daily basis. In some embodiments, chlorite is administered on a daily basis at a dose of about 0.2 mg/kg/day of chlorite to about 3.3 mg/kg/day of chlorite. In some embodiments, chlorite is administered on a daily basis at a dose of about 0.2 mg/kg/day of chlorite per day, about 0.4 mg/kg/day of chlorite per day, about 0.5 mg/kg/day of chlorite, about 0.6 mg/kg/day of chlorite, about 0.7 mg/kg/day of chlorite, about 0.8 mg/kg/day of chlorite, about 0.9 mg/kg/day of chlorite, about 1.0 mg/kg/day of chlorite, about 1.1 mg/kg/day of chlorite, about 1.2 mg/kg/day of chlorite, about 1.3 mg/kg/day of chlorite, about 1.4 mg/kg/day of chlorite, about 1.5 mg/kg/day of chlorite, about 1.6 mg/kg/day of chlorite, about 1.7 mg/kg/day of chlorite, about 1.8 mg/kg/day of chlorite, about 1.9 mg/kg/day of chlorite, about 2.0 mg/kg/day of chlorite, about 2.1 mg/kg/day of chlorite, about 2.2 mg/kg/day of chlorite, about 2.3 mg/kg/day of chlorite, about 2.4 mg/kg/day of chlorite, about 2.5 mg/kg/day of chlorite, about 2.6 mg/kg/day of chlorite, about 2.7 mg/kg/day of chlorite, about 2.8 mg/kg/day of chlorite, about 2.9 mg/kg/day of chlorite, about 3.0 mg/kg/day of chlorite, about 3.1 mg/kg/day of chlorite, about 3.2 mg/kg/day of chlorite, about 3.3 mg/kg/day of chlorite, about 3.4 mg/kg/day of chlorite, or about 3.5 mg/kg/day of chlorite.

In some embodiments, the pharmaceutical composition used in the methods of the invention can be further administered in a cycle. An exemplary cycle consists of: a) a first period of time wherein the pharmaceutical composition is administered at a first dose for a first number of times; and b) a second period of time wherein the pharmaceutical composition is administered at a second dose for a second number of times. In some embodiments, the first period of time is about one week, the first number of times is about five, the second period of time is about two weeks, and the second number of times is zero. In other embodiments, the first period of time is about one week, the first number of times is about three, the second period of time is about one week, and the second number of times is zero. The first dose can be about 0.4 mg/kg/day of chlorite to about 3.3 mg/kg/day of chlorite. For example, the first dose can be about 2.1 mg/kg/day of chlorite. The cycle can be performed multiple times, e.g., about 2, 3, 4, 5, 6, 7, 8, 9, 10 or 10 or more times. In some embodiments, the cycle is performed about 2-4 times.

In some embodiments, the dosing schedule consists of periods of administration alternating with periods of non-administration. For example, chlorite might be administered in a three week cycle, comprising dosing chlorite up to 5 times in a week followed by two weeks without treatment. The cycle could be repeated as necessary to achieve the desired result. In another embodiment, chlorite is administered in a two week cycle, e.g., up to 3 times in a week followed by a week without administration. In some embodiments, a total of 2-4 cycles are performed. In an exemplary embodiment, the dosing regimen comprises administration of 2.1 mg/kg/day of chlorite for a total of 2-4 three week cycles.

B. Macrophage Activation

In one aspect, the present invention provides a method of treating a subject suffering from a macrophage-related disease comprising administering an effective amount of an oxidative agent to a subject in need thereof. The macrophage-related disease can be related to activated macrophage. The subject suffering from a macrophage-related disease may have a plasma level of one or more inflammation factors that is higher than a threshold level, its normal level or its disease level. The oxidative agent may include, but is not limited to, chlorite. The term “normal level” refers to the average concentration of a factor measured in subjects that are not suffering from the macrophage-related disease to be treated by the administering of the oxidative agent. The term “disease level” refers to the average concentration of a factor measured in subjects that are suffering from the macrophage-related disease to be treated by administering of the oxidative agent.

Macrophages are released from the bone marrow as immature monocytes, circulated in the blood stream, and can eventually migrate into tissues to undergo final differentiation into resident macrophages. Resident macrophages include Kupffer cells in the liver, alveolar macrophages in the lung, and osteoclasts in the bone. Monocytes and macrophages are phagocytes, acting in innate immunity as well as to help adaptive immunity of vertebrate animals. Their role is to phagocytose (engulf and then digest) cellular debris and pathogens either as stationary or mobile cells, and to stimulate lymphocytes and other immune cells to respond to the pathogen. They can be identified by specific expression of a number of proteins including CD14, CD11b, F4/80 (mice)/EMR1 (human), Lysozyme M, MAC-1/MAC-3, and CD68 by flow cytometry or immunohistochemical staining (Khazen W, et al. 2005 FEBS Lett. 579 (25): 5631-4). When a monocyte enters damaged tissue through the endothelium of a blood vessel (a process known as the leukocyte extravasation), it undergoes a series of changes to become a macrophage. Monocytes are typically attracted to a damaged site by chemical substances through chemotaxis, triggered by a range of stimuli including damaged cells, pathogens and cytokines released by macrophages already at the site. At some sites such as the testis, macrophages have been shown to populate the organ through proliferation. Unlike short-lived neutrophils, macrophages survive longer in the body up to a maximum of several months.

Macrophages perform a multitude of functions essential for tissue remodeling, inflammation, and immunity, including but not limited to phagocytosis, cytotoxicity, and secretion of a variety of cytokines, growth factors, lysozymes, proteases, complement components, coagulation factors, and prostaglandins. One important role of the macrophage is the removal of necrotic cellular debris in the lungs. Removing dead cell material is important in chronic inflammation as the early stages of inflammation are dominated by neutrophil granulocytes, which are ingested by macrophages if they come of age. The removal of necrotic tissue is to a greater extent handled by fixed macrophages, which typically stay at strategic locations such as the lungs, liver, neural tissue, bone, spleen and connective tissue, where microbial invasion or accumulation of dust is likely to occur, ingesting foreign materials such as pathogens, recruiting additional macrophages if needed. Macrophages can express paracrine functions within organs that are specific to the function of that organ. In the testis for example, macrophages have been shown to be able to interact with Leydig cells by secreting 25-hydroxycholesterol, an oxysterol that can be converted to testosterone by neighboring Leydig cells. Also, testicular macrophages may participate in creating an immune privileged environment in the testis, and in mediating infertility during inflammation of the testis. A list of different types of macrophages in tissues is shown in Table 1.

TABLE 1
Different Types of Macrophages in Tissues

| Name of cell | Location |
|---|---|
| Dust cells/Alveolar macrophages | pulmonary alveolus of lungs |
| Histiocytes | connective tissue |
| Kupffer cells | liver |
| Microglia | neural tissue |
| Epithelioid cells | granulomas |
| Osteoclasts | bone |
| Sinusoidal lining cells | spleen |
| Mesangial cells | kidney |

Macrophages as scavengers that remove dying cells and other debris from the body. They are a type of antigen presenting cells which play a crucial role in initiating an immune response. As secretory cells, monocytes and macrophages are vital to the regulation of immune responses and the development of inflammation as they produce monokines including enzymes, complement proteins, and regulatory factors such as interleukin-1 Macrophages also carry receptors for lymphokines for lymphocyte activation important for killing microbes and tumor cells. After digesting a pathogen, a macrophage presents the antigen on a MHC class II molecule to the corresponding helper T cell. Eventually the antigen presentation results in the production of antibodies that bind to the antigens of pathogens, leading to phagocytosis or antibody-dependent cell cytotoxicity by macrophages. The antigen presentation on the surface of infected macrophages (in the context of MHC class II) in a lymph node stimulates TH1 (type 1 helper T cells) to proliferate (mainly due to IL-12 secretion from the macrophage). When a B-cell in the lymph node recognizes the same unprocessed surface antigen on the microbe with its surface bound antibody, the antigen is endocytosed and processed. The processed antigen is then presented in MHCII on the surface of the B-cell. TH1 receptor that has proliferated recognizes the antigen-MHCII complex (with co-stimulatory factors—CD40 and CD40L) and causes the B-cell to produce antibodies that help opsonisation of the antigen so that the pathogen can be better cleared by macrophages.

Macrophages provide yet another line of defense against tumor cells and somatic cells infected with fungus or parasites. Once a T cell has recognized its particular antigen on the surface of an aberrant cell, the T cell becomes an activated effector cell producing lymphokines including families of interleukins, chemokines and interferons that further stimulate and activate macrophages. These activated macrophages can then engulf and digest affected cells more efficiently. The macrophage does not generate a response specific for an antigen, but attacks the cells present in the local area in which it was activated.

Macrophages also play a role in muscle regeneration. A previous study has shown macrophage influences on muscle repair of soleus muscle on mice (Tidball J G, Wehling-Henricks M, 2007, The Journal of Physiology 578: 327-336). Macrophage depletion also reduces muscle growth during a growth period.

I. Classically Activated Macrophages

In one aspect, the present invention provides a method of modulating macrophage accumulation or activation comprising administering an effective amount of an oxidative agent (e.g., chlorite). The oxidative agent can be chlorite or WF10. The oxidative agent can modulate the stimulation of macrophages via receptors expressed by macrophages including but not limited to interferon (IFN)-gamma receptor, CD14/LPS receptor, MHC II molecule, or interleukin receptors such as IL-4 and IL-13 receptors. In some embodiments, the oxidative agent modulates the release of chemokines by macrophages. In some embodiments, the oxidative agent modulates the release of pro-inflammatory cytokines such as IL-1, IL-6, IL-18, INF-g, CRP and TNF-alpha, or anti-inflammatory cytokines such as IL-10 and TGF-beta by macrophages. In some embodiments, the oxidative or immunomodulating agent modulates the release of proteolytic enzymes by macrophages. In some embodiments, the oxidative or immunomodulating agent modulates the release of extracellular matrix (ECM) related molecules by macrophages.

A model of two major macrophage classes has developed (Gordon, S. (1999) Fundamental Immunology, 4th Ed., Paul, W. E., ed., Lippincott-Raven Publishers, Philadelphia, pp. 533-545; Stein, M. et al. (1992) J. Exp. Med. 176:287). Classically activated macrophages typically exhibit a Th1-like phenotype, promoting inflammation, extracellular matrix (ECM) destruction, and apoptosis, while alternatively activated macrophages typically display a Th2-like phenotype, promoting ECM construction, cell proliferation, and angiogenesis. Although both phenotypes are important components of both the innate and adaptive immune systems, the classically activated macrophage tends to elicit chronic inflammation and tissue injury whereas the alternatively activated macrophage tends to resolve inflammation and facilitate wound healing (See reviews: Duffield, J. S. (2003) Clin. Sci. 104:27; Gordon, S. (2003) Nat. Rev. Immunol 3:23; Ma, J. et al. (2003) Cell. Mol. Life Sci. 60:2334; Mosser, D. M. (2003) J. Leukoc. Biol. 73:209).

Typically, differentiation of classically activated macrophages requires a priming signal in the form of IFN-gamma via the IFN-gamma R (Dalton, D. K. et al. (1993) Science 259:1739; Huang, S. et al. (1993) Science 259:1742). When the primed macrophage subsequently encounters an appropriate stimulus, such as bacterial LPS, it becomes classically activated. LPS is first bound by soluble LBP and then by either soluble or membrane-bound CD14. CD14 delivers LPS to the LPS recognition complex (Janeway, C. A. & R. Medzhitov (2002) Annu Rev. Immunol 20:197), which consists of at least TLR410 and MD-2 (Nagai, Y. et al. (2002) Nat. Immunol. 3:667). Pathogens and pathogen components are subsequently taken up by phagocytosis (Honey, K. & A. Y. Rudensky (2003) Nat. Rev. Immunol. 3:472) and delivered to lysosomes where they are exposed to a variety of degradation enzymes including several cathepsin cysteine proteases. Suitable antigens are processed and loaded onto MHC class II molecules in late endocytic compartments and antigen/MHCII complexes as well as co-stimulatory B7 family members are presented to T cells (Harding, C. V. et al. (2003) Curr. Opin. Immunol 15:112).

These events are followed closely by a significant change in cellular morphology and a dramatic alteration in the secretory profile of the cell. A variety of chemokines including IL-8/CXCL8, IP-10/CXCL10, MIP-1 alpha/CCL3, MIP-1 beta/CCL4, and RANTES/CCL5, are released as chemoattractants for neutrophils, immature dendritic cells, natural killer cells, and activated T cells (Luster, A. D. (2002) Curr. Opin. Immunol. 14:129). Further, several pro-inflammatory cytokines are released including IL-1 beta/IL-1F2, IL-6, and TNF-alpha/TNFSF1A. TNF-alpha also contributes to the pro-apoptotic activity of the classically activated macrophage (Boyle, J. J. et al. (2003) Arterioscler. Thromb. Vasc. Biol. 23:1553; Duffield, J. S. et al. (2001) Am. J. Pathol. 159:1397; Song, E. et al. (2000) Cell. Imunol. 204:19). TNF-alpha is accompanied by Fas Ligand/TNFSF6 secretion and NO release as a result of iNOS upregulation (Hesse, M. et al. (2001) J. Immunol. 167:6533; Thomassen, M. J. & M. S. Kavuru (2001) Int. Immunopharmacol. 1:1479; Duffield, J. S. et al. (2000) J. Immunol 164:2110; Munder, M. et al. (1998) J. Immunol 160:5347). In addition, the classically activated macrophage releases proteolytic enzymes including MMP-1, -2, -7, -9, and -12, which degrade collagen, elastin, fibronectin, and other ECM components (Chizzolini, C. et al. (2000) J. Immunol. 164:5952; Gibbs, D. F. et al. (1999) Am. J. Respir. Cell Mol. Biol. 20:1136; Gibbs, D. F. et al. (1999) Am. J. Respir. Cell Mol. Biol. 20:1145).

Although the release of these molecules is important for host defense and direction of the adaptive immune system, when uncontrolled their release can levy significant collateral damage on the microenvironment. By eliciting massive leukocyte infiltration and flooding the surrounding tissue with inflammatory mediators, pro-apoptotic factors, and matrix degrading proteases, the classically activated macrophage is capable of dismantling tissues to the point of inflicting serious injury. Tissue destruction perpetrated by chronic inflammation has been associated with the development of tumors, type 1 autoimmune diseases, and glomerulonephritis among other pathologies (Gordon, S. (2003) Nat. Rev. Immunol. 3:23; Mosser, D. M. (2003) J. Leukoc. Biol. 73:209).

In some embodiments, the methods of the present invention comprise administering an oxidative compound, e.g., chlorite, for the treatment of a macrophage related diseases. In some embodiments, the present invention provides a method for treating a macrophage related disease with an oxidative agent by modulating at least one IFN-gamma receptor. In some embodiments, the present invention provides a method for treating a macrophage related disease with an oxidative agent by modulating LPS, modulating MHC II antigen presentation pathway, modulating release of chemokines including but not limited to IL-18, IL-6, CRP, and/or IFN-g.

II. Alternatively Activated Macrophages

Differentiation of alternatively activated macrophages does not require any priming. IL-4 and/or IL-13 can act as sufficient stimuli (Stein, M. et al. (1992) J. Exp. Med. 176:287; Doherty, T. M. et al. (1993) J. Immunol. 151:7151). The binding of these factors to their respective receptors is followed by fluid-phase pinocytosis of soluble antigen (Brombacher, F. (2000) BioEssays 22:646; Montaner, L. J. et al. (1999) J. Immunol 162:4613; Conner, S. D. & S. L. Schmid (2003) Nature 422:37). Soluble antigen is then loaded onto MHC class II molecules and antigen/MHCII complexes and co-stimulatory B7 family members are subsequently displayed to T cells (Harding, C. V. et al. (2003) Curr. Opin. Immunol 15:112).

Similar to the classically activated macrophage, the alternatively activated macrophage changes its cellular morphology and secretory pattern as a result of appropriate stimulation. Leukocytes are attracted by the macrophage via its release of chemokines including MDC/CCL22 (Andrew, D. P. et al. (1998) J. Immunol 161:5027; Imai, T. et al. (1999) Int. Immunol 11:81), PARC/CCL18 (Kodelja, V. et al. (1998) J. Immunol. 160:1411; Goerdt, S. et al. (1999) Pathobiology 67:222) and TARC/CCL17. Inflammation is counteracted by the release of factors such as IL-1ra/IL-1F3 (Mantovani, A. et al. (2001) Trends Immunol 22:328), Ym1, Ym2, RELMa (Raes, G. et al. (2002) J. Leukoc. Biol. 71:597; Loke, P. et al. (2002) BMC Immunol. 3:7), IL-10, and TGF-beta. TGF-beta also functions indirectly to promote ECM building by inducing nearby fibroblasts to produce ECM components. The alternatively activated macrophage itself secretes the ECM components, Fibronectin and bIG-H3 (Gratchev, A. et al. (2001) Scand. J. Immunol 53:386), the ECM cross-linking enzyme, Trans-glutaminase (Haroon, Z. A. et al. (1999) Lab. Invest. 79:1679), and Osteopontin, which is involved in cell adhesion to the ECM (Murry, C. E. et al. (1994) Am. J. Pathol. 145:1450).

In addition, alternatively activated macrophages upregulate the enzyme Arginase I, which is involved in proline as well as polyamine biosynthesis. Proline promotes ECM construction while polyamines are involved in cell proliferation (Hesse, M. et al. (2001) J. Immunol. 167:6533). Other factors secreted by the alternatively activated macrophage that promote cell proliferation include PDGF, IGF, and TGF-beta (Song, E. et al. (2000) Cell. Imunol. 204:19; Cao, B. et al. (2000) Chin. Med. J. 113:776). These factors, along with FGF basic, TGF-alpha, and VEGF, also participate in angiogenesis (Cao, B. et al. (2000) Chin. Med. J. 113:776; Sunderkotter, C. et al. (1991) Pharmac. Ther. 51:195).

The molecules secreted by the alternatively activated macrophage work toward resolution of inflammation and promotion of wound repair due to their anti-inflammatory, fibrotic, proliferative, and angiogenic activities. This macrophage is also especially efficient at combating parasitic infections such as Schistosomiasis. In addition to its beneficial activities, the alternatively activated macrophage has been implicated in several pathologies, the most prominent of which are allergy and asthma (Duffield, J. S. (2003) Clin. Sci. 104:27; Gordon, S. (2003) Nat. Rev. Immunol. 3:23).

The present invention also encompasses methods of modulating macrophage accumulation or activation with an oxidative agent targeting a signaling pathway including but not limited to lipopolysaccharide (LPS), toll-like receptor (TLR), prostaglandin E2 (PGE2), interferon (IFN)-a, IFN-b, IFN-g, interleukin (IL)-1, IL-4, IL-6, sIL1Ra, IL-10, IL-12, IL-12p40, IL-13, IL-18, CRP, IP10, MHC (major histocompatibility complex) Class II molecules (MHCII), TNF-a, macrophage inflammatory protein 1 alpha (MIP 1-a), IFN-g-inducing factor (IGIF), macrophage-stimulating protein (MSP), inter-cellular adhesion molecule 1 (ICAM-1), colony stimulating factor 1 (CSF-1R), L-arginine, and nitric oxide signaling pathways. The oxidative agent of the present invention may target or have an effect on any receptor, cytosolic or nuclear intermediate signaling molecule, or transcription factor involved in any one of the signaling pathways disclosed herein. Examples of important signaling molecules as part of one or more signaling pathways that can be modulated by the oxidative agent of the present invention include but are not limited to TLR2, TLR4, CAT2, ICSBP, IL1-R, Tie-2, TRIF/IRF3, IFNR-I, IFNR-II, IRF1, IRF2, Raf-1, MEK1, MEK2, ERK1, ERK2, p38, MAPKK4, MAPKK6, PKC, JAK1, JAK2, STAT1, STAT3, Elk1, JNK/SAPK, AP1, Pu1, NFkB, NFAT, iNOS, USF1, ISGF3, SP1, Bc16, ATF2, c-Jun, and COX-2. Molecules important to macrophage activation or effects that can be modulated, either directly or indirectly, by the oxidative agent of the present invention include those that belong to transcription factors, cell surface receptors, cytokines, chemokines, cytokine or chemokine receptors, growth factors, interferons, interferon receptors, and adhesion molecules. Specifically, the oxidative agent of the present invention can modulate molecules including but not limited to TLR-2, TLR-4, mkp-1, COX-2, SOCS-3, Fc.gamma.R1, IFN-a, IFN-b, IFN-g, CRP, IL-4, IL-6, IL-18, IL-1Ra, IGIF, IL-b, MHCI, MHCII IAA, MHCII IAB, MHCII IEB, IP10, IL-10, cathepsin H, lysozyme, CathB, stk, TNF-a, IL-12p35, IL-12p40, MIP-1a, ICAM-1, INOS, mig, Cat-2, CIITA, ICSBP, CathL, CSF1R, GM-CSF, IRF1, IRF-2, c-fos, VEGF, IL-8, bFGF, CSF-1, EGF, MMP-2, MMP-7, MMP-9, MMP-12, EMAPII, endothelin 2, HIF-1, HIF-2, CXCL8, TGF-b, PGE2, and/or MDF.

III. Monocytes

Monocytes are known as a type of white blood cell. Monocytes have two main functions in the immune system: (1) replenish resident macrophages and dendritic cells under normal states; and (2) in response to inflammation signals, monocytes can move quickly to sites of infection in the tissues and divide/differentiate into macrophages and dendritic cells to elicit an immune response. Monocytes are produced by the bone marrow from haematopoietic stem cell precursors called monoblasts. Monocytes circulate in the bloodstream for about one to three days and then typically move into tissues throughout the body. In the tissues monocytes mature into different types of macrophages at different anatomical locations. Monocytes which migrate from the bloodstream to other tissues will then differentiate into tissue resident macrophages or dendritic cells. Macrophages are responsible for protecting tissues from foreign substances but are also suspected to be the predominant cells involved in triggering atherosclerosis. They are cells that possess a large smooth nucleus, a large area of cytoplasm and many internal vesicles for processing foreign material.

There are two types of monocytes in human blood: a) the classical monocyte, which is characterized by high level expression of the CD14 cell surface receptor (CD14++ monocyte) and b) the non-classical, pro-inflammatory monocyte with low level expression of CD14 and with additional co-expression of the CD16 receptor (CD14+CD16+ monocyte). After stimulation with microbial products the CD14+CD16+ monocytes produce high amounts of pro-inflammatory cytokines such as tumor necrosis factor (TNF-α) and interleukin-12.

An increase or decrease in the number of CD14+CD16+ monocytes has been indicated in various diseases (Loems Ziegler-Heitbrock, Journal of Leukocyte Biology, Vol 81, 2007). These CD14+CD16+ monocytes may play a role in giving rise to macrophages that contribute to the inflammation of a disease. CD14+CD16+ monocytes are involved in many inflammatory diseases including but not limited to rheumatoid arthritis, diabetes, hemodialysis, atherosclerosis, Kawasaki disease, as well as bacterial infections and viral infections, which are disclosed in more details herein below. In some embodiments, the present invention provides a method of treating a macrophage related disease comprising administering to a subject in need thereof an effective amount of an oxidative and/or immunomodulatory agent, wherein the agent modulates or has an effect on CD14+CD16+ monocytes. Monocytes are bone marrow derived precursors of tissue macrophages that are critical effectors of wound healing, clearance of bacteria and cellular debris and induction and resolution of inflammation. Macrophages that are associated with classical inflammation are termed M1 and those cells produce factors such as TNF-α, IL-1 and other pro-inflammatory factors. Macrophages that are associated with reversal of inflammation and suppression of immune responses are termed M2. In the context of ALS pathogenesis, the M2 macrophage phenotype within the spinal cord is associated with normal function, whereas the appearance of new M1 type macrophages within the spinal cord is associated with disease progression (Henkel et al., (2009) J Neuroimmune Pharmacol 4(4): 389-398).

Recent studies have shown that disease progression in the G93A strain of ALS mice is directly associated with migration of inflammatory monocytes into the spinal cord (Butovsky et al., (2012) The Journal of Clinical Investigation, 122(9): 3063-3087). Preliminary studies of NP001 in the G93A SOD1 congenic strain of mice showed a significant survival improvement in treated as compared to control mice (McGrath et al., (2010) 21^{st} international symposium on ALS/MND, Clinical Work in Progress 11-13). Inflammation associated disease progression might be affected in a manner similar to that seen in the ALS mouse model.

IV. Tumor-Associated Macrophages (TAM)

In some embodiments, the present invention provides a method of modulating tumor associated macrophages comprising administering an oxidative agent into a subject. Macrophages are prominent in the stromal compartment of virtually all types of malignancy. Macrophages respond to the presence of stimuli in different parts of tumors with the release of a distinct repertoire of growth factors, cytokines, chemokines, and enzymes that regulate tumor growth, angiogenesis, invasion, and/or metastasis. The distinct microenvironments where tumor-associated macrophages (TAM) act include: 1) areas of invasion where TAMs promote cancer cell motility; 2) stromal and perivascular areas where TAMs promote metastasis; and 3) avascular and perinecrotic areas where hypoxic TAMs stimulate angiogenesis (reviewed by Lewis C E et al. Cancer Res. 2006 (66) 605-612). TAMs have a phenotype that are relatively immature, characterized by low expression of the differentiation-associated macrophage antigens, carboxypeptidase M and CD51, high constitutive expression of IL-1 and IL-6, and low expression of TNF-a.

TAM infiltration correlates positively with tumor cell proliferation as measured by MIB-1 levels in breast carcinomas, Ki67 levels in endometrial carcinomas, or mitotic index in renal cell carcinoma (reviewed by Lewis C E et al. Cancer Res. 2006 (66) 605-612). Various studies have shown that TAMs express a number of factors that stimulate tumor cell proliferation and survival, including epidermal growth factor (EGF) (Goswami S. et al. Cancer Res 2005; 65; 5278-83; Lewis C E et al. Lancet 1993; 342; 148-9), platelet-derived growth factor (PDGF), TGF-h1, hepatocyte growth factor, MMP-9, and basic fibroblast growth factor (bFGF). TAMs also play an important part in regulating angiogenesis. TAMs release a number of potent proangiogenic cytokines and growth factors, such as vascular endothelial growth factor (VEGF), TNF-a, IL-8, and bFGF. Additionally, they express a broad array of angiogenesis-modulating enzymes, including MMP-2, MMP-7, MMP-9, MMP-12, and cyclooxygenase-2 (COX-2) (Sunderkotter C. et al. Pharmacol Ther 1991; 51: 195-216; Klimp A H et al. Cancer Res. 2001; 61: 7305-9). TAMs respond to tumor hypoxia by upregulating the hypoxia-inducible transcription factors HIF-1 and HIF-2. Macrophages also upregulate VEGF and other proangiogenic factors in response to hypoxia. For example, macrophages synthesize elevated levels of MMP-7 when exposed to hypoxia in vitro and in avascular areas of human tumors. A cDNA array study has identified upregulation of messages encoding more than 30 other proangiogenic genes in primary macrophages exposed to hypoxia, including CXCL8, angiopoietin, COX-2 and other factors (White J R, et al. Genomics 2004; 83: 1-8).

TAMs have also been implicated in the regulation of metastasis. High numbers of TAMs in primary tumors have been correlated with early establishment of metastases in a number of tumor types (Hanada T et al. Int J. Urol 2000; 7: 263-9). TAMs play roles in both the release of metastatic cells from the primary tumor as well as the establishment of secondary tumors at distant sites.

TAMs also play a role in tumor immunosuppression. Unlike macrophages from healthy tissues, which are capable of presenting tumor-associated antigens, lysing tumor cells, and stimulating the antitumor functions of T cells and NK cells, TAMs in the tumor microenvironment lack these activities, leaving the host without the ability to mount an effective antitumor immune response. A number of studies have shown that tumor-derived molecules, like cytokines, growth factors, chemotactic molecules, and proteases, influence TAM functions (Elgert K D et al. J Leukoc Biol 1998; 64: 275-90). For example, tumor cells secrete proteins that can inhibit the cytotoxic activity of TAMs, e.g., IL-4, IL-6, IL-10, MDF, TGF-h1 and PGE 2 (Ben-Baruch, Semin Cancer Biol 2005). Moreover, TGF-h1, IL-10, and PGE 2 may suppress the expression of MHC class II molecules by macrophages in the tumor microenvironment as well as distant sites like the spleen and peritoneum. This effect may limit the ability of TAMs to present tumor-associated antigens to T cells effectively in these areas. Another important aspect of TAM involvement in antitumor immune mechanisms is the ability of these cells to release immunostimulatory cytokines. For example, macrophage expression of IL-12, a cytokine known to stimulate both the proliferation and cytotoxicity of T cells and NK cells, is markedly suppressed in tumors, possibly by exposure to IL-10, PGE 2, and TGF-h1 (Mitsuhashi M. et al. J Leuko Biol 2004; 76: 322-23). Hypoxia in the tumor microenvironment is likely to contribute suppressing the antitumor activity of TAMs as it stimulates the release of the potent immunosuppressive factors PGE 2 and IL-10. They act on TAMs to reduce their cytotoxicity activity toward tumor cells. Hypoxia also inhibits the ability of macrophages to phagocytose dead or dying cells and present antigens to T cells. One mechanism by which this may be achieved is by reduced surface expression of CD80, a costimulatory molecule needed for the full activation of T-cell responses to antigenic peptides.

Many signaling pathways are important to TAM functions. Exemplary signaling pathways regulating TAM function include but are not limited to NFkB pathway, TLR pathways, specifically TLR/IL-1R signaling, TLR2 and TLR4 signaling, the Tie-2/Ang-2 pathway, the TRIF/TBK1/IRF3 pathway, and hypoxia-induced pathways. NFkB is one of the most crucial transcription factors regulating the inflammatory repertoire of macrophages, particularly their expression of proinflammatory cytokines, costimulatory molecules, and other activation markers in response to diverse environmental cues (e.g., stress signals, inflammatory cytokines, pathogens, and hypoxia). TLR/IL-1R signaling is an important upstream component of NFkB activation in macrophages. In inflammation-induced cancers, activation of TLR/IL-1R on stromal macrophages may be triggered by: 1) direct interaction with bacteria at sites of chronic infection (e.g., enteric bacteria in colitis-associated colon cancer or H. pylori in gastric cancer) (Karin M et al. Cell 2006 124: 823-835); or 2) interaction with tumor-cell-derived proinflammatory cytokines like IL-1; and/or 3) recognition of components of necrotic tumor cell debris like HMGB1 (high mobility group box 1) or S100 (reviewed by Biswas S K et al. J. Immunol. 2008 180: 2011-2017). TLR4 activation on human lung cancer cells promotes production of the immunosuppressive cytokine TGF-.beta. and the proangiogenic factors VEGF and CXCL8 as well as conferring resistance to TNF-.alpha.-induced apoptosis and tumor cell survival (He W et al. Mol. Immunol 2007 44: 2850-2859). A preferential role of TLR2 activation in triggering an M2 (immunosuppressive)-like cytokine profile (IL-12 low, IL-10 high) in dendritic cells and macrophages through ERK/MAPK phosphorylation has been reported (Dillon S et al. J Immunol 2004 172: 4733-4743).

Tie-2-expressing monocytes (TEM) exist in human and murine tumors (De Palma et al 2005 Cancer Cell 8: 211-226). Endothelial cells as well as tumor cells are known to up-regulate Ang-2, a ligand for Tie-2 in tumors. It has been suggested that tumor-derived Ang-2 may facilitate the recruitment of Tie-2 monocytes/macrophages into tumors (Murdoch C et al. J Immunol 178: 7405-7411). Importantly, Ang-2 also significantly inhibits the release of proinflammatory cytokines like TNF-.alpha. and IL-12 by Tie-2 monocytes in vitro (Biswas S K et al. J. Immunol. 2008 180: 2011-2017), an effect more pronounced in hypoxia. These findings suggest that the Ang-2/Tie-2 axis may represent another potential mechanism for dampening the antiangiogenic phenotype and prompting the immunosuppressive phenotype of TAM, especially in hypoxic areas of tumors.

Preferential activation of the TRIF-dependent IRF3/STAT1 pathway (where TRIF is TLR/IL-1R domain-containing adaptor inducing IFN-.beta., TBK is TANK-binding kinase, and IRF is IFN regulatory factor) has been demonstrated in TAM in murine fibrosarcoma (Biswas S et al. Blood 107: 2112-2122). This was evident from the constitutive activation of STAT1 and the up-regulation of type I IFN-inducible genes including CCL5, CXCL9, and CXCL10 in the TAM under basal and LPS-activated conditions (Biswas S K et al. J. Immunol 2008 180: 2011-2017). IL-10 transcription has also been shown to be regulated by the TRIF/IRF3 pathway via TRAF3 and type I IFNs (Chang E Y et al. J Immunol 178: 6705-6709). Taken together, TRIF pathway members such as TBK1 and IRF3 may play a role in mediating the effects of TAM and may represent a potential therapeutic target.

As mentioned hereinabove, hypoxia has profound effects on macrophage functions including their migration into tumors and patterns of gene expression, especially those encoding proangiogenic cytokines and enzymes. Hypoxia induces gene expression in these cells through up-regulation of the transcription factors hypoxia-inducible factors (HIF) 1 and 2 (HIF-1 and HIF-2). Macrophages up-regulate both HIFs and subsequently a wide array of HIF target genes in hypoxic/necrotic areas of human tumors (Murdoch C et al. 2005 Int J Cancer, 117: 701-708). Most importantly, hypoxia is a potent inducer of both VEGF and MMP7 in TAM, both of which are known to support tumor angiogenesis, invasion, and metastasis. In addition, hypoxia up-regulates the expression of M2 macrophage markers like IL-10, arginase, and PGE 2. It also modulates expression of proinflammatory genes like TNF-a, IL-1, migration inhibitory factor (MIF), CCL3, and COX2.

In some embodiments, the present invention provides a method of treating cancer comprising administering an oxidative agent. In some embodiments, macrophage activation or function is modulated by the oxidative or immunomodulatory agent of the present invention such that the antitumor activity is enhanced. In some embodiments, the oxidative agent of the present invention modulates one or more pathways involved in macrophage activation or function, wherein the pathways include but are not limited to the NFkB pathway, TLR pathway, Tie-2/Ang-2 pathway, TRIF/TBK1/IRF3 pathway, hypoxia-induced pathway and any pathway involving any molecule disclosed herein.

C. Patient Selection and Monitoring of Treatment in Macrophage-Relate Diseases

In one aspect, the present invention provides a method of treating a subject suffering from a macrophage-related disease comprising administering an effective amount of an oxidative agent to a subject in need thereof. The present invention also provides identifying a sub-population of subjects that are suffering from the macrophage-related disease. The sub-population of the subjects may respond to the administration of oxidative agent more effectively than the other sub-population of the subjects suffering from the macrophage-related disease. The present invention also provides method of identifying a subject suffering the macrophage-related disease by measuring the plasma level of one or more inflammation factors. The subject suffering from a macrophage-related disease may have a plasma level of one or more inflammation factors that is higher than a threshold level, its normal level or its disease level. The oxidative agent may include, but is not limited to, chlorite.

Macrophage-related diseases can be heterogeneous. In some cases, the macrophage-related diseases can also be partially related to genetic mutations. Depending on the level of disease progression, or the cause of the disease, the effectiveness of the subject responding to the treatment of oxidative agent may vary. The present invention also provides a method of monitoring the treatment of a macrophage-related disease by administering an oxidative agent. To prevent un-neccessary treatment and better diagnosis for the sub-population of the disease, correctly identifying a subject suffering from a macrophage-related disease that can respond positively to the oxidative agent (e.g., chlorite) treatment is important.

I. Inflammation Factors Screening

In some cases, the subject suffering from a macrophage-related disease that can respond positively to the treatment of an oxidative agent administration can be identified and subsequently treated by measuring the level of one or more inflammation factors in the plasma or bloodstream of the subject. The subject can have a plasma level of the one or more inflammation factors that is higher than a threshold level, a normal level or a disease level of the one or more inflammation factors respectively. The subject that does not have a plasma level of the inflammation factors that is higher than the threshold level, the normal level or the disease level can be monitored continuously for the level of the one or more inflammation factors to determine the right timing for such treatment.

The inflammation factors can include, without limitation, IL-18, LPS, IL-6, INF-g, CRP, IL-8, wrCRP, and combinations thereof. In preferred embodiments, the subject can be identified and treated by measuring the plasma level of at least one inflammation factor, e.g., IL-18, LPS, or both. In preferred embodiments, the subject can be identified and treated by measuring the plasma level of IL-6 and INF-g.

The subject suffering from the macrophage-related disease that can respond to the treatment of oxidative agent administration positively may have a plasma level of IL-18 that is higher than a threshold level, a normal level or a disease level. The threshold level can be about or at least about or more than about 30, 40, 50, 60, 70, 80, 90, 100 pg/ml in the plasma. The threshold level can be about, at least about or more than about 60 pg/ml. The level measured can be higher than the threshold level, the normal level or the disease level by at least 10%, 15%, 20%, 25%, 30%, 35%, 40%, 45%, 50%, 60%, 70%, 80% 90%, 1 fold, 2 fold, 3 fold, 4 fold or 5 fold.

The plasma level of IL-18 can be used as a marker for screening a subject suffering from a macrophage-related disease. In some cases, comparison of the plasma levels of IL-18 prior to and after administering a composition comprising chlorite as disclosed herein can indicate the efficacy of said treatment of macrophage-related disease.

Provided herein are methods of treating a subject suffering from a macrophage-related disease, said method comprising a) selecting a subject suffering from a macrophage-related disease if said subject has an elevated plasma level of one or more inflammatory factors chosen from the group consisting of LPS, IL-6, IL-8, IL-18, IFN-g, and CRP; and b) administering to the subject a therapeutically effective amount of a pharmaceutical composition comprising chlorite.

Further provided herein are methods of diagnosing a subject suffering from a macrophage-related disease as treatable with a pharmaceutical composition comprising chlorite comprising a) measuring a plasma level of one or more inflammatory factors chosen from the group consisting of LPS, IL-6, IL-8, IL-18, IFN-g, and CRP; and b) diagnosing the subject as suffering from a macrophage-related disease treatable with the pharmaceutical composition comprising chlorite if said subject has an elevated plasma level of the one or more inflammatory factors. A subject suffering from a macrophage-related disease may be treatable with a pharmaceutical composition comprising chlorite if the plasma level of IL-18 is at least about 60 pg/mL. A subject suffering from a macrophage-related disease may be treatable with a pharmaceutical composition comprising chlorite if the plasma level of LPS is at least about 0.05 EU/mL. A subject suffering from a macrophage-related disease may be treatable with a pharmaceutical composition comprising chlorite if the plasma level of IL-6 is at least about 6 pg/mL. A subject suffering from a macrophage-related disease may be treatable with a pharmaceutical composition comprising chlorite if the plasma level of INF-g is at least about 20 pg/mL. A subject suffering from a macrophage-related disease may be treatable with a pharmaceutical composition comprising chlorite if the plasma level of CRP is at least about 1000 ng/mL.

Further provided herein are methods for selecting responders to treatment with a pharmaceutical composition comprising chlorite comprising: a) measuring a plasma level of one or more inflammatory factors chosen from the group consisting of LPS, IL-6, IL-8, IL-18, IFN-g, and CRP in a subject suffering from a macrophage-related disease; and b) selecting the subject for administration of the pharmaceutical composition of chlorite if said subject has an elevated plasma level of the one or more inflammatory factors.

The macrophage-related disease may be a neurodegenerative disease including but is not limited to Amyotrophic lateral sclerosis (ALS), Alzheimer's disease, and Parkinson's disease, or HIV-associated neurocognitive disorder (HAND). The macrophage-related disease may also be a neurodegenerative disease of infancy or childhood selected from the group consisting of the following: Achondroplasia and variants (DE), Acute cerebellar ataxia (SE), Acute delayed measles encephalitis (Lyon) (PE), Acute disseminated encephalomyelitis (LE), Acute hemorrhagic necrotizing leukoencephalitis (LE), Adrenoleukodystrophy and variants (LE), Adrenomyeloneuropathy (LE), Aicardi syndrome of flexor spasms, callosal agenesis, andoptic hypoplasia (DE), Albinism with degenerative features and variants (DE), Albright hereditary osteodystrophy (CE), Alcoholic encephalopathy (DE), Alexander fibrinoid leukodystrophy and variants (LE), Alpers poliodystrophy (PE), Alpha-aminoadipic aciduria (DE), Alpha-ketoadipic aciduria (DE), Alpha-methyl-beta-hydroxybutyric aciduria (DE), Angleman happy puppet syndrome (DE), Arginemia (DE), Arginosuccinic aciduria (DE), Aspartylglucosaminuria (DE), Ataxia telangiectasia (CE), Autism with polioencephalopathy (PE), Balo encephalitis periaxialis concentrica (LE), Bassen-Kornzweig disease (SE), Behget syndrome (CE), Behr optic-spinocerebellar degeneration (SE), Biemond posterior column ataxia (SE), Bloch-Sulzberger disease (incontinentia pigmenti) (DE), Blue diaper syndrome (DE), Canavan spongiform leukodystrophy (LE), Carbamyl phosphate synthetase deficiency (DE), Carbon monoxide encephalopathy (CE), Carnitine deficiency (DE), Carnosinemia (hypercarnosinemia) (DE), Central pontine myelinolysis (LE), Cerebrohepatorenal syndrome (Zellweger disease) (DE), Cerebrotendinous xanthomatosis (DE), Charcot-Marie-Tooth disease and variants (SE), Chediak-Higashi disease (DE), Chronic congenital “torch” encephalopathies (PE), Chronic congenital toxoplasmosis with late degeneration (PE), Chronic cytomegalovirus infection (PE), Chronic encephalopathy with liver insufficiency (CE), Chronic encephalopathy with pulmonary insufficiency (DE), Chronic hereditary spinocerebellar degeneration (SE), Chronic lymphocytic meningitis (DE), Chronic manganese encephalopathy (CE), Chronic “torch” encephalopathy with myoclonia (CE), Chronic toxic encephalopathies (PE), Citrullinemia (DE), Cockayne syndrome (LE), Cogan syndrome of interstitial keratitis, vertigo, and deafness (SE), Collagen-vascular syndromes with encephalopathy (DE), Congenital demyelinating encephalopathy (Mackay) (DE), Congenital indifference to pain (CE), Congenital myophosphorylase deficiency (SE), Conradi chondrodystrophia calcificans congenita (DE), Craniosynostoses (DE), Crigler-Najjar kernicterus and variants (CE), Cutaneous meningeal melanosis (DE), Cystathioninuria (DE), Cystinosis (DE), Cystinuria (DE), Cytosol tyrosine aminotransferase deficiency (DE), Delange-Brachmann syndrome (LE), Delange congenital muscle hypertrophy and extrapyramidal disturbances (CE), Devic neuromyelitis optica (LE), Diabetes mellitus encephalopathy (DE), Disseminated encephalomalacia with cavity formation (Stevenson, Ford) (LE), Disseminated sarcoid leukoencephalopathy (LE), Double athetosis of Vogt (status demyelinasatus) (CE), Down syndrome with dementia (DE), Dystonia musculorum deformans and variants (CE), Fabry angiokeratoma corporis diffusum (DE), Fahr disease (CE), Familial calcifying polioencephalopathy (Geylin, Penfield) (PE), Familial deteriorating extrapyramidal syndrome (CE), Familial hypertrophic interstitial neuritis (Dejerine-Sottas) (SE), Familial hypertrophic paraprotein polyneuritis (Gibberd, Gabrilescu) (SE), Familial methemoglobinemia (DE), Familial multilocular encephalomalacia (Crome, Williams) (LE), Familial olivopontocerebellar degeneration and variants (Konigsmark, Weiner) (SE), Familial paroxysmal chorea-athetosis-dystonia (CE), Familial protein intolerance (DE), Familial striatal degeneration (CE), Familial Werdnig-Hoffmann progressive spinal atrophy (SE), Farber lipogranulomatosis (LE), Fazio-Londe familial amyotrophic lateral sclerosis (SE), Fibrous dysplasia of the skull with encephalopathy (DE), Focal dermal hypoplasia (Gorlin) (DE), Ford “312” basal ganglion syndromes (CE), Ford “312” spinocerebellar syndromes (SE), Friedreich ataxia (SE), Frontotemporal dementia, Frontotemporal lobar degeneration, Fructose intolerance and variants (DE), Galactosemia and variants (DE), GM, gangliosidoses and variants (PE), GM2 gangliosidoses and variants (Tay-Sachs disease) (PE), Gaucher disease and variants (DE), Genetic cretinism (DE), Giant axonal neuropathy (SE), Glutamate dehydrogenase deficiency (spinocerebellar degeneration) (SE), Glutamyl cysteine synthetase deficiency (DE), Glutaric aciduria and variants (DE), Glutathionemia (DE), Glycerol kinase deficiency (Guggenheim) (DE), Glycopeptidosis (DE), Haas sex-linked disease with copper metabolism defect (CE), Hallervorden-Spatz disease (CE), Harada syndrome of choroiditis, vitiligo, and deafness (SE), Hartnup disease (SE), Heller dementia (PE), Hematosidosis (anabolic GM3 gangliosidosis) (PE), Hemoglobinopathy encephalopathy (DE), Hemophilic encephalopathy and variants (DE), Hereditary bulbar atrophy (Fazio-Londe) (SE), Hereditary cerebellar ataxia (Menzel, Holmes) (SE), Hereditary cerebellar ataxia with mental deficiency (Norman, Jervis) (SE), Hereditary hemorrhagic telangiectasia (DE), Hereditary macular dystrophies with encephalopathy (DE), Hereditary motor-sensory neuropathy (England, Denny-Brown) (SE), Hereditary myoclonic encephalopathy (CE), Hereditary poliodystrophy (PE), Hereditary sensory neuropathy (Hicks, Denny-Brown) (SE), Hereditary spastic paraplegia (SE), Heredofamilial brachial plexus neuritis (Taylor) (SE), Herpes zoster with myelopathy (SE), Hippel-Lindau hemangioblastosis (DE), Histidinemia and variants (DE), Histiocytosis and variants (DE), Holmes-Logan infantile CNS degeneration (CE), Holocarboxylase deficiency (Biotin) (DE), Homocarnosinuria (DE), Homocystinuria and variants (DE), Huntington disease (CE), Hunt juvenile paralysis agitans (familial) (CE), Hunt juvenile paralysis agitans (sporadic) (CE), Hyperammonemias with diffuse encephalopathy (DE), Hyper-B-alanemia (DE), Hyperendorphin syndrome of necrotizing encephalopathy (Brandt) (CE), Hyperglycinemia (nonketotic) (DE), Hyperglycinemia with valproate therapy (DE), Hyperlysinemia (DE), Hypermethionemia (DE), Hyperphenylalanemia and variants (DE), Hyperpipecolatemia (DE), Hyperprolinemia and variants (DE), Hypertryptophanemia (DE), Hypervalinemia (DE), Hypophosphatasia (DE), Hypoxic degenerative encephalopathy with infantile spasms (DE), Hypoxic degenerative polioencephalopathy (CE), Hypoxic degenerative polioencephalopathy with infantile spasms (PE), Idiopathic degenerative encephalopathy (DE), Idiopathic dementia/autism (PE), Idiopathic dementia with polioencephalopathy (PE), Idiopathic hypoparathyroidism (DE), Idiopathic sporadic polioencephalopathy (PE), Idiopathic subcortical degeneration (CE), Immunodeficiency syndromes with encephalopathy (genetic) (DE), Immunodeficiency syndromes with encephalopathy (sporadic) (DE), Infantile neuronal degeneration (Steiman, Radermacher) (CE), Infantile polymyoclonia (CE), Isovaleric acidemia (DE), Jervis cholesterol deposits with chronic encephalopathy (DE), Joseph disease, type I (SE), Juvenile Creutzfeldt-Jakob disease (CE), Juvenile disseminated sclerosis (LE), Juvenile dystonic lipidosis (CE), Juvenile neuroaxonal dystrophy (CE), Keratosis follicularis (DE), Kernicterus (CE), Krabbe globoid cell leukodystrophy and variants (LE), Kuru (CE), Lactic acidemia (DE), Lactosyl-ceramidosis (PE), Laurence-Moon-Biedl syndrome (DE), Lead encephalopathy, chronic (DE), Leber hereditary optic neuropathy (DE), Leigh subacute necrotizing encephalomyelitis and variants (CE), Lennox-Gastaut syndrome (PE), Leprechaunism (Donohue) (DE), Leprosy dementia (DE), Lesch-Nyhan disease (CE), Lethargic encephalitis of Economo (CE), Letterer-Siwe histiocytosis (DE), Leukoencephalopathy with ragged red fibers (LE), Linear sebaceous nevus of Jadassohn with encephalopathy (DE), Lipodystrophic muscular hypertrophy with encephalopathy (DE), Lowe oculocerebrorenal syndrome (PE), Lysine intolerance (DE), Malabsorption syndromes with encephalopathy (DE), Malignant papulosis (DE), Maple syrup urine disease and variants (LE), Marfan disease (DE), Marinesco-Sjogren-Garland syndrome (SE), Menkes trichopoliodystrophy (PE), Metabolic poliodystrophy (PE), Metachromatic leukodystrophy and variants (LE), Methylmalonic acidemia and variants (DE), Metrizamide encephalopathy with asterixis (CE), Mollaret recurrent meningitis (SE), Mucolipidoses and variants (PE), Mucopolysaccharidoses and variants (PE), Mucosulfatidosis (DE), Multiple cerebroretinal arteriovenous malformations (Wyborn-Mason) (CE), Multiple lipomatosis with chronic encephalopathy (DE), Multisystem neuronal degeneration (Dyck) (DE), Myoclonic encephalopathy with progressive cranial nerve palsies (Dyken) (CE), Myoclonic-plus syndromes (Dyken) (CE), Neonatal endotoxin encephalopathy (DE), Neurofibromatosis (DE), Neuroichthyosis with dementia (DE), Neuronal ceroid lipofuscinoses and variants (PE), Nevus unis lateris (DE), Niemann-Pick sphingomyelinosis and variants (PE), Norman-Wood congenital amaurotic familial idiocy (PE), Nutritional deficiency syndromes with encephalopathy (DE), Oasthouse urine disease (DE), Oligosaccharidoses and variants (PE), Ophthalmoplegia-plus syndromes (CE), Opsoclonic meningoencephalitis (CE), Opticocochlodentatic degeneration (DE), Organic mercury cerebellar degeneration (SE), Ornithine carbamylase deficiency (DE), Ornithinemia (HHH syndrome) (DE), Orthochromatic leukodystrophy and variants (LE), Osteopetrosis (DE), 5-Oxoprolinemia (glutathionine synthetase deficiency) (DE), Parry-Romberg hemifacial atrophy with encephalopathy (DE), Pelizaeus-Merzbacher disease and variants (LE), Peroxidase deficiency (Boehme) (CE), Phenylketonuria and variants (LE), Phenytoin cerebellar degeneration (SE), Phenytoin dementia/degeneration (PE), Pleonosteosis of Leri (DE), Poikiloderma congenitale (DE), Pompe disease (SE), Porphyria and variants (PE), Postpertussis encephalopathy (PE), Postvaccinal encephalopathy (PE), Primary gliosis of the brain (DE), Progeria (Hutchinson-Gilford) (DE), Progeria (Werner) (DE), Progressive dementia with photosensitivity (Kloepfer) (LE), Progressive hereditary diaphyseal dysplasia (Engelmann) (DE), Progressive hereditary nerve deafness (SE), Progressive pallidal degeneration (Winkelman) (CE), Progressive rubella panencephalitis (LE), Proprionic acidemia and variants (DE), Pyruvate carboxylase deficiency (CE), Pyruvate dehydrogenase complex deficiency (CE), Radiation-induced encephalopathy (DE), Ragged-red mitochondrial disease (Kearns-Sayre) (CE), Ramsay-Hunt dentatorubral atrophy (CE), Refsum disease (heredopathia atactica polyneuritiformis) (SE), Rendu-Osler-Weber hemangiomatosis (DE), Riley-Day dysautonomia (CE), Roussy-Levy disease (SE), Rubinstein-Taybi syndrome (DE), Saccharopinuria (DE), Salta disease (PE), Sarcosinemia (DE), Schilder encephalitis periaxialis diffusa (LE), Segawa hereditary progressive dystonia (diurnal) (CE), Seitelberger infantile neuroaxonal dystrophy (CE), Sex-linked ataxia with myoclonia and extrapyramidal signs (CE), Sex-linked leukodystrophy (LE), Sialidoses and variants (PE), Sotos cerebral gigantism (DE), Spongiform polioencephalopathies and variants (PE), Sporadic cretinism (DE), Sporadic juvenile amyotrophic lateral sclerosis (SE), Sporadic myoclonic encephalopathy (CE), Sporadic olivopontocerebellar degeneration (Dej erine-Thomas) (SE), Sporadic optic neuritis, retrobulbar neuritis (LE), Sporadic primary lateral sclerosis (SE), Sporadic progressive thalamic atrophy (CE), Sporadic spongiform encephalopathies with myoclonus (CE), Status marmoratus (CE), Sturge-Weber disease (DE), Subacute myelo-optic neuropathy (acrodermatitis enteropathica) (DE), Subacute sclerosing panencephalitis and variants (LE), Subthalamic nuclear degeneration (Malmud, Denny) (CE), Sugarman-Reed craniofacial leukoderma (DE), Sulfituria (sulfate oxidase) (DE) Supranuclear ophthalmoplegia (hereditary) (CE) Sydenham chorea (CE), Syndrome of the sea-blue histiocyte (SE), Syringomyelia (familial) (SE), Tourette syndrome (CE), Transitional diffuse sclerosis (LE), Triose phosphate isomerase deficiency (CE), Tuberous sclerosis (DE), Tyrosinemia (DE), Unverricht-Lundborg-Lafora disease (CE), Vogt-Koyanagi syndrome (SE), Waardenburg syndrome (DE), Wadia-Swami spinocerebellar degeneration (SE), Weill-Marchesani syndrome (DE), Welander-Kugelberg-Wohlfart juvenile spinal atrophy (SE), West disease (idiopathic infantile spasms with degeneration) (PE), West disease (nongenetic diffuse encephalopathy) (DE), Wilson hepatolenticular degeneration and variants (CE), Wolman encephalopathy (LE), and Xeroderma pigmentosum and variants (SE). According to the above listing, CE indicates corencephalopathies; DE indicates diffuse encephalopathies; LE indicates leukoencephalopathies; PE indicates polioencephalopathies; and SE indicates spinocerebellopathies.

IL-18, whose whole system includes IL-18, caspase-1, IL-18R and IL-18BP, is a cytokine belonging to the IL-1 family. It exerts the effect via binding to a specific receptor complex (IL-18R) and its expression can be detected in several different cell types such as monocytes, dendritic cells (DCs), Kupffer cells, keratinocytes, chondrocytes, osteoblasts and fibroblasts, despite its primary source being macrophage. Amongst brain cells, IL-18 can be mainly expressed by microglia, astrocytes, ependymal cells and neurons. Brain IL-18 expression may be enhanced in vivo during neuroinflammatory events in response to the harmful effects of diverse exogenous or endogenous insulting stimuli, like brain infection, hypoxic-ischemic, hyperoxic and traumatic brain injury. Regarding neurodegenerative diseases, especially Alzheimer's disease, signals produced by stressed, damaged or otherwise malfunctioning brain cells could activate the innate immune system through eliciting the cytokine release. Previous studies showed that components of two major families of PPRs, TLRs and NLRs are involved in Alzheimer's disease neuroinflammation and neurodegeneration.

Inappropriate TLR responses can contribute to neuroinflammation and neurodegeneration. Studies on innate immunity receptors in AD showed an interaction between aggregated Aβ and the LPS receptor CD14, which can signal by TLR4. TLR triggering, obtained by LPS treatment through CD14 binding, can result in the activation of the transcription factor NF-kB, which in turn regulates the expression of a wide array of genes involved in the activation of inflammatory responses, including IL-18. LPS can induce IL-18 expression in microglia. Another important signal for IL-18 production within a neuroinflammatory context is the activation of the inflammasome, which triggers the processing and release of the pro-inflammatory cytokines IL-1β and IL-18. Moreover, the inflammasome has the pivotal function to convert inactive procaspase-1 to active caspase-1, which is able to cleave the inactive IL-18 precursor to a secreted, active cytokine.

Without being bound by any theory, it is typically known that there are two events required for production of mature IL-18, i.e. the enhanced precursor synthesis and the precursor processing. The enhanced precursor synthesis is mainly regulated by TLR activation and transcriptionally by NF-κB. The precursor processing, on the other hand, is chiefly depends on inflammasome involvement and caspase-1 activation both of which appear to occur in Alzheimer's disease brain. Consistently, it is observed that an increased expression of IL-18 protein and caspase-1 was specifically observed in the frontal lobe of Alzheimer's disease brains. In this context, microglia, in addition to astrocytes and neurons stained with IL-18, were observed in the strict vicinity of amyloid deposits and neurofibrillary tangles. Therefore, it is highly conceivable that Alzheimer's disease-specific pathogenic insults, such as Aβ accumulation, can lead via PPRs activation to an increased release of IL-18 within the brain of Alzheimer's disease subjects.

Lipopolysaccharides (LPS), also known as lipoglycans and endotoxin, are large molecules consisting of a lipid and a polysaccharide with the polysaccharide further composed of O-antigen, outer core and inner core joined by a covalent bond. LPS is the major component of the outer membrane of Gram-negative bacteria, contributing greatly to the structural integrity of the bacteria, and protecting the membrane from certain kinds of chemical attack. LPS can also increase the negative charge of the cell membrane and helps stabilize the overall membrane structure. Moreover, LPS is an endotoxin which induces a response from normal animal immune systems.

Macrophage-related disease can be associated with inflammation or microglial activation. In general, inflammation and microglial activation is considered as a common component of the pathogenesis for multiple neurodegenerative diseases, such as Alzheimer's disease (AD), Parkinson's disease (PD), Huntington's disease (HD), Multiple sclerosis, and Amyotrophic lateral sclerosis (ALS). Microglia, the resident innate immune cells in the brain, actively monitor their environment and can become over-activated in response to diverse cues to produce cytotoxic factors, such as tumor necrosis factor alpha (TNFα). While microglial activation is necessary and critical for host defense, over-activation of microglia is neurotoxic. LPS can damage dopaminergic (DA) neurons only in the presence of microglia. LPS activation of microglia both in vivo and in vitro can cause the progressive and cumulative loss of DA neurons over time. During critical periods of embryonic development, maternal exposure to low concentrations of LPS in mice impacts microglial activation and DA neuron survival in offspring that persists into adulthood. Also, there are several reports showing that LPS activates cells in the liver to produce TNFα, which is distributed in the blood and transferred to the brain through TNFα receptors to induce the synthesis of additional TNFα and other pro-inflammatory factors, creating a persistent and self-propelling neuroinflammation that induces delayed and progressive loss of DA neurons of adult animals. LPS can convert a macrophage into an activated macrophage, and can cause unwanted inflammation.

In some cases, lipopolysaccharide (LPS) can be used as a marker for macrophage dysfunction associated with ALS. For example, circulating LPS can be an indicator of microbial translocation derived from the gastrointestinal tract and has been used to monitor progression of macrophage related diseases as shown by Brenchley et al. (Brenchley et al., Nature Med 2006). LPS was significantly increased in chronically HIV-infected individuals and in simian immunodeficiency virus (SIV)-infected rhesus macaques (Brenchley et al., (2006) Nature Med, 12: 1365-1371). Elevated level of circulating LPS can also accelerate progression of macrophage-related disease such as ALS in laboratory studies. Transgenic mice expressing a mutant form of the the superoxide dismutase 1 (SOD1) linked to ALS exacerbated disease progression by 3 weeks and motor axon degeneration after challenged intraperitoneally with a single nontoxic or repeated injection of 1 mg LPS/kg (Nguyen et al., (2004) J Neuroscience, 24(6): 1340-1349). In another study, LPS activation of macrophages in rat spinal cord was shown to cause specific loss of motor neurons (Li et al., Brain Res., (2008) 1226: 199-208). More recently, it was shown that ALS blood monocytes express LPS activation genes unrelated to disease severity (Zhang et al., JNI (2011), 230: 114-123). In our clinical trial data, early ALS with no plasma LPS progressed slower (DPR −0.55 U/month) as compared with LPS positive stage (DPR −0.88 U/month) (Neuraltus IIA trial, 2014).

The subject suffering from the macrophage-related disease that can respond to the treatment of oxidative agent administration positively may have a plasma level of LPS that is higher than a threshold level, a normal level or a disease level. The threshold level can be about or at least about or more than about 0.01, 0.05, 0.1, 0.15, 0.2 EU/ml or any detectable level in the plasma. The threshold level can be about, at least about or more than about 0.1 EU/ml. The level measured prior to treatment can be higher than the threshold level, the normal level or the disease level by at least 10%, 15%, 20%, 25%, 30%, 35%, 40%, 45%, 50%, 60%, 70%, 80%, 90%, 1 fold, 2 fold, 3 fold, 4 fold or 5 fold.

The plasma level of LPS can be used as a marker for screening a subject suffering from a macrophage-related disease. In some cases, comparison of the plasma levels of LPS prior to and after administering a composition comprising chlorite as disclosed herein can indicate the efficacy of said treatment of macrophage-related disease. The macrophage-related disease can be a neurodegenerative disease including but is not limited to Amyotrophic lateral sclerosis (ALS), Alzheimer's disease, and Parkinson's disease, or HIV-associated neurocognitive disorder (HAND). For example, the plasma levels of LPS after said treatment can be decreased by at least 10%, 15%, 20%, 25%, 30%, 35%, 40%, 45%, 50%, 60%, 70%, 80%, 90%, 1 fold, 2 fold, 3 fold, 4 fold or 5 fold, when compared to its plasma level prior to said treatment.

The subject suffering from the macrophage-related disease that can respond to the treatment of oxidative agent administration positively and may have a plasma level of IL-6 that is higher than a threshold level, a normal level or a disease level. The threshold level can be about or at least about or more than about 1, 2, 3, 4, 5, 6, 7, 8, 9, 10 pg/ml in the plasma. The threshold level can be about, at least about or more than about 6 pg/ml. The level measured prior to the treatment can be higher than the threshold level, the normal level or the disease level by at least 10%, 15%, 20%, 25%, 30%, 35%, 40%, 45%, 50%, 60%, 70%, 80%, 90%, 1 fold, 2 fold, 3 fold, 4 fold or 5 fold.

The subject suffering from the macrophage-related disease that can respond to the treatment of oxidative agent administration positively and may have a plasma level of INF-g that is higher than a threshold level, a normal level or a disease level. The threshold level can be about or at least about or more than about 5, 10, 15, 20, 25, 30, 35 or 40 pg/ml in the plasma. The threshold level can be about, at least about or more than about 20 pg/ml. The level measured prior to the treatment can be higher than the threshold level, the normal level or the disease level by at least 10%, 15%, 20%, 25%, 30%, 35%, 40%, 45%, 50%, 60%, 70%, 80%, 90%, 1 fold, 2 fold, 3 fold, 4 fold or 5 fold.

The subject suffering from the macrophage-related disease that can respond to the treatment of oxidative agent administration positively and may have a plasma level of CRP that is higher than a threshold level, a normal level or a disease level. The threshold level can be about or at least about or more than about 100, 200, 300, 400, 500, 600, 700, 800, 900, 1000, 1100, 1200, 1300, 1400, 1500, 1600, 1700, 1800, 1900, or 2000 ng/ml in the plasma. The threshold level can be about, at least about or more than about 1000 ng/ml. The level measured can be higher than the threshold level, the normal level or the disease level by at least 10%, 15%, 20%, 25%, 30%, 35%, 40%, 45%, 50%, 60%, 70%, 80%, 90%, 1 fold, 2 fold, 3 fold, 4 fold or 5 fold.

The plasma levels of IL-6 and INF-g can be used to identify, screen and subsequently treat a subject suffering from a macrophage-related disease by administering an oxidative agent to the subject. The plasma level of IL-18 can be a strong indicating marker for identifying, screening and subsequently treating a subject. The subject with the plasma level of the IL-18 that is higher than a certain level can respond to the treatment of oxidative agent administration positively. In some cases, the plasma level of LPS can be another marker for subject screening as well.

A subject that is suffering a macrophage-related disease such as ALS or AD can be admitted, and the plasma level of one or more inflammation factors can be measured. If the measured plasma level of one or more inflammation factors is found to be higher than a threshold level, a normal level or a disease level, then the subject can be subsequently treated with an oxidative agent and a positive response can be expected. If the one or more inflammation factor is found to be lower, then the subject can be advised to seek alternative treatment or continued to be monitored for the plasma level of the factors to determine an optimal timing for the treatment of the oxidative agent such as chlorite.

II. Treatment Monitoring

The present invention also provides methods for monitoring the treatment of the oxidative agent for treating a macrophage-related disease. The subject suffering from a macrophage-related disease that passes the screening by the plasma level of the one or more inflammation factors can be treated with a composition comprising an oxidative agent such as chlorite. Then the level of one or more biomarker level in the plasma can be measured in the subject during the treatment period. The measured level of biomarker can be subsequently correlated to normal and diseased levels of said biomarker and/or levels of biomarker in said subject prior to treatment. The biomarker can be selected from IL-18, LPS, IL-6, INF-g, CRP, IL-8, wrCRP and combinations thereof. In some cases, the biomarker for treatment monitoring can be IL-18. In some cases, the biomarker for treatment monitoring can be LPS.

Typically, the plasma level of biomarkers decreases after treatment of macrophage-related diseases with the composition comprising an oxidative agent disclosed herein. Non-limiting examples of biomarkers for use of monitoring the treatment can be selected from IL-18, LPS, IL-6, INF-g, CRP, IL-8, wrCRP and combinations thereof. In some cases, the plasma level of one or more biomarkers can decrease by 10%, 20%, 30%, 40%, 50%, 60%, 70%, 75%, 80%, 90%, or more. In some cases, the plasma level of one or more biomarkers can decrease to lower than about 50 pg/ml, about 40 pg/ml, about 30 pg/ml, about 20 pg/ml, about 10 pg/ml, or about 5 pg/ml, when compared to its plasma level prior to the treatment. For example, the plasma level of one or more biomarkers prior to the administration of said composition is at least about 50 pg/ml, about 40 pg/ml, about 30 pg/ml, about 20 pg/ml, about 10 pg/ml, or about 5 pg/ml. As another example, the plasma level of one or more biomarkers prior to the administration of said composition is at most about 50 pg/ml, about 40 pg/ml, about 30 pg/ml, about 20 pg/ml, about 10 pg/ml, or about 5 pg/ml. As another example, the plasma level of one or more biomarkers prior to the administration of said composition is between about 5 pg/ml to about 50 pg/ml, between about 8 pg/ml to about 12 pg/ml, between about 10 pg/ml to about 30 pg/ml, between about 15 pg/ml to about 25 pg/ml, between about 25 pg/ml to about 35 pg/ml, between about 30 pg/ml to about 45 pg/ml, or between about 40 pg/ml to about 50 pg/ml. In some cases, the plasma level can decrease to an undetectable level after administering a composition comprising an oxidative agent such as chlorite to the subject. In some cases, the plasma level of one or more biomarkers can decrease to lower than about 0.1 EU/ml, about 0.05 EU/ml, about 0.01 EU/ml, or about 0.005 EU/ml, when compared to its plasma level prior to the treatment. For example, the plasma level of one or more biomarkers prior to administration of said composition is at least about 0.1 EU/ml, about 0.05 EU/ml, about 0.01 EU/ml, or about 0.005 EU/ml. As another example, the plasma level of one or more biomarkers prior to administration of said composition is at most about 0.1 EU/ml, about 0.05 EU/ml, about 0.01 EU/ml, or about 0.005 EU/ml. As another example, the plasma level of one or more biomarkers prior to administration of said composition is between about 0.005 EU/ml to about 0.1 EU/ml, between about 0.01 to about 0.05 EU/ml, between about 0.04 to about 0.08 EU/ml, or between about 0.06 EU/ml to about 0.09 EU/ml.

The biomarker that is used for monitoring treatment can be IL-18. The level of IL-18 decreased after the administration of a composition comprising a oxidative agent such as chlorite. The treatment can be monitored by the rate of the decrease of the IL-18 plasma level. In some cases, IL-18 plasma level can decrease by 10%, 20%, 30%, 40%, 50%, 60%, 70%, 75%, 80%, 90%, or more. In some cases, the plasma level of IL-18 can decrease to lower than about 50 pg/ml, about 40 pg/ml, about 30 pg/ml, about 20 pg/ml, about 10 pg/ml, or about 5 pg/ml, when compared to its plasma level prior to the treatment.

The biomarker that is used for monitoring treatment can be LPS. The level of LPS decreased after the administration of a composition comprising a oxidative agent such as chlorite. The treatment can be monitored by the rate of the decrease of the LPS plasma level. In some cases, LPS plasma level can decrease by 10%, 20%, 30%, 40%, 50%, 60%, 70%, 75%, 80%, 90%, or more. The LPS plasma level can decrease to an undetectable level after administering a composition comprising an oxidative agent such as chlorite to the subject. In some cases, the plasma level of LPS can decrease to lower than about 0.1 EU/ml, about 0.05 EU/ml, about 0.01 EU/ml, or about 0.005 EU/ml, when compared to its plasma level prior to the treatment.

The present invention also provides methods for monitoring the inflammation progression of a macrophage-related disease by comparing the plasma level of at least one monocyte activator marker to plasma level of said monocyte activation marker in the subject prior to, and after administering said composition. The methods also provides indications for determining treatment continuation if the plasma level of said monocyte activation marker after said administering has changed compared to the plasma level of said monocyte activation marker prior to said administering. The subject suffering from a macrophage-related disease that passes the screening by the plasma level of the one or more inflammation factors can be treated with a composition comprising an oxidative agent such as chlorite. Then the level of one or more biomarker level in the plasma can be measured in the subject during the treatment period. The measured level of biomarker can be subsequently correlated to normal and diseased levels of said biomarker and/or levels of biomarker in said subject prior to treatment. The biomarker can be selected from IL-18, LPS, IL-6, INF-g, CRP, IL-8, wrCRP, CD16, HLA-DR, CD14 and combinations thereof. In some cases, the biomarker for monitoring inflammation progress can be a monocyte activation marker, e.g., CD16. In some cases, the biomarker for monitoring inflammation progress can be a monocyte activation marker, e.g., HLA-DR. In some cases, the biomarker for monitoring inflammation progress can be a monocyte activation marker, e.g., CD14. Optionally, the biomarker for monitoring inflammation progress can be a monocyte activation marker selected from HLA-DR, CD14, and CD16. The plasma level of at least one monocyte activation marker in the subject can be measured at least about 24 hours prior to, or at least 24 hours after administering the present composition. The plasma level of at least one monocyte activation marker can be elevated and higher than or at least about normal level prior to said administering. In some cases, the elevation of the plasma level of monocyte activation markers can be co-related with the rate of progression of a macrophage-related disease, e.g., elevated plasma level of monocyte activation markers can increase the rate of progress of a macrophage-related disease. Typically, the plasma level of at least one monocyte activation marker after said administering can decrease by, for example, at least about 5%, 10%, 15%, 20%, 25%, 30%, 35%, 40%, 45%, 50%, 60%, 70%, 80%, 90%, 1 fold, 2 fold, 3 fold, 4 fold, 5 fold, or more. In some cases, the plasma level of one or more monocyte activation marker can decrease to undetectable level.

Administering a composition comprising chlorite as disclosed herein can decrease the progression of a macrophage-related disease such as amyotrophic lateral sclerosis (ALS), Alzheimer's disease (AD), Parkinson's disease (PD), and HIV-associated neurocognitive disorder (HAND), other neurodegenerative disorders can include Huntington's disease (HD) and Multiple sclerosis. In some cases, using the ALSFRS-R scoring scale as an indicator, administering said composition can decrease the progression of a macrophage-related disease by at least 0.2 unit/month, 0.4 unit/month, 0.5 unit/month, 0.6 unit/month, 0.8 unit/month, 1.0 unit/month, 1.2 units/month, 1.5 units/month, 1.8 units/month, 2 units/month, 3 units/month, 4 units/month, 5 units/month, or more. For example, the progression of a macrophage-related disease can be decreased by at least 0.5 unit/month. As yet another example, the progression of a macrophage-related disease can be decreased by at least 1.0 unit/month.

### EXAMPLES

### Example 1—Treatment of ALS with Different LPS Baseline Levels

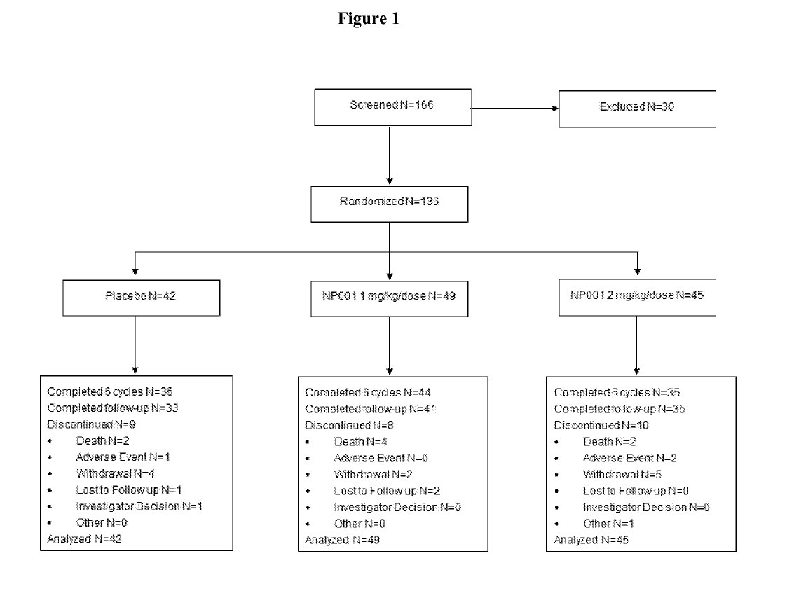

A randomized, double-blind, placebo-controlled trial of chlorite/NP001 was administered over six cycles. One hundred and thirty six men and women 21 to 80 years of age, diagnosed with possible, probable or definite ALS according to El Escorial criteria were enrolled (FIG. 1). All participants were required to have an onset of ALS-related weakness less than 3 years prior to the first dose of study medication, FVC≧70% of predicted for age and height, and a life expectancy of >6 months. Participants receiving riluzole must have been on a stable dose for >30 days. Participants on CPAP or BiPAP, those with active pulmonary disease under treatment, and those who received an immunotherapy agent within 12 weeks of randomization were excluded. Participants requiring BiPAP, CPAP, or gastrostomy after randomization could remain in the study

The study was conducted in accordance with principles of Good Clinical Practice and approved by the appropriate institutional review boards and regulatory agency for each site. Informed consent was obtained from all patients. The study was registered at clinicaltrials.gov (NCT01281631).

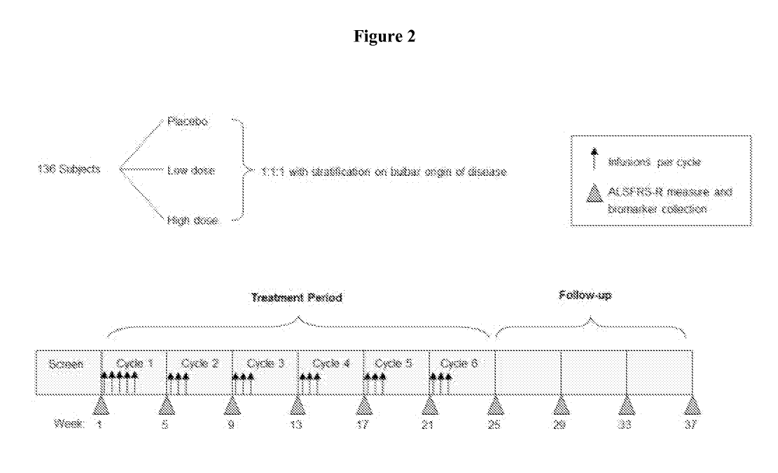

Participants were allocated in a manner 1:1:1 to receive chlorite/NP001 2 mg/kg, chlorite/NP001 1 mg/kg or placebo for a 6-month treatment period. Study drug was infused over 30 minutes by an infusion pump. Patients were scheduled to receive a total of 20 infusions over 6 cycles during a 25-week (6-month) double-blind treatment period (FIG. 2). There were approximately 4 weeks between the start of each cycle. Cycle 1/Week 1 consisted of 5 consecutive daily infusions. Cycles 2, 3, 4, 5, and 6 (Weeks 5, 9, 13, 17, and 21, respectively) each consisted of 3 consecutive daily infusions. The dosing regimen was based on prior data in an HIV population (McGrath et el., (2002) Curr. Opin. Investig. Drugs 3: 365-373). Four weeks following the final infusion (Week 25), subjects had an end-of-treatment period visit. Each patient then had a 12-week follow-period, which consisted of 3 consecutive monthly visits (Weeks 29, 33, and 37). The ALSFRS-R and VC were determined on the first day of each dosing cycle and at Weeks 25, 29, 33, and 37. Study investigators, site staff and ALSFRS-R raters remained blinded to treatment allocation throughout the study. An IDMC periodically evaluated data during the trial.

Ninety (90/136) patients completed the study. Given the exploratory nature of the study, the final sample size only had approximately 65% power to detect a 30% difference in estimated slope of decline of the ALSFRS-R (2-sided, α=0.10) over the 6-month treatment period. A secondary analytical approach, defined a priori, involved the use of ALSFRS-R data from a matched historic placebo database for the analysis of the present study. For this analysis, a sample of matched historical placebo subjects was prepared by filtering the data for the baseline characteristics (bulbar origin vs. limb onset, VC, duration of symptoms of weakness, age) from the current study and added to the placebo subject data from the present study. This allowed increased power and added precision to the point estimates, resulting in 87% power to detect a 30% improvement in disease progression as assessed by the ALSFRS-R slope.

The analysis of ALSFRS-R slope used a general linear mixed effects model with random effects to estimate the rate of decrease (slope) of ALSFRS-R, expressed as points per month, from baseline to completion of the treatment period. A secondary analysis of the slope endpoint involved the addition of matched historical placebo controls. Changes from baseline in ALSFRS-R scores using ANCOVA analyses were conducted to the end of the 25-week treatment period, from the beginning to the end of the 12-week follow-up period (Weeks 25 through 37), and from baseline to the end of the follow-up period (Weeks 1 through 37). Covariate of age, race, gender, riluzole use, duration, type and site of ALS onset, el Escorial criteria, baseline ALSFRS-R and vital capacity (VC) were utilized. Pairwise comparisons for slope and change from baseline were conducted for each dose group vs placebo group. Changes from baseline in VC for the same time periods were conducted as well as subset analyses of slope using ALSFRS-R domain subscores, gender, site of onset, and of those patients whose baseline wrCRP or monocyte chemoattractant protein-1 (MCP-1) were greater than or equal to the baseline median values for the entire enrolled population. Descriptive statistics and percent change from baseline were used to analyze the biomarker concentrations during the treatment period. Missing data were not imputed. A post-hoc exploratory analysis of the percentage of patients in each group that either improved or did not progress over the 6-month treatment period (“responders”), as assessed by change from baseline in ALSFRS-R scores, was conducted.

Safety and tolerability data were assessed by counts and tabulations of treatment-emergent adverse events (TEAEs), defined as those occurring during or after the first dose and within 30 days after the last dose of study drug, and changes from baseline in laboratory values, vital signs, physical exams and EKGs. No formal statistical analyses of TEAEs were conducted.

One hundred sixty-six patients were screened for the trial and 30 patients were excluded. A total of 136 subjects were enrolled and randomized (FIG. 1). No subjects who were randomized and received study drug and terminated early were replaced.

Approximately 95% or greater of the patients in each group completed all 5 infusions as planned in Cycle 1. The majority of subjects in each group completed 6 dosing cycles (78% to 90%). The majority of subjects in all 3 groups completed follow-up (78% to 84%).

Table 2 lists the baseline demographics and clinical features of the patient population. The groups had similar demographics and similar baseline ALS characteristics although the placebo group, numerically, had a greater percentage of patients with ALSFRS-R≧42 (28%) than the NP001 1 mg/kg (18%), and NP001 2 mg/kg (20%) group. Baseline mean wrCRP and MCP-1 values were similar between groups. There were no significant differences between groups in baseline characteristics.

TABLE 2
Baseline demographics and disease characteristics

| | Placebo | | NP001 1 mg/kg | | NP001 2 mg/kg | |
|---|---|---|---|---|---|---|
| Variable^{a} | (N = 42) | | (N = 49) | | (N = 45) | |
| Gender [n (%)] | | | | | | |
| Female | 13 | (31.0) | 13 | (26.5) | 14 | (31.1) |
| Male | 29 | (69.0) | 36 | (73.5) | 31 | (68.9) |
| Race [n (%)] | | | | | | |
| White | 41 | (97.6) | 48 | (98.0) | 43 | (95.6) |
| Black | 1 | (2.4) | 0 | (0.0) | 0 | (0.0) |
| Other | 0 | (0.0) | 0 | (0.0) | 1 | (2.2) |
| Age (years) at Enrollment | 53.7 | (9.52) | 54.4 | (12.4) | 53.6 | (10.1) |
| Duration of ALS Symptoms (mo) | 17.19 | (8.9) | 21.88 | (9.4) | 17.38 | (8.3) |
| Type of ALS [n (%)] | | | | | | |
| Familial | 5 | (11.9) | 2 | (4.1) | 2 | (4.4) |
| Sporadic | 37 | (88.1) | 47 | (95.9) | 43 | (95.6) |
| Site of ALS Onset [n (%)] | | | | | | |
| Bulbar | 7 | (16.7) | 9 | (18.4) | 8 | (17.8) |
| Limb | 35 | (83.3) | 40 | (81.6) | 37 | (82.2) |
| El Escorial Criteria for ALS [n (%)] | | | | | | |
| Probable | 19 | (45.2) | 29 | (59.2) | 23 | (51.1) |
| Definite | 21 | (50.0) | 20 | (40.8) | 20 | (44.4) |
| Concurrent Riluzole Use [n (%)] | 29 | (69.0) | 38 | (77.6) | 32 | (71.1) |
| ALSFRS-R Score at Baseline | 38.2 | (5.6) | 37.6 | (5.5) | 37.6 | (5.0) |
| Baseline MCP-1 (pg/mL) | 182.83 | (57.1) | 177.59 | (47.8) | 189.75 | (53.7) |
| Baseline wr-CRP (ng/mL) | 1941.2 | (2747.8) | 2236.2 | (2954.0) | 2992.6 | (4027.5) |
| Vital Capacity (VC) (L) at Baseline | 3.77 | (1.03) | 3.76 | (0.82) | 3.80 | (0.88) |

^{a}n = number of randomized patients. All values are means +/− SD unless otherwise indicated

Table 3 shows the most common TEAEs occurring in ≧5% of patients. No clinically relevant mean changes from baseline in vital signs or ECG parameters between treatment groups were noted.

TABLE 3
Most common TEAEs occurring in ≧5% of patients.

| | | | NP001 | | NP001 | |
|---|---|---|---|---|---|---|
| | Placebo | | 1 mg/kg | | 2 mg/kg | |
| | (N = 42) | | (N = 49) | | (N = 45) | |
| Preferred Term | n (%) | | n (%) | | n (%) | |
| Fall | 18 | (42.9) | 16 | (32.7) | 17 | (37.8) |
| Fatigue | 14 | (33.3) | 8 | (16.3) | 16 | (35.6) |
| Infusion site pain | 2 | (4.8) | 9 | (18.4) | 15 | (33.3) |
| Infusion site extravasation | 6 | (14.3) | 9 | (18.4) | 10 | (22.2) |
| Headache | 11 | (26.2) | 11 | (22.4) | 9 | (20.0) |
| Dizziness | 3 | (7.1) | 4 | (8.2) | 9 | (20.0) |
| Nausea | 6 | (14.3) | 6 | (12.2) | 7 | (15.6) |
| Cough | 4 | (9.5) | 7 | (14.3) | 7 | (15.6) |
| Infusion site erythema | 5 | (11.9) | 6 | (12.2) | 6 | (13.3) |
| Nasopharyngitis | 2 | (4.8) | 6 | (12.2) | 6 | (13.3) |
| Muscle contractions | 2 | (4.8) | 3 | (6.1) | 6 | (13.3) |
| involuntary | | | | | | |
| Back pain | 3 | (7.1) | 1 | (2.0) | 5 | (11.1) |
| Muscular weakness | 3 | (7.1) | 1 | (2.0) | 5 | (11.1) |
| Dysphagia | 5 | (11.9) | 2 | (4.1) | 4 | (8.9) |
| Constipation | 0 | (0.0) | 5 | (10.2) | 4 | (8.9) |
| Diarrhea | 1 | (2.4) | 5 | (10.2) | 4 | (8.9) |
| Rash | 0 | (0.0) | 4 | (8.2) | 4 | (8.9) |
| Contusion | 5 | (11.9) | 3 | (6.1) | 3 | (6.7) |
| Pain in extremity | 4 | (9.5) | 3 | (6.1) | 3 | (6.7) |
| Edema peripheral | 3 | (7.1) | 3 | (6.1) | 3 | (6.7) |
| Muscle spasms | 1 | (2.4) | 5 | (10.2) | 2 | (4.4) |
| Anxiety | 2 | (4.8) | 3 | (6.1) | 2 | (4.4) |
| Infusion site swelling | 3 | (7.1) | 2 | (4.1) | 2 | (4.4) |
| Nasal congestion | 3 | (7.1) | 2 | (4.1) | 2 | (4.4) |

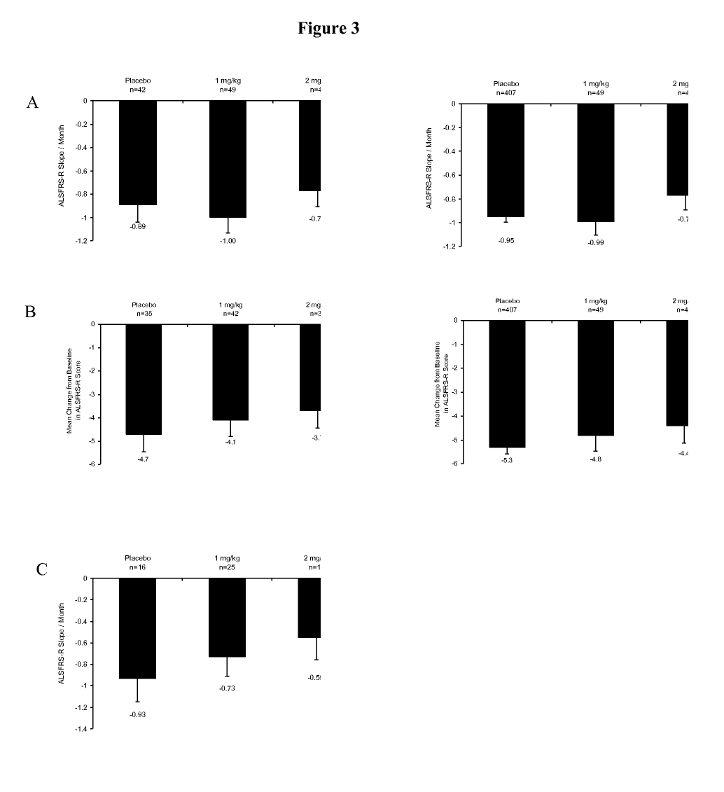

Mean slope and mean change from baseline relative to placebo in ALSFRS-R scores with and without matched historical placebo controls after 6 months of treatment (Weeks 1 through 25) are shown in FIGS. 3A and 3B.

NP001 2 mg/kg had a numerical clinical benefit compared to placebo in reducing ALS progression as shown by percent change in mean slope in points per month (13%). The mean slopes were −0.77 in the NP001 2 mg/kg group and −0.89 in the placebo group. With the addition of matched historical placebo control patients to the concurrent placebos, the mean slope for the placebo group was −0.95, thus, there was a 19% improvement in the rate of progression of the high dose group as compared to the combined placebo controls (p=0.16). Similar clinical benefits were observed for change in ALSFRS-R from baseline in the high dose group with 21% slowing (with) and 17% slowing (without) addition of matched historical placebo control patients. Similar analyses demonstrated that the NP001 1 mg/kg dose was a minimal or no effect dose compared to placebo.

FIG. 3C shows those patients treated with NP001 whose baseline wrCRP levels were at or above the median for the entire randomized population had greater slowing of progression compared to placebo patients whose baseline wrCRP values were also at or above the median. The estimated slope decline in points per month was −0.55 for the NP001 2 mg/kg group, −0.73 for the NP001 1 mg/kg group, and −0.93 for placebo. The slowing in the rate of progression in the 2 mg/kg group, represented a 41% improvement compared to placebo (p=0.2). In patients who were less than the baseline median wr-CRP, the estimated slopes were −0.87, −1.38, and −0.84 for the 2 mg/kg, 1 mg/kg, and placebo groups, respectively. The only trend for the differences in slope was for the NP001 1 mg/kg group compared to placebo (p=0.09).

During the 3-month off-drug follow-up, the mean decreases in ALSFRS-R scores were −3.3 for the NP001 2 mg/kg group, −3.7 for the NP001 1 mg/kg group, and -3.7 for placebo. Thus, during the off-drug follow-up there was slower functional decline a residual of 11% in the mean change from baseline in ALSFRS-R scores to the end of the treatment period compared to placebo in the high dose NP001 group. The clinical trends for less mean change from baseline in ALSFRS-R scores between the NP001 2 mg/kg compared to the placebo group were consistent across the different time periods (treatment and follow-up), although statistical significance was not reached.

### Example 2—Examination of Biomarkers in Responders and Non-Responders

A randomized, double-blind, placebo-controlled study was administered over six cycles. Patients were treated with chlorite 2 mg/kg/infusion, or placebo. Patients were scheduled to receive a total of 20 infusions over 6 cycles during a 25-week (or 6-month) treatment period. Patients whose change from baseline in ALSFRS-R scores after at least 6 months of treatment (Week 25) was ≧0 (i.e., ALSFRS-R scores did not decline or improved) were defined as “responders”. Samples from both responders and non-responders were collected and tested at different time points, e.g., at baseline (pre-dose Cycle 1, Week 1), 1-month, 2-month, 3-month, 4-month, 5-month and 6-month. ALSFRS-R scores and biomarkers levels were measured in responders and non-responders at selected time points and compared to the placebo group. Levels of biomarkers can be measured with techniques known in the art, e.g., ELISA or flow cytometry.

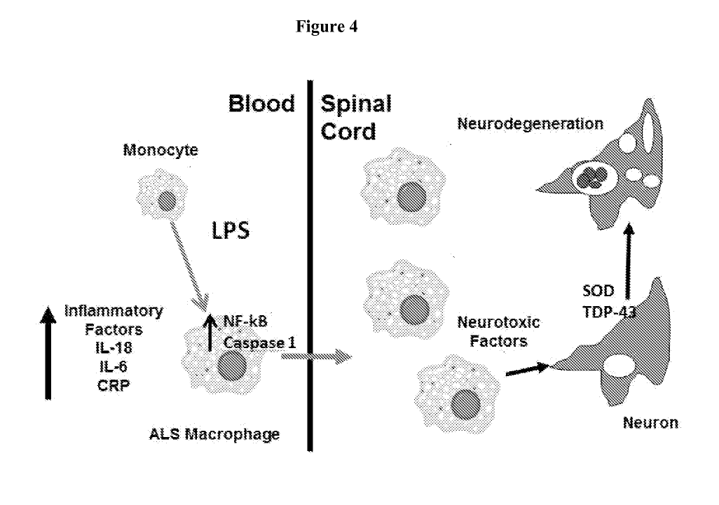

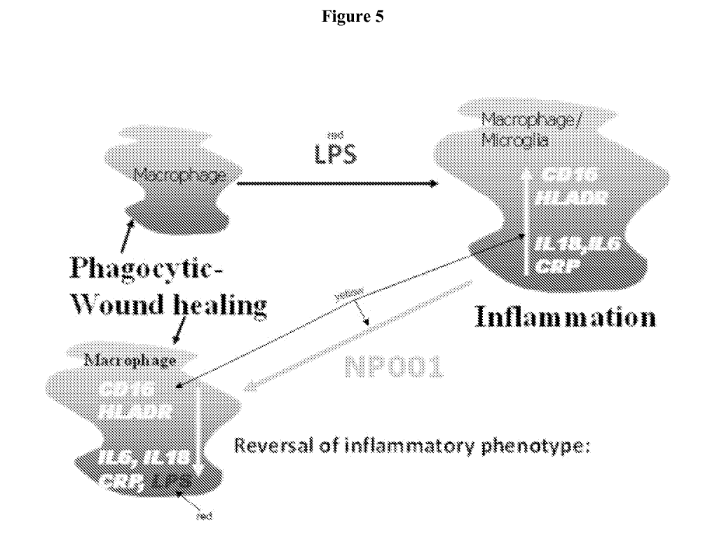

FIGS. 4 and 5 schematically illustrates the working mechanism of chlorite in treating macrophage-related diseases. In general, chlorite is converted within monocytes/macrophages into a bioactive intracellular chloramine that down-regulates NF-kB expression and inhibits production of pro-inflammatory cytokine IL-1β. With normal macrophage function, plasma LPS level would disappear and NF-kB induced inflammatory factors would be reduced.

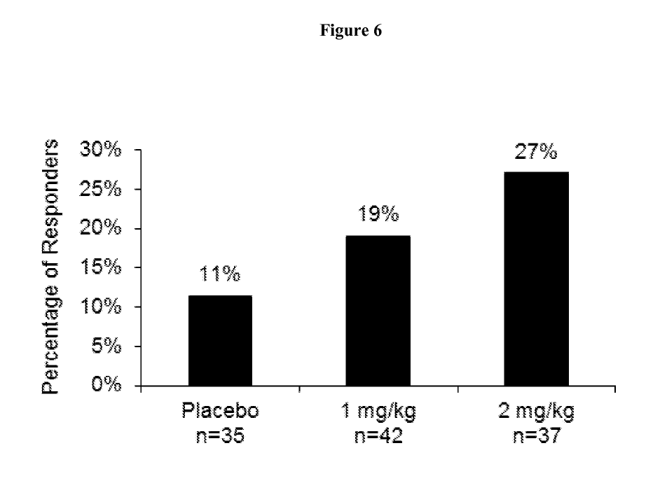

It was shown that chlorite treatment is capable of slowing the disease progression over a 6 month period of a subset of patients who received the treatment (FIG. 6). Two sub-sets of patient population were observed, denoted as responders and non-responders. A stable ALFRS-R level indicated the slowing of the progression of the disease. The ALSFRS-R score remained stable over the 6-month treatment period in responders, showing the positive responses of responders to the treatment. While the decreasing value of ALSFRS-R observed in non-responders indicated that the treatment had little or no effect on patients.

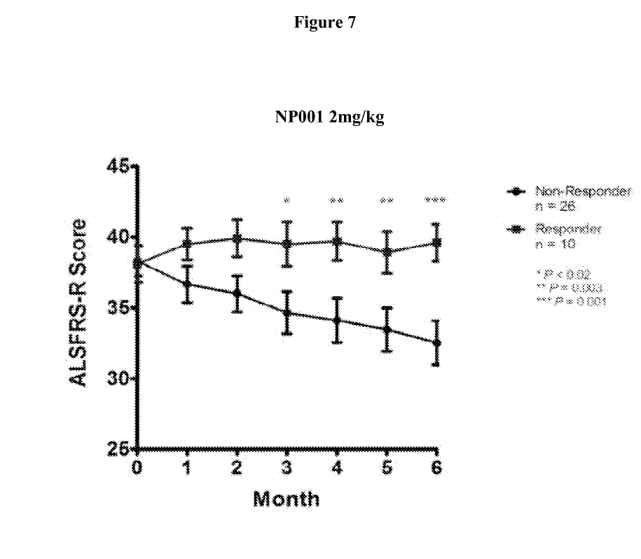

FIG. 7 shows a dose-dependent increase in the percentage of responders. In the high dose group, 27% of patients did not progress over the 6-month treatment period (FIG. 7). This is approximately 2.5 times greater than the percentage in the placebo group (11%). The superiority of the 2 mg/kg group compared to placebo became highly significant (p=0.007) with the addition of matched historical placebo control to the analysis. Consistent with these findings was a dose-dependent smaller decline, following at least 6 months of treatment, in vital capacity in responders (1 mg: −8.95+/−10.1; 2 mg: −3.76+/−5.7) compared to non-responders (1 mg: −17.7+/−17.9; 2 mg: −14.5+/−13.2).

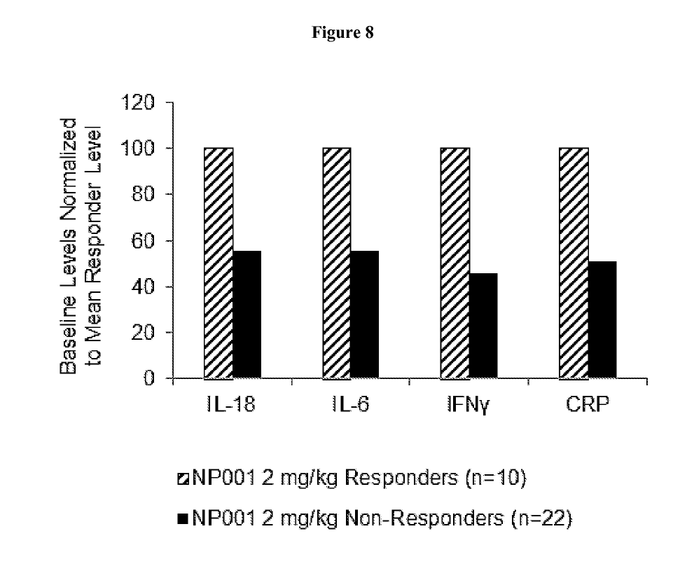

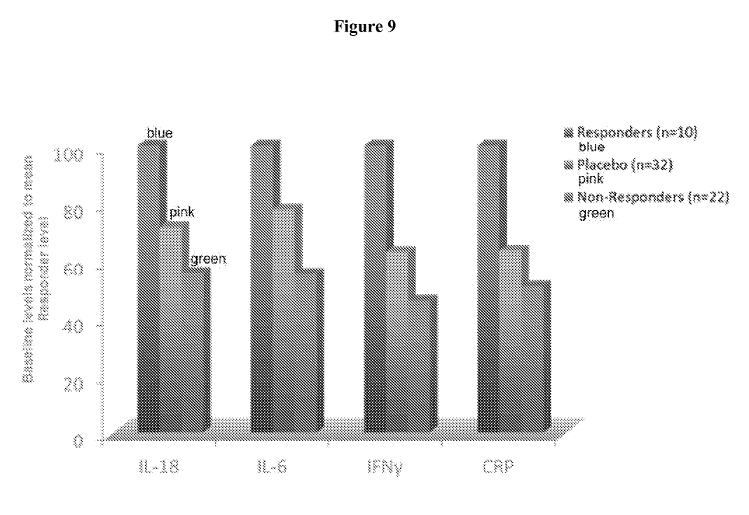

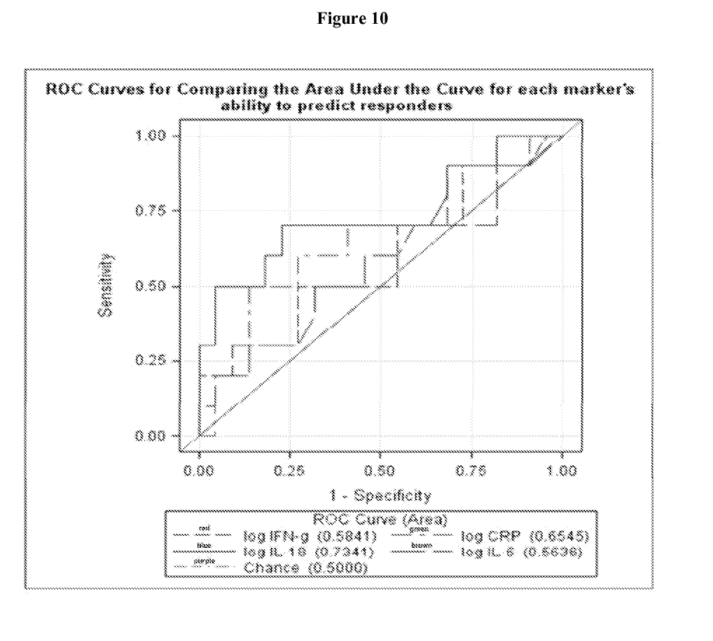

Levels of four biomarkers, i.e., IL-18, IL-6, INF-g, CRP, at baseline in responders, non-responders and placebos were also found to be different (FIG. 8). For each biomarker, its baseline levels in responders, non-responders and placebos were all normalized with respect to its baseline level in responders. Therefore, for all of the biomarkers, their baseline levels in responders were all 100. As the figure shows, responders had highest baseline levels of all the biomarkers, in comparison with non-responders and placebos. In addition, FIG. 8 and FIG. 9 show responders had elevated baseline IL-18, IL-6, IFN-gamma, and CRP compared to non-responders in the high dose group. FIG. 10 shows a ROC curve for comparing the area under the curve for each marker's ability to predict responders.

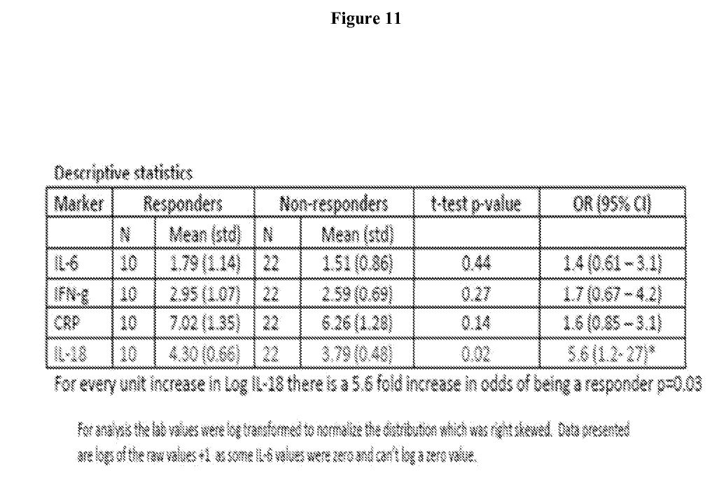

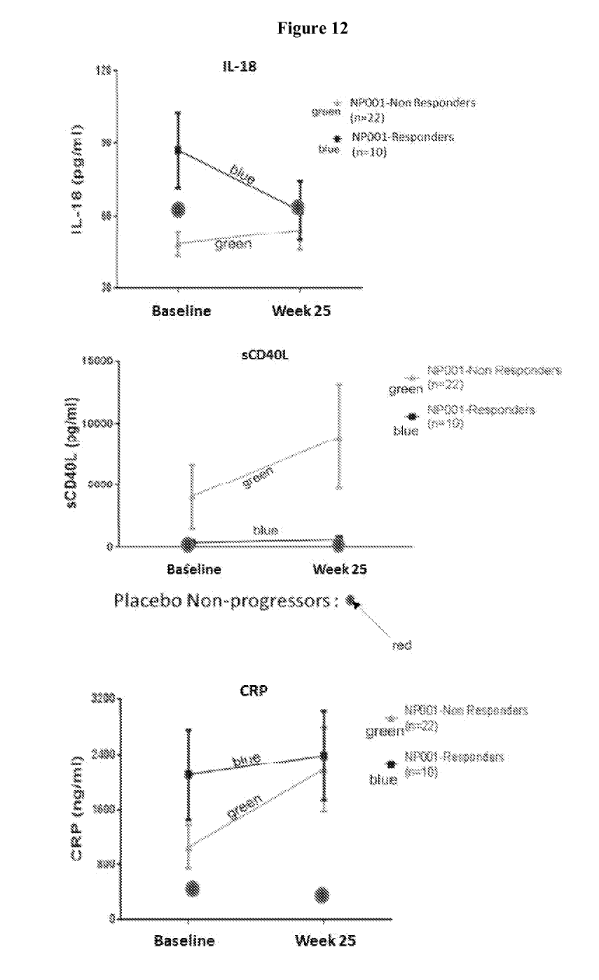

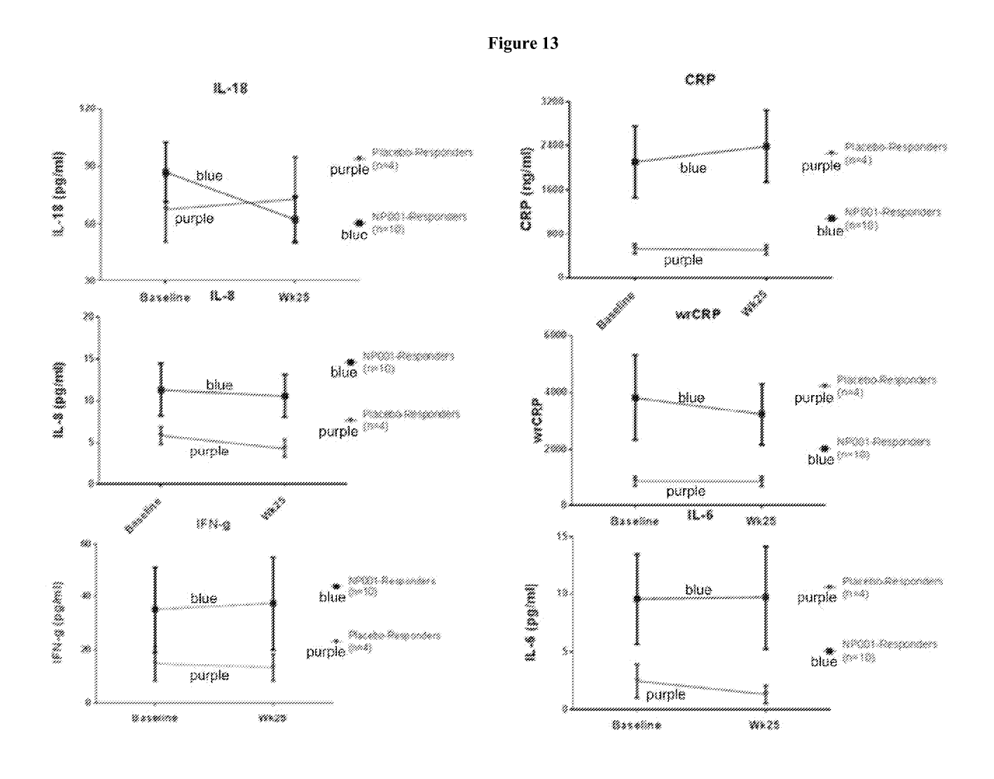

Biomarkers or inflammatory factor analysis revealed that IL-18 levels at baseline (pre-dose Cycle 1, Week 1) and Week 25 in responders, non-responders and placebos were different (FIG. 11). The plasma level of some inflammatory factors at baseline and Week 25 for responders, non-responders and placebo non-progressors was shown in FIG. 12. The “placebo non-progressors” refers to a sub-population of the placebo group that does not show disease progression in the duration of the study. FIG. 13 shows the plasma level of IL-18, IL-6, IL-8, CRP, wrCRP and INF-g at baseline and Week 25 for responders and placebo non-progressors.

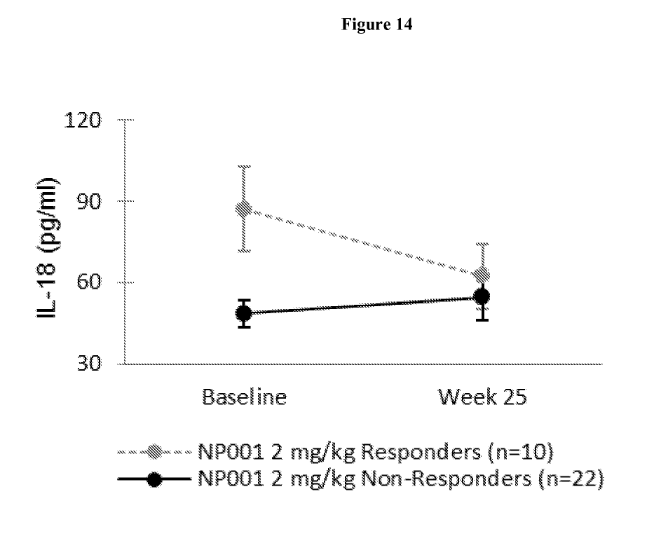

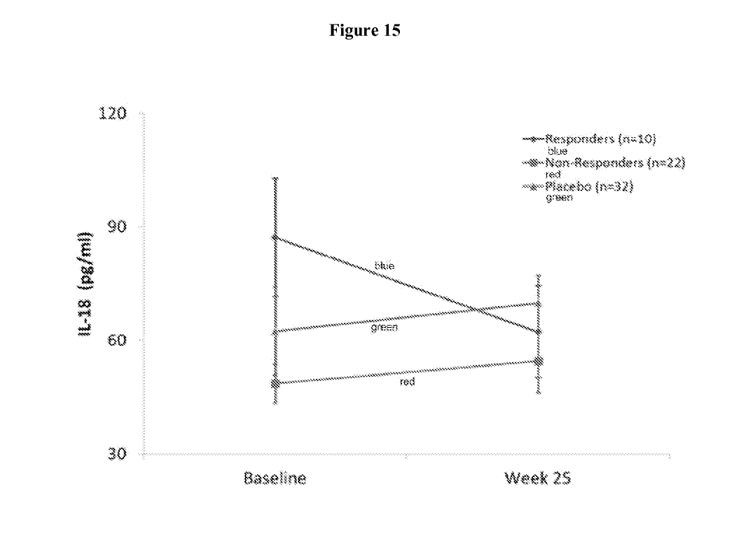

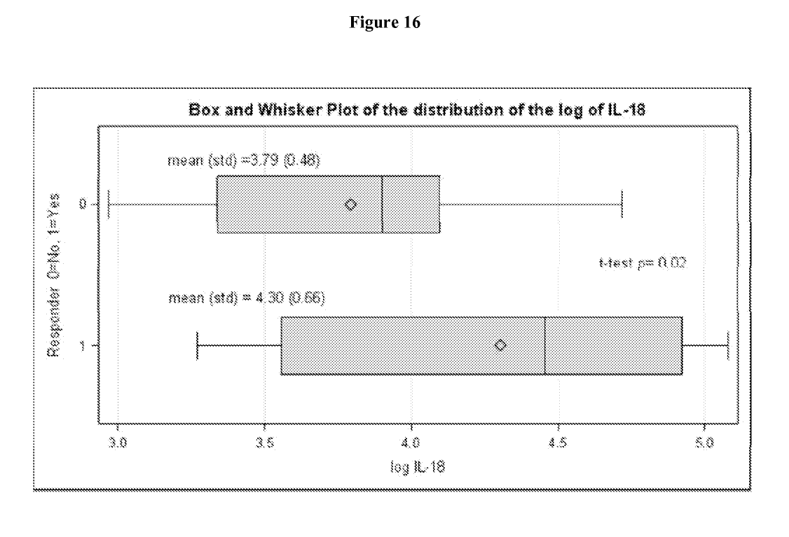

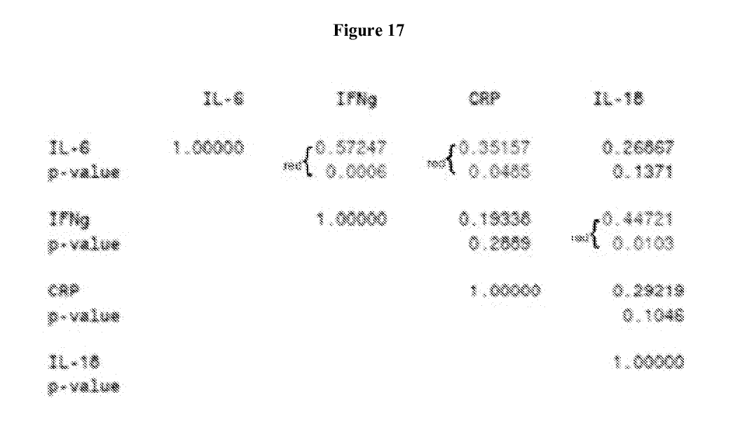

Compared with non-responders and placebos, patients who responded to the chlorite treatment by showing slowed disease progression (responders) have higher baseline level of IL-18 (FIG. 14-15). An increase in IL-18 level from the baseline occurred in both non-responders and placebos after 25 weeks, while a decrease of IL-18 level from the baseline after 25 weeks of treatment was found in responders, which showed that the treatment was effective only in responders. FIG. 16 shows a box and whisker plot of the distribution of the log of IL-18, showing that the IL-18 levels at baseline can differentiate responders and non-responders. The interrelationship of baseline values of inflammation factor plasma was shown in FIG. 17.

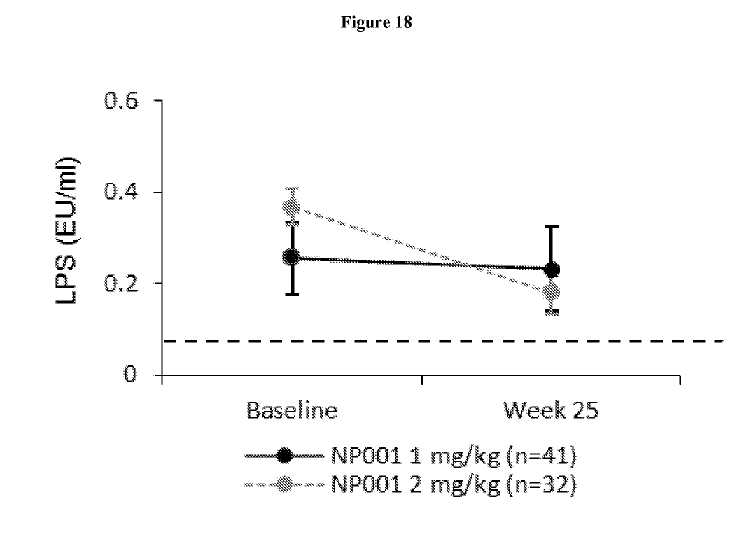

The LPS level at baseline and Week 24 in responders and non-responders was also measured. As shown in FIG. 18, with the effective treatment, the malfunction of macrophage can be cured and a decrease in LPS level can be observed. Such decrease in LPS levels was observed in both responders and non-responders after 24 weeks of treatment. For non-responders, LPS level decreased from the initial value of about 0.35 at baseline to the final value of about 0.2 at week 24, which was about 40% decrease of its baseline level. While for responders, after 24 weeks of treatment, LPS level decreased drastically from the baseline level of about 0.3 to an undetectable level at week 24. If took the minimum detection level (which was about 0.075 and still higher than the final level at week 24) for calculation, after 24 weeks of treatment, there was about 75% decrease from the baseline level which was almost 2 times higher than the decrease in non-responders. The LPS level of responders decreased to an undetectable level after 24 weeks.

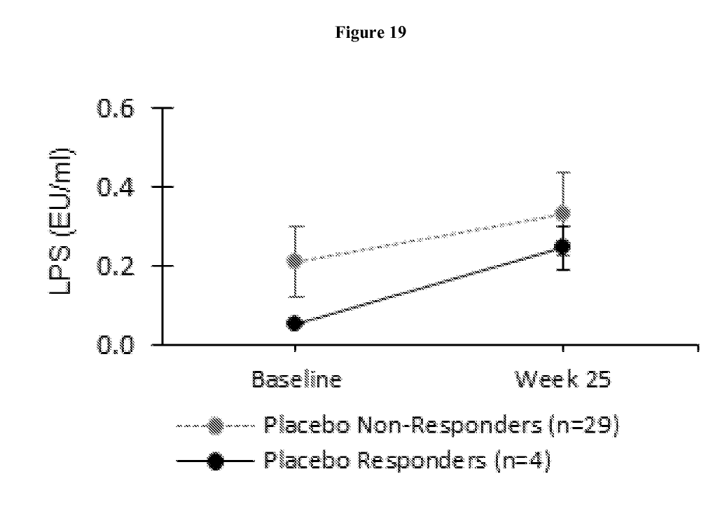

All of the high dose responders were positive for LPS in their plasma (FIG. 19). Following at least 6 months of treatment, 70% of high dose responders had decreased LPS and 80% had decreased IL-18 (FIG. 14-15, FIG. 18). Similarly in the low dose group, 7 of 8 responders were LPS positive and 75% had baseline IL-18 at or above the baseline median for all patients. Following at least 6 months of treatment, half of the patients had decreased LPS and 1 patient had decrease in IL-18. All 4 of the placebo responders were LPS negative at baseline; yet 3 of 4 had elevated IL-18. Notably LPS levels in all placebo patients (responders and non-responders) increased over the 6-month treatment period (FIG. 19).

The results of the experimentation demonstrated that chlorite/NP001 (2 mg/kg) was capable of slowing the disease progression of ALS patients with high plasma level of IL-18, IL-6, INF-g, and CPR. The LPS level of this sub-population of patients also decreased to undetectable level with chlorite treatment.

### Example 3—Examination of IL-18 Baseline Levels

Thirty two patients diagnosed with ALS were treated with chlorite over the course of 6 months. The initial plasma concentration of IL-18 in each patient was recorded and set as the baseline level. Patients were treated with 1-2 mg/kg/infusion chlorite over the course of 6 months. ALSFRS-R scores and IL-18 levels were recorded at different time points during the treatment and compared with the initial concentration.

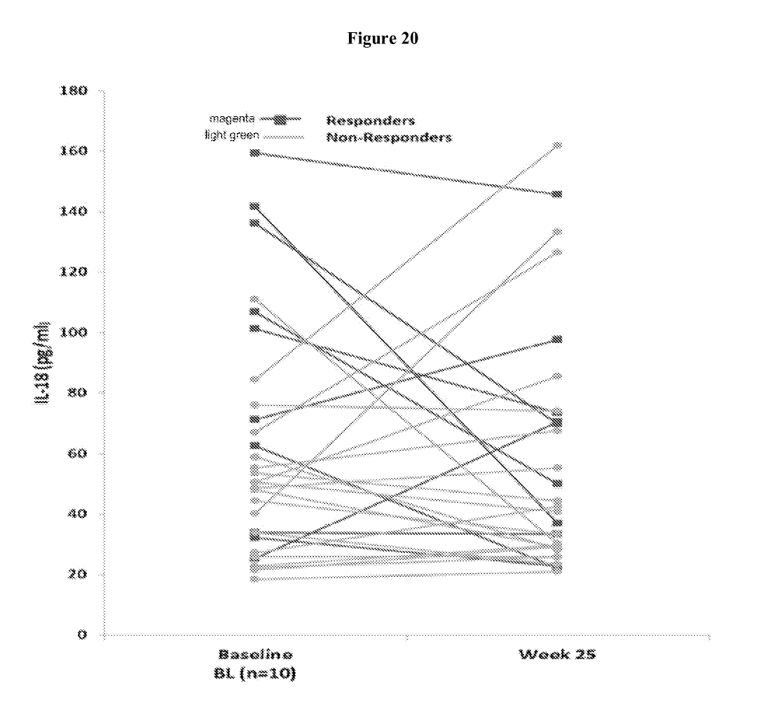

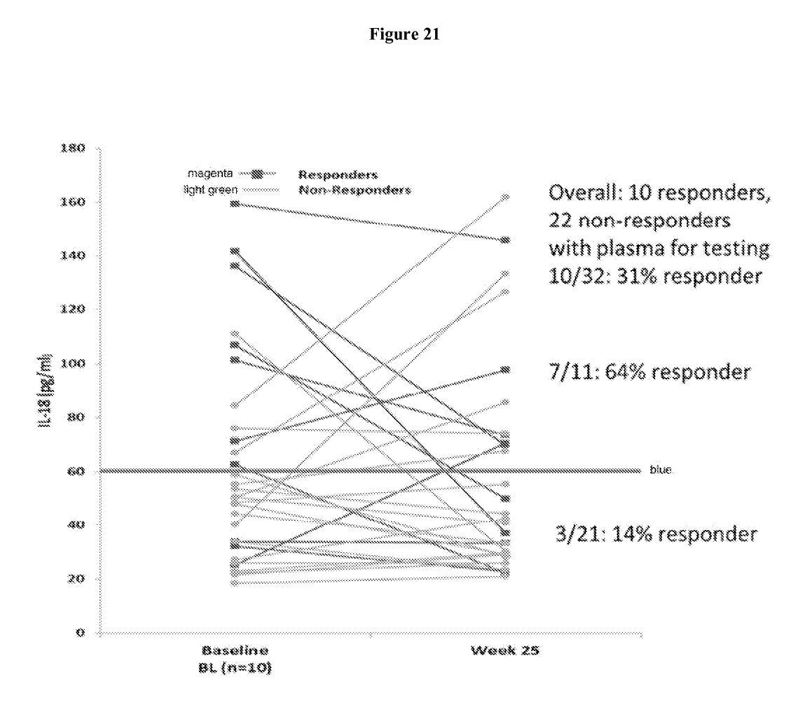

Overall, 10 responders and 22 non-responders were observed (FIG. 20-21). The plasma level of IL-18 in responders decreased after treatment with chlorite. Majority of responders had baseline IL-18 higher than 60 pg/ml (FIG. 21). By contrast, non-responder had lower baseline level of IL-18 and the majority of them had baseline IL-18 lower than 60 pg/ml.

The results of the experimentation demonstrated that chlorite/NP001 was capable of lowering IL-18 plasma level and patients with baseline IL-18 level higher than 60 pg/ml could benefit from chlorite/NP001 treatment. The results of the experimentation also suggest that baseline plasma level may be an indicator of chlorite/NP001 treatment responders.

### Example 4—Treatment of ALS Patients with Different IL-18 Baseline Levels

Patients diagnosed with ALS are treated with chlorite over the course of 6 months. Based upon the initial concentration of IL-18 at the baseline or its baseline level, patients are sorted into two groups. Patients with baseline level of IL-18 higher than 60 pg/mL are assigned in IL-18-high group and patients with baseline level of IL-18 lower than 60 pg/mL are assigned in IL-18-low group. Both groups are treated with 1-2 mg/kg/infusion chlorite over the course of 6 months. ALSFRS-R scores and IL-18 levels are recorded at different time points during the treatment and compared between IL-18-high and IL-18-low groups.

A stable ALSFRS-R value indicates the slowing of the disease progression and hence the positive response to the treatment. A decline or improvement of ALSFRS-R score shows no effect or negative response to the treatment. ALSFRS-R level stabilizes in the IL-18-high group over the whole course of treatment, showing that patients with high baseline level of IL-18 have positive response to the chlorite treatment. While the patient in the IL-18-low group, no stabilization of ALSFRS-R level can be observed, which means the treatment of chlorite has no or very little effect on the disease.

Similarly, if the chlorite is taking some effect, a decrease in IL-18 baseline level can be observed. Such decrease in IL-18 baseline level indicates the effectiveness of and the positive response to the treatment, which can only be observed in IL-18-high group.

The same results are also found out with other biomarkers which include IL-6, INF-g and CRP. In general, after treating the patients with chlorite for 6 months, a decrease in baseline levels of the biomarkers are observed only in responders whose initial baseline levels of these biomarkers are at least 20% higher than their respective cut-off levels (or disease levels).

### Example 5—Examination of Baseline LPS Level for ALS Progression

The rate of ALS progression was determined by comparing ALSFRS-R value before and after treatment with 1 mg/kg or 2 mg/kg chlorite/NP001 for 6 months. Baseline LPS level below 0.5 pg/ml was considered negative.

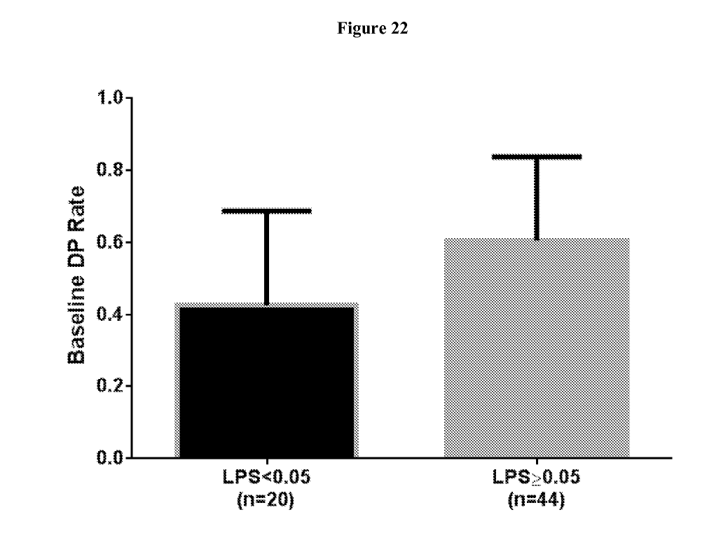

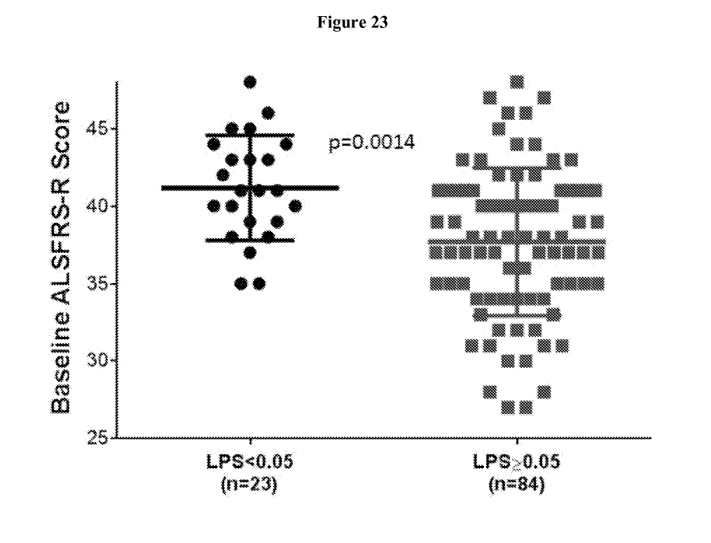

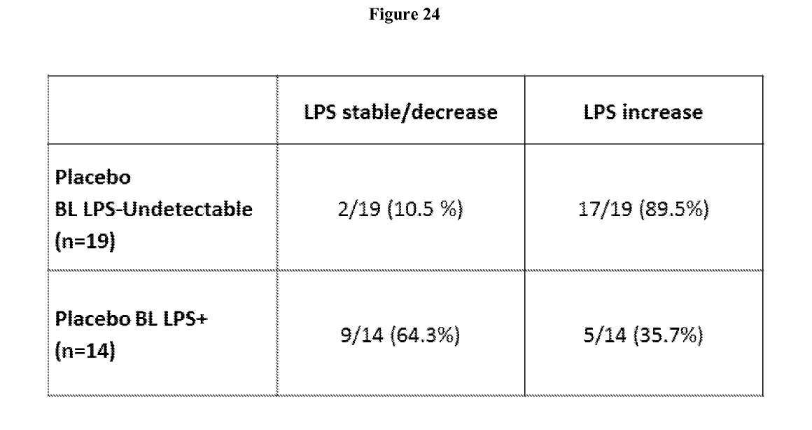

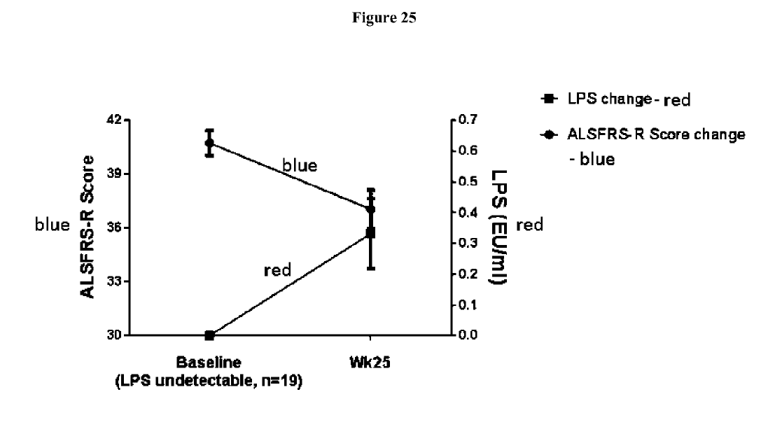

FIG. 22 shows that LPS baseline negative patients progress slower than patients with positive LPS baseline level. Plasma LPS as trial entry either + or −. Median progression rate calculated based on knowing trial entry date and date of symptom onset of all 64 patients (2 mg/kg and placebo groups). Progression rate was calculated based on symptom onset date. LPS baseline negative patients (41.2) also have higher baseline ALSFRS-R scores than LPS baseline positive patients (37.7) (see FIG. 23). In addition, LPS baseline negative patients without chlorite/NP001 treatment (placebo group) converted to LPS positive within 6 months as shown in FIG. 24 where plasma LPS level at 6 months is compared to baseline. These patients show dropping in ALSFRS-R functional scores (FIG. 25). In brief, 16/19 LPS negative patients converted to LPS positive; while 4/19 did not progress but developed LPS positive blood during trial.

### Example 6—Treatment of ALS

While positive responses to the chlorite treatment are found to be related to the initial concentration or baseline levels of biomarkers, different concentrations of biomarkers which are all above the cut-off value can cause different levels of responses to the treatment.

ALS patients with initial concentrations of biomarkers higher than the cut-off values are enrolled for trial. Different biomarkers including IL-18, IL-6, INF-g or CRP are studied. For each of the biomarkers, at least five baseline levels are selected and four groups are created with each pair of adjacent levels being used to define the range each group encompasses. Groups of patients are treated with 1-2 mg/kg/infusion sodium chlorite over the course of at least 6 months. ALSFRS-R and LPS levels are determined and recorded at different time points during the treatment, e.g., baseline (pre-dose Cycle 1, Week 1), 1-month, 2-month, 3-month, 4-month, 5-month and 6-month. A mean value of ALSFRS-R level for each group is calculated by averaging all its values taken at each time point. A mean value of baseline level in each group is determined by taking the average over all of biomarker baseline levels of the patients in that group. The mean values of ALSFRS-R levels are then plotted against the mean baseline values of biomarkers. For all of the biomarkers studied, a linear relationship with a positive slope between the mean values of ALSFRS-R levels and the mean baseline levels of biomarkers can be found out, which indicates that the degree of positive responses in patients to the treatment is proportional to the baseline levels of biomarkers.

A decrease in LPS level over the course of treatment also signals the positive responses to the treatment. More decreases in LPS level after the treatment indicates the better effect of the treatment. For each biomarker, LPS levels at baseline and week 25 are measured and recorded for all the groups. An absolute value of the difference between the levels at week 25 and baseline is calculated and averaged. A mean value of baseline level in each group is determined by taking the average over all of biomarker baseline levels of the patients in that group. The averaged absolute values of differences of biomarker levels after the treatment are then plotted against the mean baseline levels of biomarkers. For all the biomarkers, patients with lower baseline levels of biomarkers have less decreases in LPS levels and hence the poorer responses to the treatment.

### Example 7—Treatment of Alzheimer's Disease (AD)

AD patients with initial concentrations of biomarkers higher or lower than the cut-off values are enrolled for trial. Different biomarkers including IL-18, IL-6, INF-g or CRP are studied. For each of the biomarkers, at least five baseline levels are selected and four groups are created with each pair of adjacent levels being used to define the range each group encompasses.

Groups of patients are treated with 1-2 mg/kg/infusion sodium chlorite over the course of 6 months. Disease progression evaluation are determined and recorded at different time points during the treatment, e.g., baseline (pre-dose Cycle 1, Week 1), 1-month, 2-month, 3-month, 4-month, 5-month and 6-month.

It is expected that chlorite is capable of treating AD patients and AD patients with different biomarker level would respond to the chlorite treatment differently.

### Example 8—Treatment of PD

PD patients with initial concentrations of biomarkers higher than the cut-off values are enrolled for trial. Different biomarkers including IL-18, IL-6, INF-g or CRP are studied. For each of the biomarkers, at least five baseline levels are selected and four groups are created with each pair of adjacent levels being used to define the range each group encompasses. Groups of patients are treated with 1-2 mg/kg/infusion sodium chlorite over the course of 6 months. Parkinson's disease progression is evaluated and recorded at different time points during the treatment, e.g., baseline (pre-dose Cycle 1, Week 1), 1-month, 2-month, 3-month, 4-month, 5-month and 6-month.

It is expected that chlorite treatment is capable of treating Parkinson's patients and Parkinson's patients with different biomarker level would respond to the chlorite treatment differently.

### Example 9—NP001 Regulation of Macrophage Activation Markers in ALS

To assess the effects of NP001 administration on monocyte activation markers, a phase I, double blinded, placebo-controlled, single ascending dose safety and tolerability clinical study of NP001 in patients with ALS was conducted by Neuraltus Pharmaceuticals, Inc. (Palo Alto, Calif.), and the Western ALS Study Group (Clinicaltrials.org NCT01091142).

Thirty-two male and female with probable or definite ALS according to modified El Escorial criteria (Brooks et al., (2000) Research Group on Motor Neuron Diseases, 1(5): 293-299) were allocated in 5 groups: 1 placebo (8), or one of 4 (6 at each dose) ascending single iv doses (0.2, 0.8, 1.6 and 3.2 mg/kg NP001). Patients were included if age <75 years, stable riluzole dose for 30 days, and able to provide informed consent. Patients with tracheostomy, other active diseases besides ALS, or taking immunosuppressant therapy were excluded. Clinical features of the patients are listed in Table 4. Patients receiving either placebo or ascending doses of NP001 were monitored for the Primary endpoints of: safety and, changes in clinical status, and Secondary endpoints of: blood monocyte immune activation markers CD16 and HLA-DR responses to NP001 among blood monocytes at least 24 hours before dosing and at least 24 hours post-dosing. Changes from baseline in each monocyte marker were included in the statistical plan and those values were obtained by an independent flow cytometry laboratory at UCSF using validated procedures for the determinations. The statistical analysis was performed by an independent statistician for the CD16 values and by Neuraltus scientists for the HLA-DR values.

TABLE 4
Baseline ALS Characteristics (Safety Analysis Population)

| | | | | | | |
|---|---|---|---|---|---|---|
| | | NP001 | NP001 | NP001 | NP001 | All NP001 |
| | Placebo | 0.2 mg/kg | 0.8 mg/kg | 1.6 mg/kg | 3.2 mg/kg | Doses |
| Variable | (N = 8) | (N = 6) | (N = 6) | (N = 6) | (N = 6) | (N = 24) |

Duration of ALS Symptoms [months]

| | | | | | | |
|---|---|---|---|---|---|---|
| n = | 8 | 6 | 5 | 6 | 5 | 22 |
| Mean (Std) | 24.7 (15.7) | 22.4 (24.1) | 32.5 (21.3) | 21.3 (9.5) | 21.0 (10.5) | 24.1 (17.0) |
| Median | 19.1 | 14.5 | 28.9 | 18.2 | 24.8 | 19.4 |

Type of ALS [n (%)]

| | | | | | | |
|---|---|---|---|---|---|---|
| Familial | 0 (0.0) | 1 (16.7) | 1 (16.7) | 0 (0.0) | 0 (0.0) | 2 (8.3) |
| Sporadic | 8 (100.0) | 5 (83.3) | 5 (83.3) | 6 (100.0) | 6 (100.0) | 22 (91.7) |

Site of ALS Onset [n (%)]

| | | | | | | |
|---|---|---|---|---|---|---|
| Bulbar | 2 (25.0) | 3 (50.0) | 2 (33.3) | 0 (0.0) | 3 (50.0) | 8 (33.3) |
| Bulbar and Limb | 0 (0.0) | 0 (0.0) | 0 (0.0) | 2 (33.3) | 0 (0.0) | 2 (8.3) |
| Limb | 6 (75.0) | 3 (50.0) | 4 (66.7) | 4 (66.7) | 3 (50.0) | 14 (58.3) |

El Escorial ALS Criteria [n (%)]

| | | | | | | |
|---|---|---|---|---|---|---|
| Definite | 3 (37.5) | 2 (33.3) | 2 (33.3) | 3 (50.0) | 1 (16.7) | 8 (33.3) |
| Probable | 5 (62.5) | 4 (66.7) | 4 (66.7) | 3 (50.0) | 5 (83.3) | 16 (66.7) |

Riluzole use [n (%)]

| | | | | | | |
|---|---|---|---|---|---|---|
| No | 3 (37.5) | 2 (33.3) | 1 (16.7) | 2 (33.3) | 3 (50.0) | 8 (33.3) |
| Yes | 5 (62.5) | 4 (66.7) | 5 (83.3) | 4 (66.7) | 3 (50.0) | 16 (66.7) |

ALSFRS-R Score at Baseline

| | | | | | | |
|---|---|---|---|---|---|---|
| n = | 8 | 6 | 6 | 6 | 6 | 24 |
| Mean (Std) | 34.8 (5.2) | 34.5 (7.0) | 31.0 (7.8) | 30.2 (7.7) | 38.0 (5.2) | 33.4 (7.3) |
| Median | 34.0 | 37.0 | 30.5 | 31.0 | 39.0 | 34.5 |

Vital Capacity (L) at Baseline; n =

| | | | | | | |
|---|---|---|---|---|---|---|
| n = | 8 | 6 | 6 | 6 | 6 | 24 |
| Mean (Std) | 3.6 (1.6) | 2.1 (1.6) | 2.6 (1.5) | 3.3 (0.8) | 3.2 (0.8) | 2.8 (1.3) |
| Median | 3.9 | 2.4 | 2.8 | 3.1 | 3.1 | 3.1 |

### Informed Consent and Ethical Approval

The study was conducted at three clinical sites in the United States: California Pacific Medical Center, San Francisco, Calif.; University of Kansas Medical Center Research Institute, Kansas City, Kans.; University of Kentucky ALS Center, Lexington, Ky. Patients with ALS provided informed consent in accordance with guidelines established by California Pacific Medical Center and University of California San Francisco (UCSF) committees on human research, coordinated by the AIDS and Cancer Specimen Resource (ACSR). Similar approvals were obtained at the other two clinical sites. All research was conducted according to Declaration of Helsinki principles. Each participant was identified by number and not by name. Both patients and evaluators were blinded as to treatment assignment.

### Blood Monocyte Activation/Inflammation Assays

To explore the effects of single doses of NP001 on macrophage inflammatory activation markers potentially relevant to the pathogenesis and progression of ALS, the Revised ALS Functional Rating Scale (ALSFRS-R), scored 0-48, was used to evaluate overall patient functional status (Cedarbaum et al., (1999) Journal of the neurological sciences, 169(1-2): 13-21). Estimated disease progression rate was calculated as follows:

Mean monthly decline rate=(48−ALSFRS-R score at baseline)/Disease duration.

The monocyte activation markers measured were the levels of CD16 and HLA-DR expression on CD14+ monocytes from stained whole blood. CD16 and HLA-DR expression on CD14+ monocytes are measures of monocyte/macrophage inflammatory activation at the cellular level (Zhange et al., (2005) I Neuroimmunol. 159(1-2): 215-224; Belge et al., (2002) J Immunol 168(7): 3536-3542; Scherberich and Nockher, (1999) Clin Chem Lab Med. 37(3): 209-213; Ziegler-Heitbrock, (2007) Journal of leukocyte biology 81(3): 584-592; Merino et al., (2011) J Immunol 186(3): 1809-1815). Blood specimens for exploratory monocyte activation marker analysis were collected from patients before dosing and 24 hours post-dosing. Specimens were transported from the clinical site to a designated laboratory for same day sample preparation. Stained and fixed samples were then transported to the UCSF Core immunology laboratory (UCSF, San Francisco, Calif.) for flow cytometer measurement by LSR II flow cytometer (Becton Dickinson) using FACSDiva software (BD Biosciences, San Jose, Calif.).

Data was compensated and analyzed by FlowJo software (TreeStar Inc., Ashland, OR). The results from flow cytometry analysis were expressed as the geometric mean fluorescence intensity (Geo MFI) of monocyte activation markers. A typical gating strategy used to identify HLA-DR and CD16 expression on CD14+ monocytes by flow cytometry included: CD3 and CD16 were used to exclude the CD3+ lymphocytes and CD16+ granulocytes that contaminate in the mononuclear cell gate. CD14+HLA-DR+ cells were then gated from a HLA-DR vs. CD14 dot plot which excludes remaining lymphocytes including B cells and NK cells. Total monocytes were then gated on a CD14 vs. side scatter plot (CD14+). From the CD14+ gate the geometric mean fluorescence intensity (MFI) of HLA-DR were measured. The proportion of CD16+ and CD16 bright cells were also gated from the CD14+ cells on a CD14 vs. CD16 dot plot. CD16 bright gate (in general 10× brighter than standard CD16 intensity) captures all the dim CD14+ CD16+ bright cells.

### Safety and Clinical Status Variables

After NP001 treatment, patients were monitored for a variety of safety and clinical status variables during and for 8 hours after infusions and at 1, 4 and 7 days after dosing. This included physical examinations, including inspection of the infusion site for reactions, and clinical lab tests involving blood counts, a multi-channel chemistry panel, urinalysis, electrocardiograms and vital capacity. Safety data from the full cohort of 8 patients from each dose level was reviewed by the safety monitoring committee before escalating to the next higher dose. Flow cytometer assessment of NP001 treatment in blood monocyte was performed before dosing and 24 hours post-dosing.

### Statistical Analysis

Statistical analysis was performed by GraphPad Prism 6.0 program (GraphPad Software, San Diego, Calif., USA). Flow cytometer results were expressed as the mean±SED unless otherwise stated. Statistical significance was assessed using One-way ANOVA, and linear regression, as indicated in the table and figure legends. For all analysis, a value of p<0.05 was considered statistically significant.

### Safety Results

This Phase I safety and tolerability study of NP001 in subjects with ALS was completed by the Western ALS Study Group and Neuraltus Pharmaceuticals, Inc. in 2010. In this trial, 32 patients (clinical features in Table 4) were enrolled and four cohorts of patients received a single dose of NP001 (0.2, 0.8, 1.6 or 3.2 mg/kg NP001 chlorite, N=6 per cohort, total 24 NP001 patients) or placebo (saline, N=2 per cohort, total 8 placebo patients) as a 30-minute infusion on Day 1. All doses of NP001 were generally safe and well-tolerated and there were no treatment-related serious adverse events (Table 5) or clinically relevant changes in safety associated laboratory parameters. In addition, blood monocyte activation markers, CD16 and HLA-DR, were quantitated at baseline and 24 hours after a single dose of the drug or placebo infusion.

TABLE 5
Summary of Treatment-Emergent Adverse Events That Occurred in ≧2 Subjects in the All
NP001 Doses or Placebo Groups (Safety Analysis Population)

| | | | | | | All |
|---|---|---|---|---|---|---|
| | | NP001 | NP001 | NP001 | NP001 | NP001 |
| System Organ Class | Placebo | 0.2 mg/kg | 0.8 mg/kg | 1.6 mg/kg | 3.2 mg/kg | Doses |
| Preferred Term | (N = 8) | (N = 6) | (N = 6) | (N = 6) | (N = 6) | (N = 24) |
| Subjects with a TEAE that Occurred | 1 | 2 | 1 | 2 | 2 | 7 |
| in ≧2 Subjects in the All NP001 | | | | | | |
| Doses or Placebo Groups | | | | | | |
| Fall | 0 | 1 | 1 | 0 | 2 | 4 |
| Contusion | 0 | 1 | 0 | 1 | 0 | 2 |
| Facial pain | 0 | 1 | 0 | 0 | 1 | 2 |
| Fatigue | 1 | 1 | 0 | 1 | 0 | 2 |

Baseline monocyte/macrophage activation-related inflammatory cell surface markers are increased in ALS patients in relation to rate of ALS disease progression.

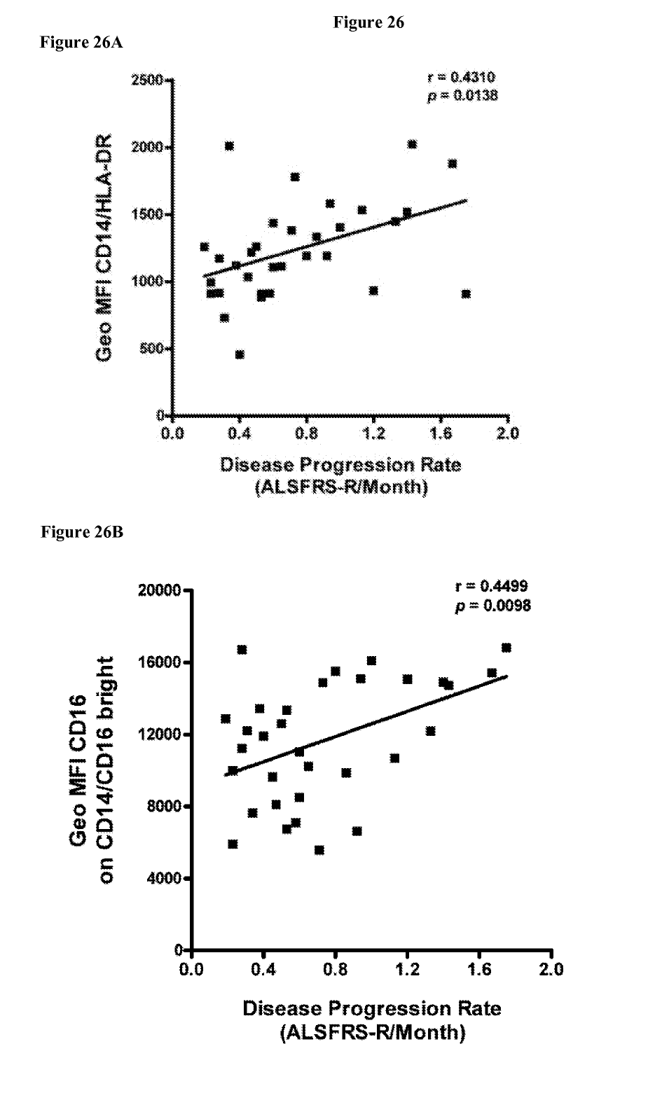

In a previous report, the degree of systemic monocyte/macrophage activation (monocyte overexpression of both HLA-DR and CD16), was found to be associated with the rate of ALS disease progression (Zhang et al., (2005) J Neuroimmunol 159(1-2): 215-224); the higher the level of activation the more rapid the ALS disease progression. ALS patient blood monocytes obtained at baseline in the NP001 phase I study showed evidence for monocyte activation as defined by CD14 cell co-expression of HLA-DR, levels of which were related to the estimated rate of ALS disease progression (ALSFRS-R Score loss per month based on evaluation of patient symptom duration) (r=0.4310, p=0.0138; n=32) (FIG. 26A). A positive correlation was also observed between the ALS disease progression rate and levels of CD16 on CD16 bright monocytes, the most activated subset of proinflammatory monocytes that act as differentiated monocytes or tissue macrophages (Belge et al., (2002) J Immunol 168(7): 3536-3542; Ziegler-Heitbrock, (2007) Journal of leukocyte biology 81(3): 584-592; Sadeghi et al., (1999) Experimental gerontology 34(8): 959-970; Takeyama et al., (2007) Annals of hematology 86(11): 787-792; Thieblemont et al., (1995) European journal of immunology 25(12): 3418-3424) (404244-46) (r=0.4499, p=0.0098, n=32) (FIG. 26B). Moreover, a multiple regression analysis revealed that the two monocyte activation markers were independent of each other in relationship to ALS disease progression rate, and when combined showed an enhanced association with rate of ALS disease progression (Multiple R=0.5734, p=0.0031). No relationship was found between baseline ALSFRS-R score and levels of either monocyte HLA-DR or monocyte CD16 bright subset co-expression. NP001 decreases level of monocyte HLA-DR in patients with elevated HLA-DR values at baseline

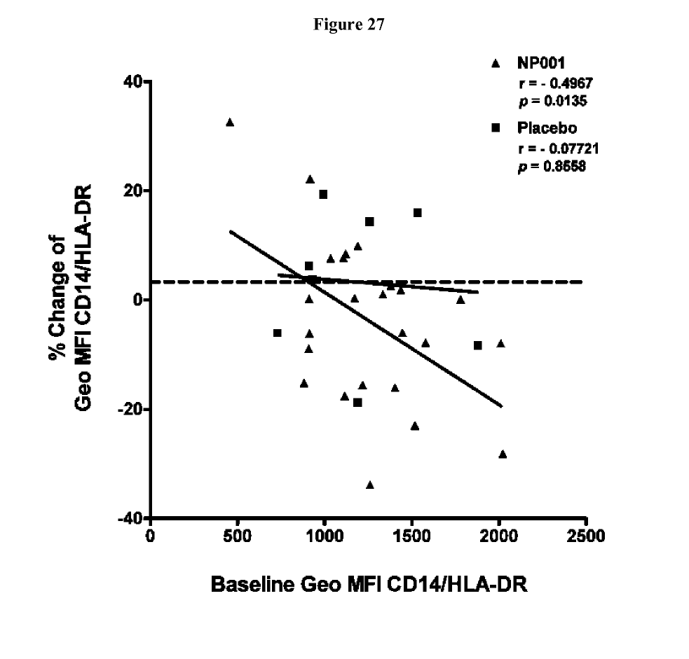

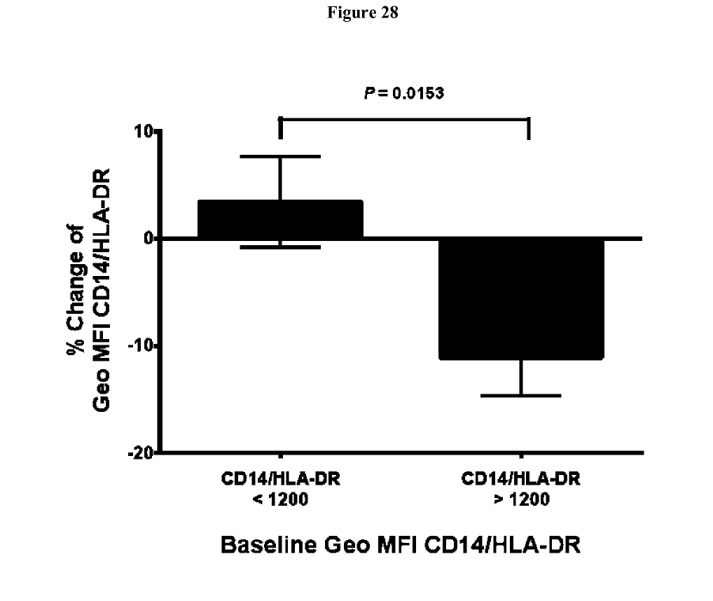

Following NP001 treatment, changes in monocyte levels of HLA-DR did not demonstrate a dose-dependent effect; however HLA-DR expression was down regulated at all doses of NP001 in patients with the high baseline levels of monocyte HLA-DR. FIG. 27 shows the scatter plot of change in NP001-induced monocyte HLA-DR expression levels as a function of monocyte HLA-DR baseline levels for the 32 subjects dosed in the NP001 phase I study. The x-axis represents the baseline values of the geometric mean fluorescence intensity of monocyte HLA-DR expression (Geo MFI CD14/HLA-DR). The y-axis represents the percent change from baseline in total monocyte HLA-DR expression. The red line represents the mean percentage change of HLA-DR expression on monocytes from 8 placebo patients; the black boxes and line represent the actual individual change from placebo group (r=−0.07721, p=0.8558; N=8). The blue triangles and line represent the change in monocyte HLA-DR expression after NP001 independent of dose (r=−0.4967, p=0.0135; N=24). The placebo group showed relatively stable monocyte HLA-DR after treatment (r=−0.07721, p=0.8558; N=8). The changes of HLA-DR expression on monocytes in the NP001 treatment response were linearly related to the degree of baseline monocyte HLA-DR expression 24 hours after treatment (r=−0.4967, p=0.0135; N=24). The greater the starting monocyte HLA-DR levels at baseline, the greater the HLA-DR response to NP001. A representation of the data based on starting monocyte HLA-DR levels at baseline is shown in FIG. 28. Patients treated with NP001 were divided into two groups based on the median value of baseline monocyte HLA-DR (Geo MFI CD14/HLA-DR=1200) from the entire group of all 32 patients enrolled in the phase I clinical study. Baseline Geo MFI CD14/HLA-DR were clustered into two groups as shown on the x-axis (Geo MFI CD14/HLA-DR>1200, N=12; Geo MFI CD14/HLA-DR<1200, N=12). The y-axis represents the percent change in monocyte geometric mean levels of HLA-DR at 24 hours as compared to baseline. Positive values show an increase in HLA-DR expression and negative values show a relative decrease in HLA-DR expression. In the group of 12 patients with elevated baseline monocyte HLA-DR the average % change from baseline 24 hours after NP001 was more than 10%, whereas those patients with lower range monocyte HLA-DR showed no change from baseline (p=0.0153).

NP001 Associated Change in Monocyte HLA-DR Expression is Associated with the Estimated Rate of ALS Disease Progression

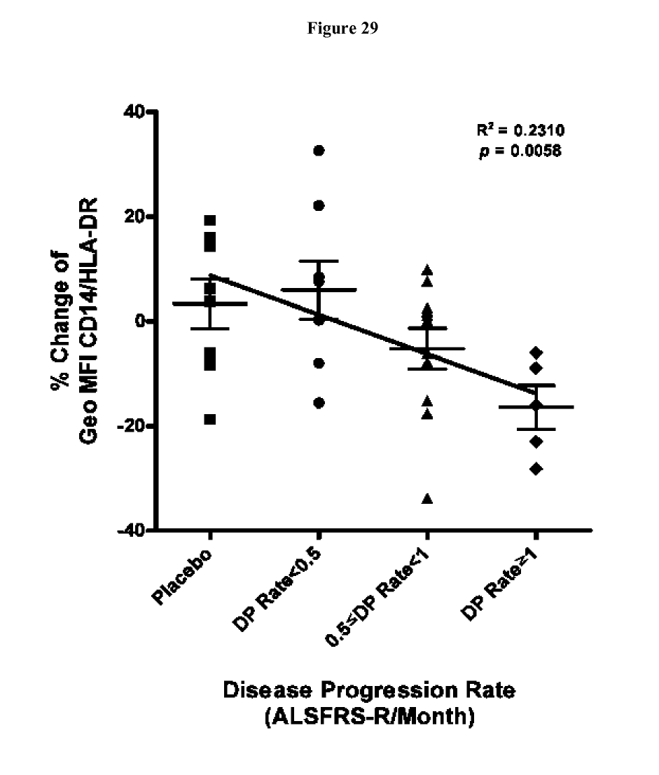

A post-hoc analysis to evaluate the effect of ALS estimated disease progression rate on these results was conducted. FIG. 29 shows the results of monocyte HLA-DR expression change after NP001 treatment (pooled data) as a function of each patient's historical rate of ALS disease progression since onset of symptoms (based on review of clinical charts at the participating institutions). Patients in the phase I trial were clustered into subgroups based on their historic rate of ALS disease progression, assessed by average monthly change on ALSFRS-R (DP Rate=disease progression rate) and compared to placebo group (N=8). DP rates were clustered into three groups as showed on the x-axis (DP Rate<0.5, N=8; DP Rate between 0.5 and 1, N=11; DP Rate≧1, N=5). The y-axis represents the percent change in monocyte geometric mean levels of HLA-DR at 24 hours as compared to baseline. Positive values show an increase in HLA-DR expression and negative values show a relative decrease in HLA-DR expression. R²=0.2310, p=0.0058, One-way ANOVA followed by posttest for linear trend. The average ALS patient declined at a rate of approximately 1 unit/month using the ALSFRS-R scoring scale. Patients who were slow progressors (defined as estimated rates of progression <0.5 unit per month) showed no change in HLA-DR regardless of whether the patient received NP001 or placebo. In contrast, patients with estimated rates of progression ≧1 unit per month showed the greatest change in HLA-DR expression following NP001 dosing (R²=0.2310, p=0.0058 for the linear trend comparison). NP001 induces a dose-dependent decreased level of CD16 expression the bright CD16 subset of CD14 monocytes in vivo.

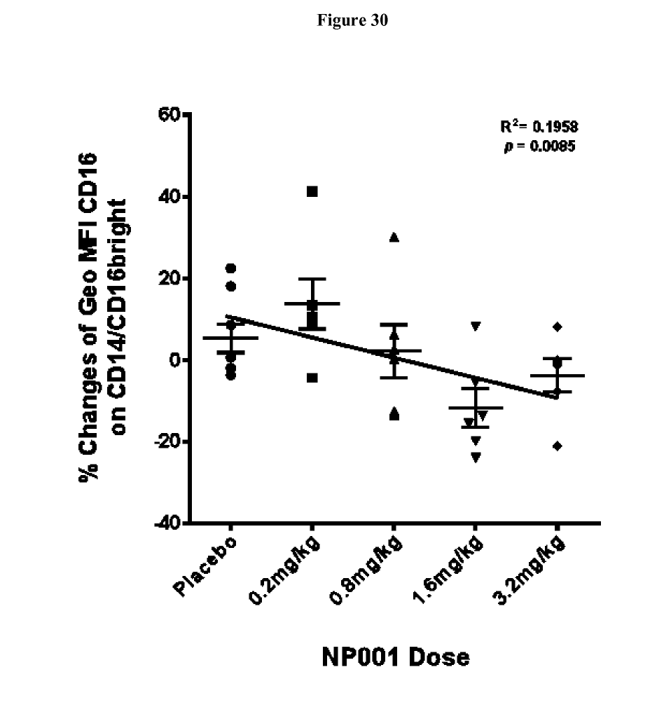

Dose-dependent changes in NP001 treated patients as compared to placebo were observed in the level of CD16 expression on the CD16 bright subset of monocytes. The degree of monocyte CD16 modulation was not correlated with baseline CD16 expression or the estimated rate of decline as assessed by the change in ALSFRS-R since disease onset. FIG. 30 shows the dose-dependent relationship trend between change in monocyte CD16 expression from baseline and the dose of NP001 administered (R²=0.1958, p=0.0085 for the linear trend comparison; placebo, N=8; NP001, N=6 for each dose). ALS patients treated with a single dose of NP001 or placebo had baseline values of monocyte CD16 expression compared with the same measurement obtained 24 hours after dosing. The percent change in CD16 level expressed on a CD16 bright subset of monocytes 24 hours after dosing are plotted on the y-axis. Placebo (N=8) and dose levels (N=6 for each dose) are plotted on the x-axis. R²=0.1958, p=0.0085, One-way ANOVA followed by posttest for linear trend. Note that there was no significant change in the level of monocyte CD16 expression in the placebo group.

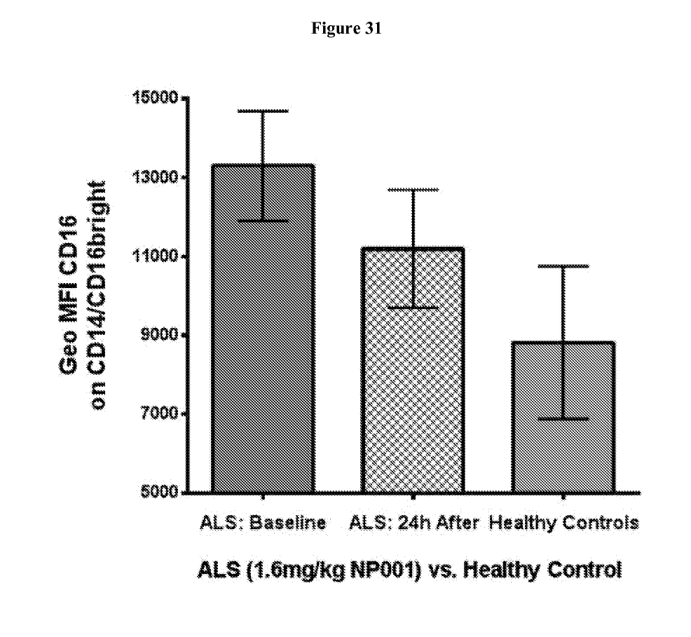

FIG. 31 shows the absolute level of CD16 in the monocyte CD16 bright subset from patients who received the 1.6 mg/kg dose of NP001 as defined by quantitative flow cytometry. The left and middle bars represent mean levels of CD16 expression on ALS patient CD16 bright monocytes at baseline (left) and 24 hours after NP001 infusion (middle) (N=6). The bar on the right represents mean level of CD16 expression typically seen in healthy controls (N=7). Twenty four hours after one dose of NP001, the difference between the ALS and normal control level of monocyte CD16 expression was reduced by approximately 50% toward the normal value compared with baseline pretreatment levels in the ALS patients.

The phase 1 study of NP001 in patients with ALS, is associated with two definable effects on monocyte/macrophage activation in patients with elevated inflammatory markers at baseline: 1) a systemic anti-inflammatory effect and 2) a marked decrease in the CD16 level in a subpopulation of monocytes that are capable of migrating from blood into tissues. There were no safety or tolerability issues identified. Without being bound by any theory, NP001 treatment may reduce both systemic inflammation and blood monocyte migration into the spinal cord, key processes thought to be critical to the progression of ALS, with the potential to slow the progression of the disease.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of treating a subject suffering from a macrophage-related disease, said method comprising:
a) selecting a subject suffering from a macrophage-related disease if said subject has an elevated plasma level of one or more inflammatory factors chosen from the group consisting of LPS, IL-6, IL-8, IL-18, IFN-g, and CRP; and
b) administering to the subject a therapeutically effective amount of a pharmaceutical composition comprising chlorite.

2. The method of claim 1, wherein the one or more inflammatory factors is IL-18.

3. The method of claim 1 or 2, wherein the plasma level of IL-18 prior to said administering is at least about 60 pg/ml.

4. The method of claim 1 or 2, wherein the plasma level of IL-18 in said subject decreases after said administering.

5. The method of claim 1 or 2, wherein the subject further has an elevated plasma level of one or more inflammatory factors selected from the group consisting of: LPS, IL-6, IL-8, IFN-g, and CRP.

6. The method of claim 1, wherein the one or more inflammatory factors is LPS.

7. The method of claim 1 or 6, wherein said subject further has an elevated plasma level of one or more inflammatory factors selected from the group consisting of IL-18, IL-6, IL-8, IFN-g, and CRP.

8. The method of claim 1 or 6, wherein the plasma level of LPS prior to said administering is at least about 0.05, 0.1, 0.15, or 0.2 EU/ml.

9. The method of claim 1, 6 or 8, wherein the serum level of LPS prior to said administering is at least about 0.05 EU/ml.

10. The method of claim 1 or 6, wherein the plasma level of LPS in said subject is higher than the normal level prior to said administering.

11. The method of claim 1 or 6, wherein the plasma level of LPS in said subject decreases after said administering.

12. The method of claim 1 or 6, wherein the plasma level of LPS in said subject decreases to an undetectable level after said administering.

13. The method of claim 1, wherein the subject has elevated plasma levels of IL-6 and IFN-g.

14. The method of claim 1 or 13, wherein the plasma level of IL-6 is at least about 6 pg/ml.

15. The method of claim 1 or 13, wherein the serum level of IFN-g is at least about 20 pg/ml.

16. The method of claim 1, wherein the serum level of CRP is at least about 1000 ng/ml.

17. The method of claim 1 or 16, wherein said subject has an elevated serum level of at least two inflammatory factors chosen from the group consisting of LPS, IL-6, IL-8, IL-18, IFN-g and CRP.

18. The method of any of the preceding claims, wherein the macrophage-related disease is selected from amyotrophic lateral sclerosis (ALS), Alzheimer's disease (AD) and Parkinson's disease (PD), and HIV-associated neurocognitive disorder (HAND.

19. The method of claim 18, wherein the macrophage-related disease is amyotrophic lateral sclerosis (ALS).

20. The method of claim 1, wherein said subject was diagnosed as having the macrophage-related disease less than 3 years prior to said administering.

21. The method of claim 1, wherein said subject does not show disease progression for at least 6 months after said administering.

22. The method of any of the preceding claims, wherein said chlorite is administered in an amount of at least 0.2, 0.8, 1.0. 1.2, 1.4, 1.6, 1.8, 2.0, or 3.2 mg/kg body weight.

23. The method of claim 22, wherein said chlorite is administered in an amount of at least 1 mg/kg or at least 2 mg/kg body weight.

24. The method of any of the preceding claims, wherein said composition is administered intravenously.

25. The method of any of the preceding claims, wherein said composition is administered at least twice, three times or five times per month.

26. The method of any of the preceding claims, wherein said composition is administered for at least 2, 3, 4, 5 or 6 months.

27. The method of any of the preceding claims, wherein said chlorite is greater than 95%, 99% or 99.5% pure.

28. The method of any of the preceding claims, wherein said composition further comprises a pH adjusting agent.

29. The method of claim 28, wherein said composition is a liquid that exhibits 25% less pH drift compared to an identical composition without said pH adjusting agent.

30. The method of claim 28 or 29, wherein said pH adjusting agent is a phosphate buffer.

31. The method of any of the preceding claims, wherein said chlorite is sodium chlorite.

32. The method of any one of claims 1-30, wherein said chlorite is in a form of WF10.

33. The method of claim 1, wherein said chlorite is administered for at least 2, 3, 4, 5 or 6 months.

34. A method of monitoring the inflammation progress of a macrophage-related disease in a subject comprising:
a) administering to the subject a pharmaceutical composition comprising chlorite;
b) measuring the plasma level of at least one monocyte activation marker selected from the group consisting of HLA-DR and CD16;
c) comparing the measured plasma level of said monocyte activation marker to a plasma level of said monocyte activation marker in the subject prior to said administering step; and
d) continuing to administer the pharmaceutical composition to the patient if the plasma level of said monocyte activation marker has changed as compared to the plasma level of said monocyte activation marker prior to said administering.

35. The method of claim 34, wherein said plasma level of at least one monocyte activation marker is measured 24 hours prior to said administering.

36. The method of claim 34, wherein said plasma level of at least one monocyte activation marker is measured 24 hours after said administering.

37. The method of any one of claims 34-36, wherein said monocyte activation marker is HLA-DR.

38. The method of any one of claims 34-37, wherein the plasma level of HLA-DR is higher than normal level prior to said administering.

39. The method of any one of claims 34-38, wherein the plasma level of HLA-DR decreases after said administering.

40. The method of any one of claims 34-39, further comprising measuring the plasma level of CD14.

41. The method of claim 40, wherein the plasma level of CD14 is higher than normal level prior to said administering.

42. The method of any one of claims 34-41, wherein the plasma level of CD14 decreases after said administering.

43. The method of claim 34, wherein said monocyte activation marker is CD16.

44. The method of claim 34, wherein said monocyte activation marker is higher than normal level prior to said administering.

45. The method of any one of claims 34-44, wherein the plasma level of CD16 decreases after said administering.

46. The method of any one of claims 34-45, wherein elevation of the plasma level of said monocyte activation marker is correlated with the rate of progression of said macrophage-related disease.

47. The method of claim 34, wherein the elevated plasma level of HLA-DR and CD16 increase the rate of progression of said macrophage-related disease.

48. The method of any one of claims 34-47, wherein said administering decreases the progression of said macrophage-related disease.

49. The method of any one of claims 34-48, wherein said administering decreases the progression of said macrophage-related disease by at least 1.0 unit/month using the ALSFRS-R scoring scale.

50. The method of any one of claims 34-49, wherein said subject suffering from a macrophage-related disease has progression rate of at least 0.5 unit/month using the ALSFRS-R scoring scale.

51. The method of any one of claims 34-50, wherein said subject suffering from a macrophage-related disease has progression rate of at least 1.0 unit/month using the ALSFRS-R scoring scale.

52. The method of any one of claims 34-51, wherein the macrophage-related disease is selected from amyotrophic lateral sclerosis (ALS), Alzheimer's disease (AD), Parkinson's disease (PD), and HIV-associated neurocognitive disorder (HAND).

53. The method of claim 52, wherein the macrophage-related disease is amyotrophic lateral sclerosis (ALS).
